(19) United States
(12) Patent Application Publication
(10) Pub. No.: US 2023/0129792 A1
(43) Pub. Date: Apr. 27, 2023

(54) TISSUE CUTTING ELEMENTS AND RETENTION FEATURES FOR IMPLANTS, AND ASSOCIATED SYSTEMS AND METHODS

(71) Applicant: W. L. Gore & Associates, Inc., Newark, DE (US)

(72) Inventors: Cody L. Hartman, Flagstaff, AZ (US); Ryan S. Titone, Flagstaff, AZ (US)

(21) Appl. No.: 17/909,591

(22) PCT Filed: Mar. 2, 2021
(86) PCT No.: PCT/US2021/020446

§ 371 Date: Sep. 6, 2022

Related U.S. Application Data
(60) Provisional application No. 62/984,564, filed on Mar. 3, 2020.

(51) Int. Cl. A61F 2/24; A61B 18/14

(52) U.S. Cl. CPC A61F 2/2418; A61B 18/14; A61B 2018/00369

(57) ABSTRACT

Prosthetic valves and associated methods and systems are disclosed, including a prosthetic valve with a support structure and a leaflet construct coupled to the support structure, and a tissue cutting element coupled to a portion of the prosthetic valve.

### CROSS-REFERENCE TO RELATED APPLICATION

This application is a national phase application of PCT Application No. PCT/US2021/020446, internationally filed on Mar. 2, 2021, which claims the benefit of Provisional Application No. 62/984,564, filed Mar. 3, 2020, which are incorporated herein by reference in their entireties for all purposes.

### FIELD

The present disclosure relates generally to implants, such as a prosthetic valve, including one or more tissue cutting elements and associated devices, systems and methods.

### BACKGROUND

Prosthetic valves have been developed to replace native valves or otherwise augment physiology. Prosthetic valves may employ flexible leaflets fabricated from biological tissue or synthetic materials. Many conventional prosthetic valve designs require delivery to a target region within a patient's anatomy via open-heart surgical techniques, though alternative approaches using transcatheter techniques have been developed and can offer advantages. Among other examples, a transcatheter prosthetic valve that is delivered endovascularly can help minimize patient trauma as compared to surgical procedures.

However, transcatheter techniques are not without their own challenges, including properly accessing treatment regions within the anatomy, properly positioning the prosthetic valve for deployment, and depending on the particular anatomy being repaired or augmented, unwanted impact from surrounding anatomy on prosthetic valve performance or unwanted impact from the prosthetic valve on the performance of the surrounding tissue, each of which can negatively impact patient health.

### SUMMARY

Various examples address prosthetic valves including a support structure and a leaflet construct coupled to the support structure, as well as a tissue cutting element coupled to the support structure, also referred to as an on-board cutting element. The tissue cutting element may be used in a variety of contexts, but one particular use is for cutting heart valve tissue, such as one or more native leaflets of a heart valve. For example, the tissue cutting element may be configured and utilized for cutting an anterior leaflet of a mitral valve to avoid left ventricular output tract (LVOT) obstruction.

According to a first example (“Example 1), a prosthetic valve includes a support structure, one or more leaflets coupled to the support structure, and a tissue cutting element coupled to the support structure.

According to another example (“Example 2”), further to Example 1, the tissue cutting element is integral to the support structure.

According to another example (“Example 3”), further to any preceding Example, the tissue cutting element is configured to transitioned from an inactive state where the tissue cutting element is inoperable to cut tissue to an active state in which the tissue cutting element is operable to cut tissue.

According to another example (“Example 4”), further to Example 3, the tissue cutting element is configured to be held proximate the support structure in the inactive state when the tissue cutting element is inoperable to cut tissue and to project away from the support structure in the active state when the tissue cutting element is operable to cut tissue.

According to another example (“Example 5”), further to any preceding Example, the tissue cutting element has one or more cutting edges that are relatively sharp.

According to another example (“Example 6”), further to any preceding Example, the tissue cutting element has one or more cutting edges that are relatively dull such that the one or more cutting edges are operable to cut through tissue by eroding through the tissue over an extended period of time.

According to another example (“Example 7”), further to any preceding Example, the tissue cutting element extends a length from a base coupled to a portion of the prosthetic valve to a free end, and further wherein the tissue cutting element includes one or more cutting edges that extend for less than the length of the tissue cutting element.

According to another example (“Example 8”), further to any preceding Example, the tissue cutting element is configured as an electrosurgical cutting element.

According to another example (“Example 9”), further to any preceding Example, the tissue cutting element has a first cutting edge facing a first direction and a second cutting edge facing a second direction different than the first direction.

According to another example (“Example 10”), further to any preceding Example, the support structure includes a leaflet frame subcomponent to which the one or more leaflets and the tissue cutting element are coupled, and, optionally, wherein the support structure further includes an anchor frame subcomponent into which the leaflet frame subcomponent is configured to be at least partially, telescopically received, and, optionally wherein an inner diameter of the anchor frame subcomponent is greater than an outer diameter of the leaflet frame subcomponent.

According to another example (“Example 11”), further to any preceding Example, the support structure includes an interstage coupling the leaflet frame subcomponent and the anchor frame subcomponent.

According to another example (“Example 12”), further to any preceding Example, the support structure is configured to retain the tissue cutting element in an inactive state prior to deployment of the support structure.

According to another example (“Example 13”), further to any preceding Example, the tissue cutting element is configured to grasp and retain tissue in addition to cutting tissue.

According to another example (“Example 14”), further to any preceding Example, the tissue cutting element is formed of material that is at least one of bioresorbable and biocorrodible.

According to another example (“Example 15”), further to any preceding Example, the tissue cutting element is elastically deformable so as to be self-expanding under spring bias forces to project away from the support structure.

According to another example (“Example 16”), further to any preceding Example, the interstage comprises a polymer membrane.

According to another example (“Example 17”), further to any preceding Example, the prosthetic valve is configured to replace a native mitral valve and the tissue to be cut is that of an anterior leaflet of the native mitral valve.

According to another example (“Example 18”), further to any preceding Example, the one or more leaflets are flexible leaflets and, optionally, formed of synthetic material.

According to another example (“Example 19”), further to any preceding Example, the tissue cutting element is a single-edged cutting element.

According to another example (“Example 20”), further to any preceding Example, the tissue cutting element is a double-edged cutting element.

According to another example (“Example 21”), further to any preceding Example, the tissue cutting element has a cutting edge with a radius of curvature of between 0.1 μm and 5 μm.

According to another example (“Example 22”), further to any one of Examples 1 to 20, the tissue cutting element has a cutting edge with a radius of curvature of between 10 μm and 100 μm.

According to another example (“Example 23”), a method of delivering the prosthetic valve of any one of Examples 1 to 22 to a treatment site, the method comprising: positioning the prosthetic valve at the treatment site; and engaging the cutting element with native leaflet tissue at the treatment site to cut the native leaflet tissue, and, optionally, to grasp the native leaflet tissue.

According to another example (“Example 24”), further to Example 23, the method further comprises nesting a portion of the support structure, optionally a leaflet frame subcomponent, that is coupled to the cutting element with another portion of the prosthetic valve, optionally an anchor frame subcomponent.

According to another example (“Example 25”), further to the method of Examples 23 or 24, the native leaflet tissue is not cut at a time the cutting element is first engaged with the native leaflet tissue.

According to another example (“Example 26”), further to the method of Examples 23 or 24, the native leaflet tissue is cut at a time the cutting element is first engaged with the native leaflet tissue.

According to another example (“Example 27”), a method for treating a human patient with a diagnosed condition or disease associated with valve insufficiency or valve failure of a native valve includes implanting a prosthetic valve according to any of Examples 1 to 22 at or adjacent to a location associated with the native valve.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a side view of a prosthetic valve, according to some embodiments;

FIG. 1B is a side view of a prosthetic valve, according to some embodiments;

FIG. 1C is a perspective view of the prosthetic valve of FIG. 1A, according to some embodiments;

FIG. 1D is an axial view of the prosthetic valve of FIG. 1A, according to some embodiments;

FIG. 2A is a side view of a leaflet frame subcomponent, according to some embodiments;

FIG. 2B is an axial view of a leaflet frame subcomponent, according to some embodiments;

FIG. 3A is a side view of an anchor frame subcomponent, according to some embodiments;

FIG. 3B is an axial view of the anchor frame subcomponent, according to some embodiments;

FIGS. 3C is a side view of a prosthetic valve, according to some embodiments;

FIGS. 3D to 3G illustrate various tissue cutting element configurations, according to some embodiments;

FIG. 4 is an illustration of a delivery device, according to some embodiments;

FIGS. 5A to 5F are cross-sectional views of a heart illustrating an exemplary delivery procedure, according to some embodiments;

FIGS. 6A and 6B are cross-sectional views of a prosthetic valve deployed in an anatomy, according to some embodiments;

FIGS. 7A and 7B are side views of a portion of a prosthetic valve, according to some embodiments;

FIGS. 8A and 8B are perspective views of a portion of a prosthetic valve, according to some embodiments;

FIG. 9A is a front view of a prosthetic valve with flow enabling features in an open configuration, according to some embodiments;

FIG. 9B is a front view of the prosthetic valve of FIG. 9A with the flow enabling features in a closed configuration, according to some embodiments;

FIG. 9C is a front view of a prosthetic valve with flow enabling features, according to some embodiments;

FIG. 10A is a side view of a prosthetic valve in a delivery configuration, according to some embodiments;

FIG. 10B is a perspective view of the prosthetic valve of FIG. 10A in a deployed configuration, according to some embodiments;

FIG. 10C is a side view of a prosthetic valve in a delivery configuration, according to some embodiments;

FIG. 10D is a perspective view of the prosthetic valve of FIG. 10C in a deployed configuration, according to some embodiments;

FIG. 10E is a side view of a prosthetic valve in a delivery configuration, according to some embodiments;

FIG. 10F is a side view of a prosthetic valve in a delivery configuration, according to some embodiments;

FIGS. 11 and 12 are flow diagrams of methods of operating the prosthetic valve according to some embodiments.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about,” “approximate,” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example.

The term “membrane” as used herein refers to a sheet of material comprising a single composition, such as, but not limited to, expanded fluoropolymer.

The term “composite material” as used herein refers to a material including two or more material components with one or more different material properties from the other. In some examples, a composite material includes at least a first material component in the form of a membrane and a second material component in the form of a polymer that is combined with the membrane (e.g., by coating and/or imbibing processes). The term “laminate” as used herein refers to multiple layers of membrane, composite material, or other materials, such as, but not limited to a polymer, such as, but not limited to an elastomer, elastomeric or non-elastomeric material, and combinations thereof.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

The term “film” as used herein generically refers to one or more of the membrane, composite material, or laminate. The term “film” also includes fabric and other suitable materials.

The term “biocompatible material” as used herein generically refers to a film or a biological material, such as, but not limited to, bovine pericardium.

As used herein, “couple” means to join, connect, attach, adhere, affix, or bond, whether directly or indirectly, and whether permanently or temporarily.

As used herein, “native leaflet” is used to describe the leaflet of a native heart valve.

The term “leaflet” or “leaflet construct” which comprises a plurality of leaflets as used herein in the context of prosthetic valves is a component of a one-way valve wherein the leaflet is operable to move between an open and closed position under the influence of a pressure differential. In an open position, the leaflet allows fluid (e.g., blood) to flow through the valve. In a closed position, the leaflet substantially blocks retrograde flow through the valve by occluding the valve orifice. In embodiments comprising multiple leaflets, each leaflet cooperates with at least one neighboring leaflet or secondary structure to block the retrograde flow of blood. The pressure differential in the blood is caused, for example, by the contraction of a ventricle or atrium of the heart, such pressure differential typically resulting from a fluid pressure building up on one side of the leaflets when closed, for example, by the contraction of a ventricle or atrium of the heart. As the pressure on an inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve rises above the blood pressure on the inflow side of the valve, the leaflet returns to the closed position generally preventing retrograde flow of blood through the valve.

It will be appreciated that leaflets, where not required by the specific design or mode of function of the disclosed embodiment, may be rigid such as in mechanical valves or may be flexible as in bioprosthetic and synthetic valves. It will further be appreciated that, although embodiments provided herein include a frame that supports the leaflets, the leaflets may not necessarily be supported by a frame. In other embodiments, the leaflets may be constructed as in the tissue valve art that are formed into the desired shape without a frame.

The term “cutting” as used in the context of the present disclosure includes slicing, splitting, puncturing, tearing, and/or physically separating material, such as body tissue. For example, when a native leaflet as previously defined is cut, the resulting native leaflet may be partially split, torn, or separated to form a gap between two or more subsections thereof. The definition of “cut” may further include a result of one or more incision, laceration, bisection, separation, etc. Furthermore, the native leaflet does not necessarily need to be completely separated into two or more components that can move freely relative to each other; for example, the native leaflet may be at least partially separated, where the definition of “partially” may include 10%, 20%, 30%, 40%, 50%, 60%, 70%, 80%, 90%, or any range therebetween of a diameter or an edge-to-edge distance of the native leaflet to be cut.

As used herein, “native valve orifice” refers to a location into which the prosthetic valve may be placed. A native valve orifice includes a tissue orifice which includes anatomical structures into which a prosthetic valve can be placed. Such anatomical structures include, but are not limited to, a location wherein a cardiac valve may or may not have been surgically removed. Other anatomical structures that can receive a prosthetic valve include, but are not limited to, veins, arteries, ducts and shunts. A native valve orifice may also refer to a location in a synthetic or biological conduit that may receive a prosthetic valve.

### Description of Various Embodiments

The present disclosure relates generally to implants, such as a prosthetic valve, including one or more tissue cutting elements and associated devices, systems and methods. Some examples relate to prosthetic valves used for cardiac valve replacement or other applications associated with heart valves or other valve orifices, and related systems, methods, and apparatuses. In some examples, a prosthetic valve includes a support structure and a leaflet construct coupled to the support structure, as well as a tissue cutting element coupled to the support structure of the prosthetic valve, which can be referred to as an on-board cutting element. The tissue cutting element may be used in a variety of contexts, but one particular use is for cutting one or more native leaflets of a heart valve. For example, the tissue cutting element may be configured and utilized for cutting an anterior leaflet of a mitral valve to avoid left ventricular output tract (LVOT) obstruction. Though mitral valve replacement procedures are discussed herein, it will be appreciated that the scope of the disclosure also applies to other heart valves, as well as replacement of atrioventricular (“AV”) and semilunar (“SL”) valves (e.g., aortic, tricuspid, or pulmonary valves), venous valves, or other areas of the body that would benefit from selective flow, or valved flow. The disclosure should therefore not be interpreted as being limited to mitral valve replacement. Additionally, though the following examples are generally provided in the context of prosthetic valves, it should be understood that the tissue cutting elements described herein may be applied to one or more support structures associated with other implants, including stents, stent grafts, endovascular filters, occluders (e.g., septal occluders), and others.

The support structure of the prosthetic valve may include one or more frame elements or frame members (e.g., connected by an interstage component) and one or more covers. For example, the support structure may include a leaflet frame subcomponent and an optional anchor frame subcomponent, as well as an interstage between the leaflet frame subcomponent and the anchor frame subcomponent as applicable.

In various examples, the prosthetic valve is operable as a one-way prosthetic valve that defines a valve orifice into which leaflets open to permit flow and close so as to block or occlude the valve orifice and partially or entirely prevent flow in response to differential fluid pressure. Examples presented herein provide a prosthetic valve that includes a leaflet frame subcomponent, an optional anchor frame subcomponent and an associated interstage flexibly disposed between the leaflet frame subcomponent and the anchor frame subcomponent. The leaflet frame subcomponent further includes leaflets that operate as a one-way valve. The anchor frame subcomponent may be operable to couple to an implant site. The interstage is operable to permit the translation of the leaflet frame subcomponent into the anchor frame subcomponent during deployment. Further, in accordance with some embodiments, the interstage is operable to permit perfusion during deployment.

In embodiments comprising multiple leaflets, each leaflet generally cooperates with at least one neighboring or adjacently situated leaflet to block or restrict the retrograde flow of blood. The pressure differential in the blood is caused, for example, by the contraction of a ventricle or atrium of the heart, such pressure differential typically resulting from a fluid pressure building up on one side of the leaflets when closed. As the pressure on the inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve raises above the blood pressure on the inflow side of the valve, the leaflet returns to the closed position generally preventing retrograde flow of blood through the valve.

The embodiments and examples discussed herein include various apparatus, systems, and methods for a prosthetic valve, such as, but not limited to, cardiac valve replacement. In some examples, the prosthetic valve is operable as a one-way valve wherein the prosthetic valve defines a valve orifice into which leaflets open to perm it flow and close so as to occlude the valve orifice and prevent flow in response to differential fluid pressure. In the instant disclosure, the examples are primarily described in association with prosthetic valves or mechanisms of similar structure and/or function, including surgically implanted valves, although it should be readily appreciated features of such examples are equally applicable to transcatheter cardiac valve applications.

Embodiments herein include various apparatus, systems, and methods for a prosthetic valve suitable for surgical and transcatheter placement, such as, but not limited to, cardiac valve replacement. The valve is operable as a one-way valve wherein the valve defines a valve lumen into which leaflets open to permit flow and close so as to occlude the valve lumen and prevent flow in response to differential fluid pressure.

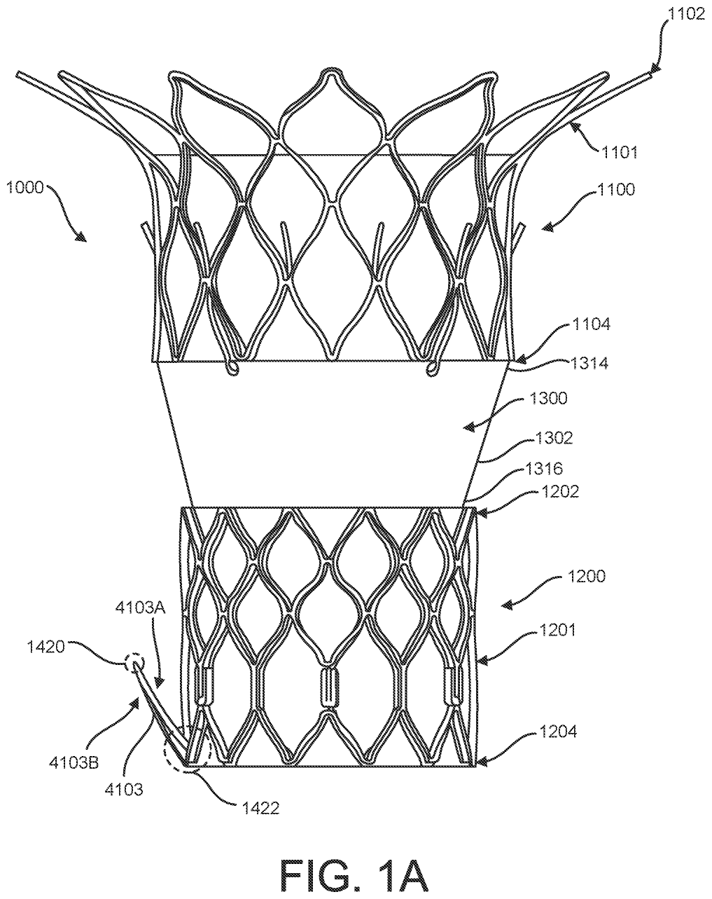

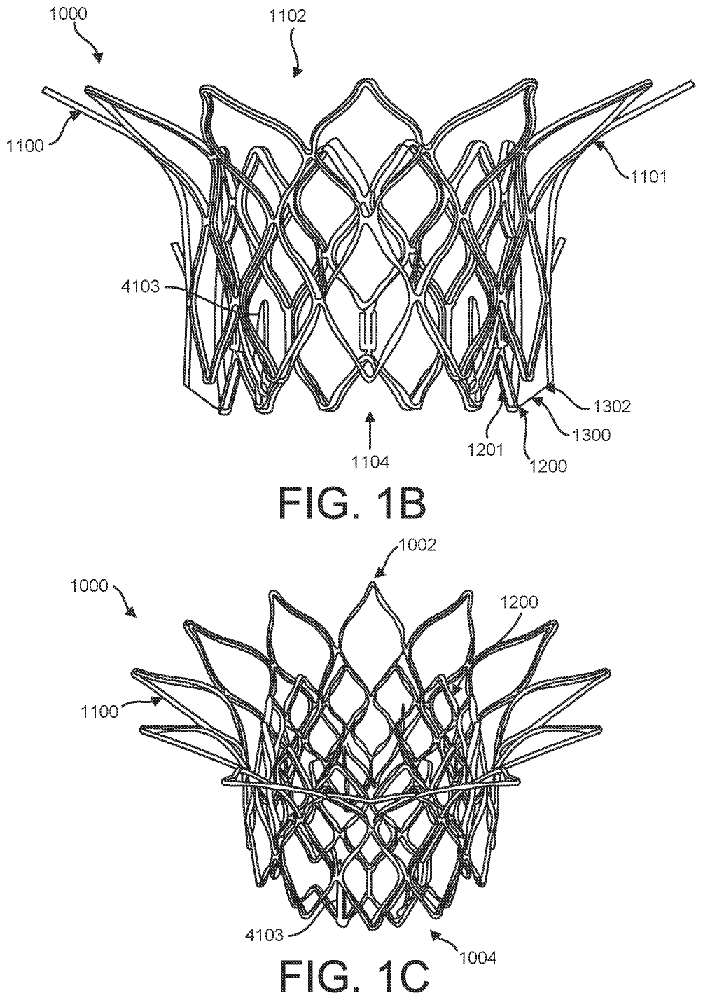

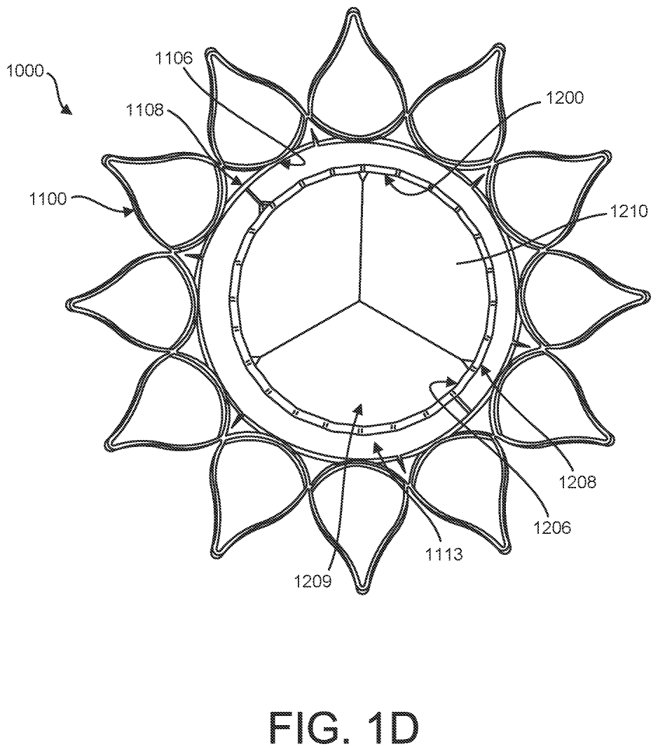

In the instant disclosure, the examples are primarily described in association with surgical or transcatheter cardiac valve applications, although it should be readily appreciated embodiments within the scope of this disclosure can be applied toward any prosthetic valve or mechanism of similar structure and/or function. For example, the prosthetic valve 1000 of FIGS. 1A through 1D can be applied in non-cardiac applications, such as vasculature, respiratory or gastrointestinal tract applications.

As will be describe further below, in various examples, the prosthetic valve provides a leaflet frame subcomponent that essentially floats within an anchor frame subcomponent supported by the interstage and does not directly couple with a native valve orifice. The anchor frame subcomponent may conform to the shape of the native valve orifice which, for example, may not be perfectly circular, whereas the leaflet frame subcomponent does not necessarily conform to the shape of the native valve orifice. The leaflet frame subcomponent may remain cylindrical or at a preferred geometrical configuration so as to present the leaflets with a geometrically stable platform ensuring proper leaflet function, including coaptation and opening dynamics.

Although it is appreciated that the examples of the prosthetic valve may be suitable for either surgical or transcatheter applications, examples provided herein are presented as for transcatheter applications to avoid the repetition if surgical examples are also presented. Therefore, the inventive concepts are applicable for both surgical or transcatheter applications and not limited to only transcatheter applications.

Various embodiments illustrated and described herein are directed to a prosthetic valve that comprises a leaflet frame subcomponent 1200 and an anchor frame subcomponent 1100 that can be nested in-situ, which is possible because an inner diameter of the anchor frame subcomponent 1100 is greater than an outer diameter of the leaflet frame subcomponent 1200. FIG. 1A is a side view of the prosthetic valve 1000 in the unrestrained configuration (e.g., unconstrained on a benchtop) showing a leaflet frame subcomponent 1200, an anchor frame subcomponent 1100, and an interstage 1300 therebetween in coaxial serial alignment. FIG. 1B is a side view of the prosthetic valve 1000 in the deployed configuration showing the leaflet frame subcomponent 1200 translated into the anchor frame subcomponent 1100, with the interstage 1300 therebetween in nested alignment.

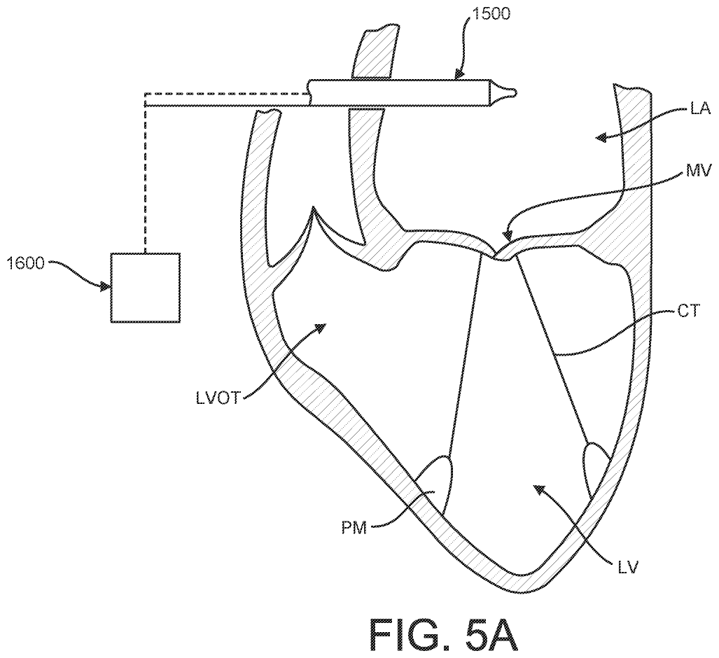

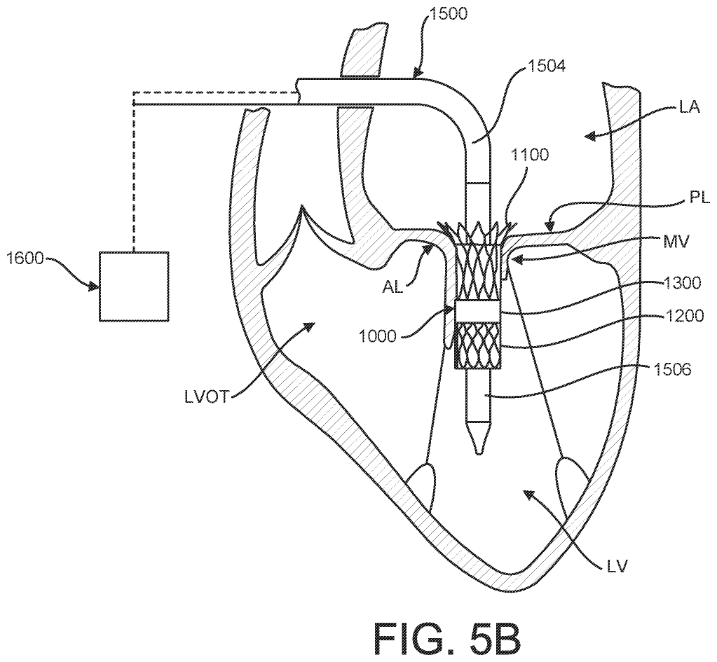

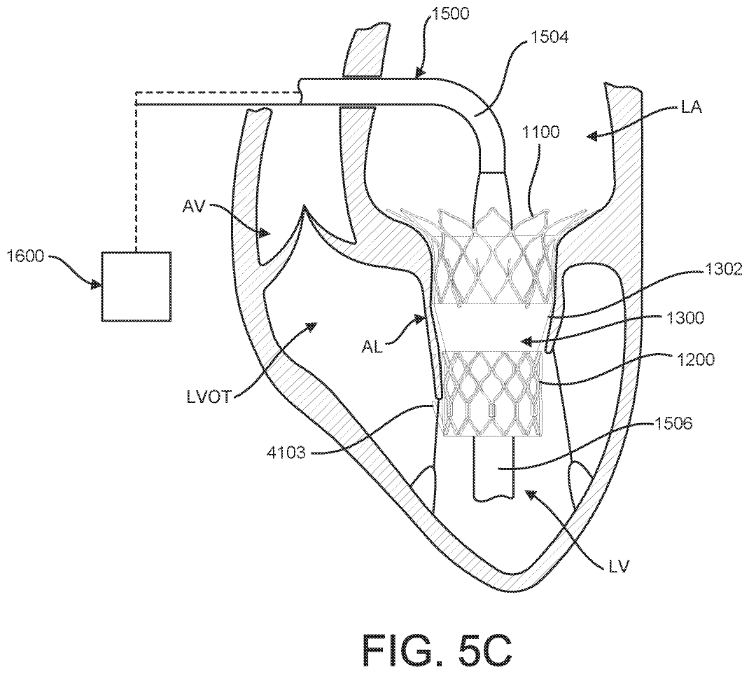

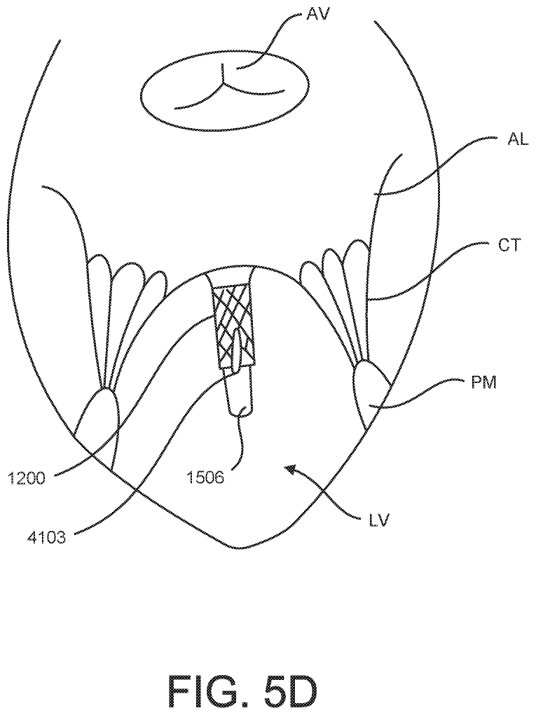

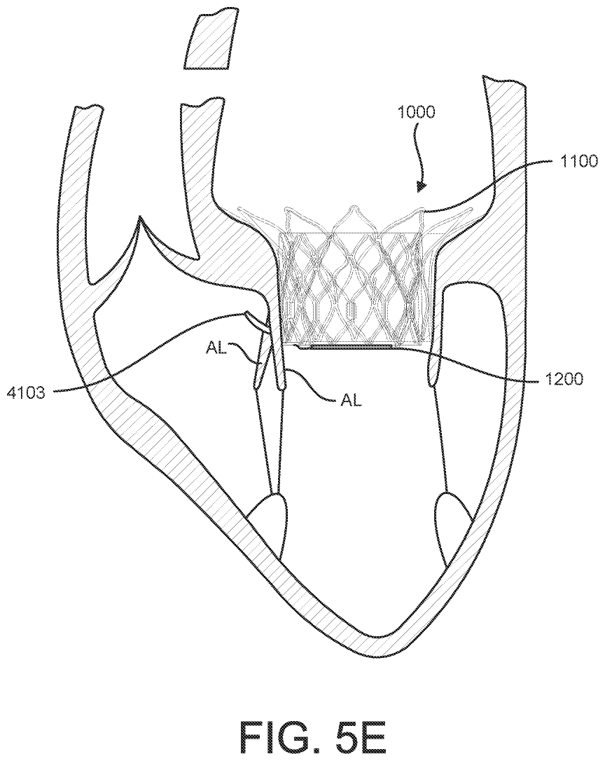

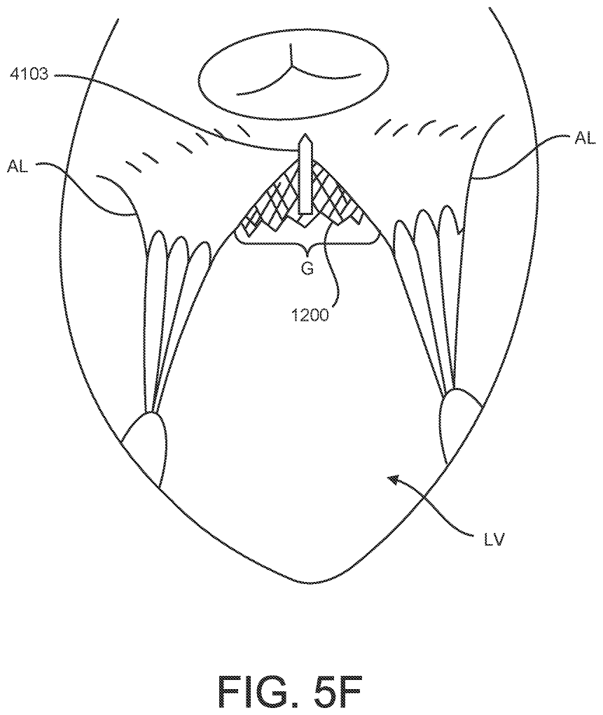

In some embodiments, the prosthetic valve 1000 is delivered into a vessel using a transcatheter approach, for example through a blood vessel within a body. A delivery device 1500 (e.g., FIG. 5A) can be used to maintain the prosthetic valve 1000 in a collapsed or compressed state before the prosthetic valve 1000 is deployed inside at a desired treatment site. As the prosthetic valve 1000 is deployed, the prosthetic valve 1000 takes on an expanded state in which the outer periphery of the prosthetic valve 1000 is greater than in the compressed state. In some examples, the prosthetic valve 1000 is a substantially circular cross-section, therefore the diameter of the prosthetic valve 1000 increases when deployed from the inside of the delivery device 1500.

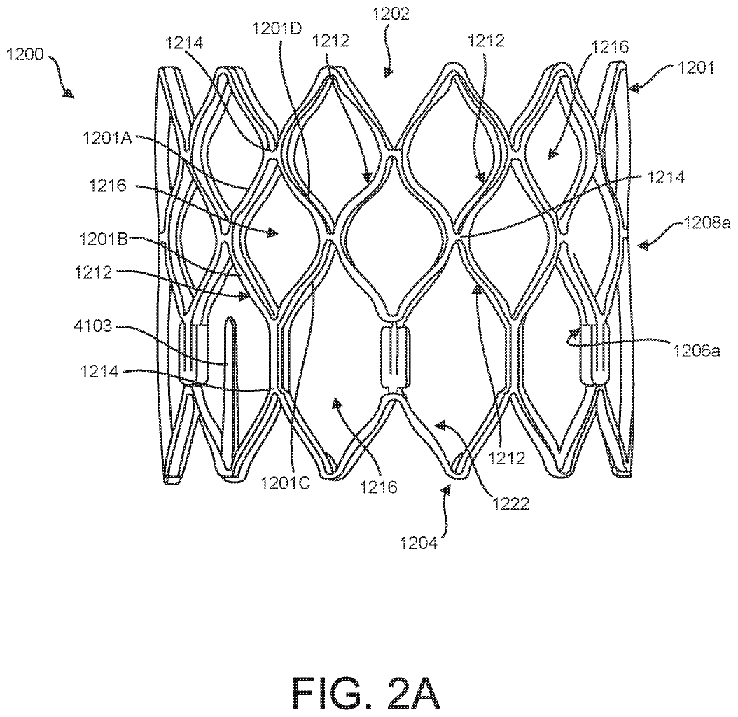

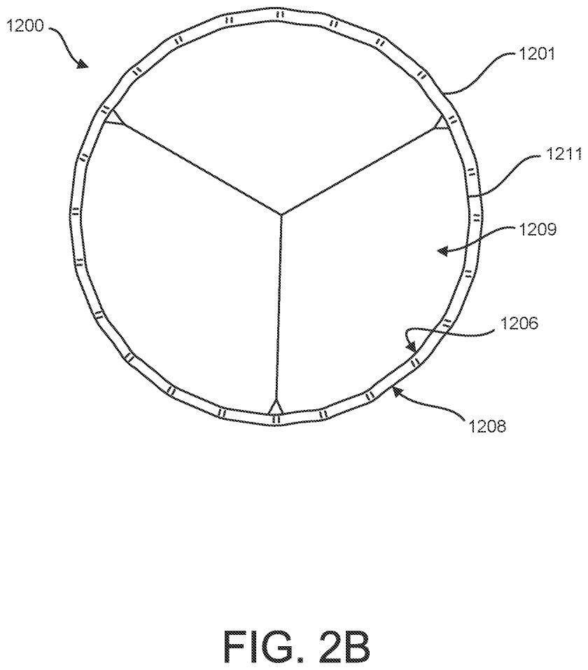

The prosthetic valve 1000 includes one or more frames, such as leaflet frame 1201 and/or anchor frame 1101 defined by a framework including a plurality of frame members. As shown in FIG. 2A, the frame members create apertures. Those apertures are generally configured to increase in area as the prosthetic valve 1000 expands. In various examples, when the prosthetic valve 1000 is deployed, the frame members can expand such that the leaflet frame 1201 may take on to a generally cylindrical shape.

The framework (e.g., leaflet frame 1201 and/or anchor frame 1101) is comprised of a plurality of frame members positioned adjacent each other to form the framework. In some examples, an aperture is defined by four frame members (e.g., frame members 1201A, 1201B, 1201C, and 1201D as shown in FIG. 2A). The frame members (such as frame members 1201A, 1201B, 1201C, and 1201D) collectively form the framework that defines a pattern of open and/or closed apertures (e.g., apertures 1216 and/or apertures 1116), one or more of which may be in a shape of a diamond or rhombus.

It is appreciated that the frame can be formed via various manufacturing processes, including, but not limited to, additive manufacturing, subtractive manufacturing, and injection molding. For example the frame may be formed of wire or braided wire, formed by three-dimensional printing, as well as formed by etching, cutting, laser cutting, and stamping sheets or tubes of material, among other suitable processes, into an annular or tubular structure or, if a sheet of material, with the sheet then formed into an annular or tubular structure. In various examples, the frame can comprise, such as, but not limited to, any biocompatible and, in those embodiments where applicable, elastically deformable metallic or polymeric material including shape-memory materials, such as nitinol, a nickel-titanium alloy. Other materials suitable for the frame include, but are not limited to, other titanium alloys, stainless steel, biocompatible alloys (e.g., cobalt-chromium alloy, cobalt-nickel alloy, nickel-cobalt-chromium alloy, or nickel-cobalt-chromium-molybdenum alloy, such as MP35N® alloy (SPS Technologies, Jenkintown, Pa.)), polymers, polypropylene, polyethylene terephthalate, PEEK, acetyl homopolymer, acetyl copolymer, other alloys, polymers, and thermoplastics, or any other material that is generally biocompatible having adequate physical and mechanical properties to function as a frame as described herein, or combinations thereof.

It is understood that when the frame is constructed of a plastically-deformable material, the frame, and thus the prosthetic valve, can have a lower radial dimension when coupled to the delivery catheter and then can be expanded to a larger radial dimension inside a patient, either by internal forces or by external forces, such as by an inflatable balloon or equivalent expansion mechanism. When constructed of an elastic material, the frame, and thus the prosthetic valve, can be crimped to a radially collapsed configuration and restrained in the collapsed configuration by, for example, insertion into a sheath or constrained by fibers or other constraining mechanism. Once inside the body, the prosthetic valve can be released from the constraining mechanism, which allows the prosthetic valve to expand to its functional size.

### Leaflet Frame Subcomponent

The leaflet frame subcomponent 1200 generally comprises a suitable material (e.g., a shape memory material operable to flex under load and retain its original shape when the load is removed),to allow the leaflet frame subcomponent 1200 to self-expand from a compressed shape to a predetermined shape, or to be expanded (e.g., balloon expanded) to a predetermined shape. Thus, the leaflet frame subcomponent 1200 may be plastically deformable to be expanded by a balloon, while in other embodiments the leaflet frame subcomponent 1200 is elastically deformable so as to be self-expanding. The leaflet frame subcomponent 1200 and the anchor frame subcomponent 1100 may comprise the same or different materials, and the description of the general features of each can readily be applied to the other.

The leaflet frame subcomponent 1200 provides the prosthetic valve 1000 with the functionality of a one-way valve. It is understood and appreciated that one-way valves are well known in the art and may be used herein. It is appreciated that mechanical valves, biological valves, and biological and synthetic leaflet valves may be used as the one-way valve of the leaflet frame subcomponent 1200. It is also appreciated that, for transcatheter applications, the leaflet frame subcomponent 1200 is required to have a smaller-diameter compressed configuration and a larger-diameter expanded configuration, and that the one-way valve component must be able to accommodate that functionality.

The leaflet frame subcomponent 1200 is configured to be received within at least a portion of the anchor frame subcomponent 1100, as will be described in more detail below. It will be appreciated that nonlimiting examples of leaflet frame subcomponent 1200 can be provided with a diameter (e.g., a diameter of an interior or exterior surface of the leaflet frame subcomponent 1200) in a range of between twenty (20) millimeters and thirty (30) millimeters, depending on a patient's anatomy, although a variety of dimensions are contemplated.

In FIGS. 1A through 1C, a tissue cutting element 4103 that is activatable, or able to be transitioned to an activated state, is shown as being functionally coupled with the leaflet frame subcomponent 1200. In FIG. 1A, the tissue cutting element 4103 is in the activated configuration, for example radially extending from an outer periphery of the leaflet frame subcomponent 1200. The tissue cutting element 4103 is operable to engage adjacent structures, e.g., tissue as will be detailed below. In some examples, the tissue cutting element 4103 is movable between an inactive position and an active position. In some embodiments, the tissue cutting element 4103 is positioned in an inactive position prior to the prosthetic valve being fully deployed. In the inactive position, as shown in FIGS. 1 B and 1C, the tissue cutting element 4103 is not operable to engage tissue or to engage tissue to the same extent as when in the active position. In the active position, the tissue cutting element 4103 extends away from the leaflet frame subcomponent 1200 to the extent necessary to engage the target tissue. In the inactive position, the tissue cutting element 4103 does not extend away from the leaflet frame subcomponent 1200 the same amount, or to the same extent, as when in the active position. In some examples, the tissue cutting element 4103 is located at least partially within the portion of the support structure from which it extends, for example within the anchor frame subcomponent 1100 or the leaflet frame subcomponent 1200.

In some examples, the tissue cutting element 4103 is operable to cut (e.g., bisect) adjacent tissue, such as a native leaflet, including an anterior leaflet of a mitral valve, when activated. As an additional example, the tissue cutting element 4103 may be operable to additionally or alternatively cut other tissue structures, such as the chordae tendineae. In some examples, the tissue cutting element 4103 is a single-edged blade, or has a single-edged configuration, although other configurations include double-edged configurations are contemplated.

FIG. 2A is a side view of the leaflet frame 1201 without leaflets 1210 or any optional covering shown for clarity. The tissue cutting element 4103 is shown in an inactive position. A number of methods and components may be utilized to maintain the tissue cutting element 4103 in this position, as further explained herein. FIG. 2B is an axial view of the leaflet frame subcomponent 1200 showing the leaflets 1210 therein. The side of the leaflet frame 1201 may be at least partially covered, such as with a film or fabric, not shown for clarity, suitable for a particular purpose, such as, but not limited to, to restrict fluid from passing through apertures of the leaflet frame 1201. For illustrative purposes, the following examples are suitable especially fora transcatheter application, but are also suitable for a surgical application. The leaflet frame subcomponent 1200 includes a leaflet frame 1201 and leaflets 1210.

The leaflet frame 1201 defines a cylindrical or tubular mesh or framework defining the apertures 1216, in accordance with an embodiment. For example, as shown, the leaflet frame 1201 includes a plurality of frame members 1212 that are interconnected and arranged in one or more patterns. In various examples, the frame members 1212 are connected to one another at joints 1214. In some examples, these joints 1214 operate as flex points so as to provide a preferential flexing location for the leaflet frame subcomponent 1200, such as to flex when compressed to a smaller delivery diameter such as required for transcatheter delivery. In some examples, one or more flex points or joints 1214 comprise sites on the leaflet frame 1201 that undergo a high degree of bending. In some examples, the flex points or joints 1214 may comprise a geometry, structural modification or material property modification, among others, that biases the leaflet frame 1201 to bend at the joints 1214 when compressed or expanded between a larger diameter and a smaller diameter.

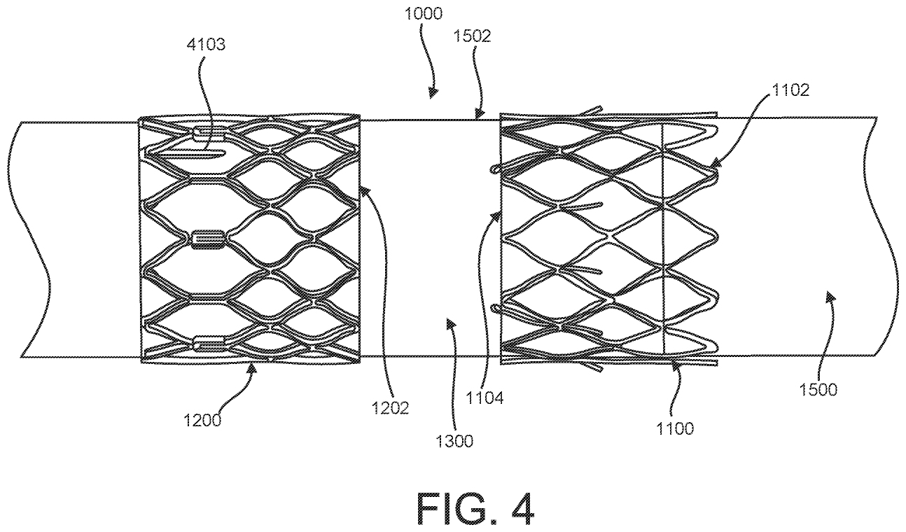

In some examples, one or more apertures 1216 (also described as cells) are defined between the joints 1214 and the frame members 1212 of the leaflet frame 1201 that are interconnected. In instances where the apertures 1216 are bounded on all sides by frame members 1212 or other features, they may be referred to as “closed” apertures and where not bounded on all sides, “open” apertures. In some examples, these apertures 1216 extend between the leaflet frame exterior surface 1208a and the leaflet frame interior surface 1206a of the leaflet frame subcomponent 1200. As illustrated in the embodiments of FIGS. 2A and 2B, one or more of the apertures 1216 define a diamond shape when the leaflet frame subcomponent 1200 is in a deployed configuration. Upon compression to a smaller diameter (e.g., a delivery diameter), one or more of the joints 1214 and the frame members 1212 deform such that the apertures 1216 generally define an elongated diamond shape (e.g., as shown generally in FIG. 5D). Upon re-expanding the leaflet frame subcomponent 1200 to a larger diameter during deployment at a treatment site, the frame members 1212 that are interconnected expand to define the generally wider diamond shape and thus apertures 1216 that are generally wider (e.g., as shown generally in FIG. 4).

It should be appreciated that while the frame members 1212 illustrated and described herein are interconnected and define apertures 1216 having generally a diamond shape, the frame members 1212 that are interconnected may be arranged in a number of alternative patterns without departing from the spirit or scope of the disclosure. That is, a number of alternative patterns are envisioned where the arrangement of frame members 1212 is configured in such a manner as to provide for a leaflet frame subcomponent 1200 that can be compressed to a smaller diameter for transcatheter delivery and subsequently expanded (or allowed to expand) to a larger diameter at a treatment site during deployment of the prosthetic valve 1000. Accordingly, the disclosure should not be limited to arrangements of the frame members 1212 that define apertures 1216 that are diamond-shaped. For example, a framework of the leaflet frame subcomponent 1200 can define any number of features, repeatable or otherwise, such as geometric shapes and/or linear or meandering series of sinusoids. Geometric shapes can comprise any shape that facilitates circumferential compressibility and expandability.

In various embodiments, the anchor frame 1101 and/or the leaflet frame 1201 may comprise or otherwise be formed from a cut tube, or any other element suitable for the particular purpose of the frame as described herein. In various examples, the anchor frame 1101 and/or the leaflet frame 1201 is elastically deformable so as to be self-expanding under spring bias forces, as those of skill will appreciate. In some examples, the frame is plastically deformable so as to be mechanically expanded such as with a balloon, as those of skill will appreciate. In yet some other examples, the frame is plastically deformable as well as elastically deformable. That is, in some examples, the frame includes one or more elastically deformable components or features and one or more plastically deformable components or features. Thus, it should be appreciated that the examples of the frame presented herein are not to be limited to a specific design or mode of expansion.

The anchor frame 1101 and/or the leaflet frame 1201 can be made of any of various suitable plastically-expandable materials (e.g., stainless steel, etc.) or self-expanding materials (e.g., nickel titanium alloy (NiTi), such as nitinol), in accordance with embodiments. When constructed of a plastically-expandable material, the frame, and thus the prosthetic valve 1000, can be compressed to a radially collapsed configuration in the delivery device 1500 and then expanded inside a patient by an inflatable balloon or equivalent expansion mechanism (not shown). When constructed of a self-expandable material, the frame, and thus the prosthetic valve 1000, can be crimped to a radially collapsed configuration and restrained in the collapsed configuration by insertion into a sheath or equivalent mechanism of the delivery device 1500. Once inside the body, the prosthetic valve 1000 can be advanced from the delivery sheath which allows the prosthetic valve to expand to its functional size.

Suitable plastically-expandable materials that can be used to form the frame include, without limitation, stainless steel, biocompatible alloys (e.g., cobalt-chromium or nickel-cobalt-chromium alloys), polymers, or combinations thereof. In some embodiments, the frame is made of a nickel-cobalt-chromium-molybdenum alloys, such as MP35N® alloy (SPS Technologies, Jenkintown, Pa.).

In some methods of making the leaflet frame 1201, a pattern of the apertures 1216 may be cut into a tube to form an annular-shaped cut stent frame or support structure defining the leaflet frame 1201. As shown, the apertures 1216 may generally be arranged into rows forming the annular shape of the support structure. The leaflet frame 1201 may be formed or otherwise configured such that the apertures 1216 have differing sizes and/or configurations (e.g., one or more rows of smaller apertures and one or more rows of larger apertures as shown).

In various embodiments, the leaflet frame subcomponent 1200 additionally supports or otherwise includes a valve structure. In some examples, the valve structure includes one or more leaflets 1210 as shown in FIG. 1D. A variety of mechanical valve leaflet, biological leaflet, and synthetic leaflet designs are known in the medical technology arts, any of which may be incorporated into the leaflet frame subcomponent 1200 of the present disclosure.

In some examples, the leaflets 1210 are coupled to the leaflet frame subcomponent interior surface 1206 (e.g., to the leaflet frame interior surface 1206a). The leaflets 1210 may be coupled to the leaflet frame subcomponent 1200 in any of a variety of manners, including by wrapping one or more portions of the leaflets about one or more portions of the leaflet frame 1201, by suturing or sewing one or more portions to the leaflet frame 1201, but using adhesives or other mechanical fasteners, or by using one or more projections on the leaflet frame 1201 (not shown) that pass through one more apertures configured to be disposed about the one or more projections (not shown). Any of these coupling mechanisms, combinations thereof, and others may be employed as appropriate.

### Interstage

Referring to FIG. 1A, the interstage 1300 includes a conduit 1302 that is coupled to an anchor frame subcomponent outlet end 1104 of the anchor frame subcomponent 1100 at an interstage first end 1314 and is coupled to a leaflet frame subcomponent inlet end 1202 at an interstage second end 1316. The conduit 1302 may comprise any suitable material known in the art that is flexible and is operable to be everted as discussed below. By way of example, the conduit 1302 may be a film or fabric, among others.

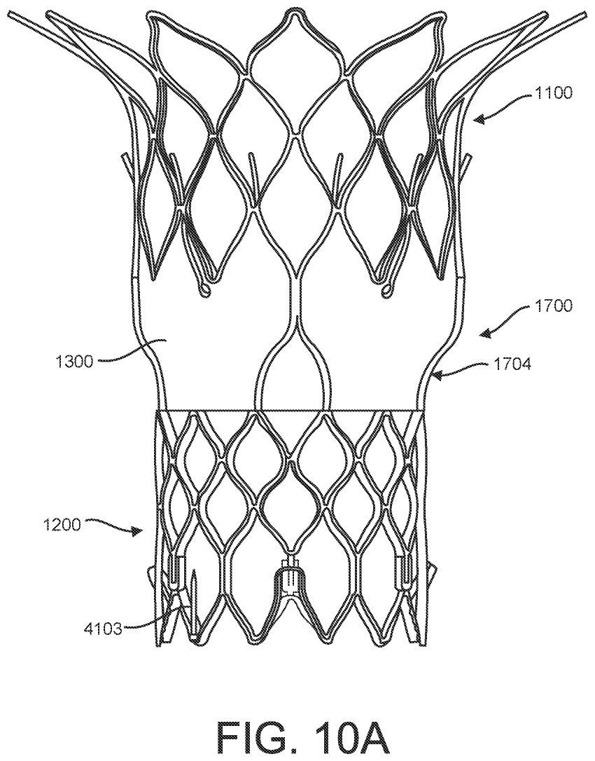

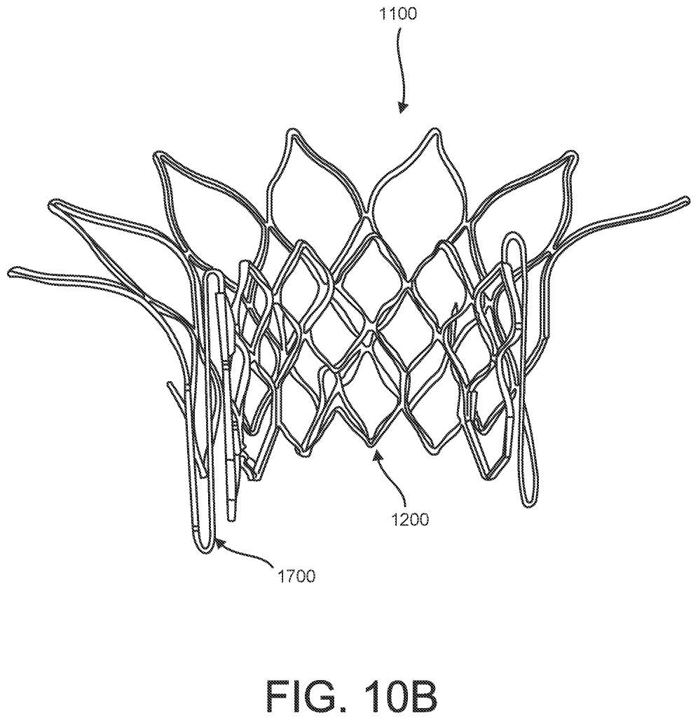

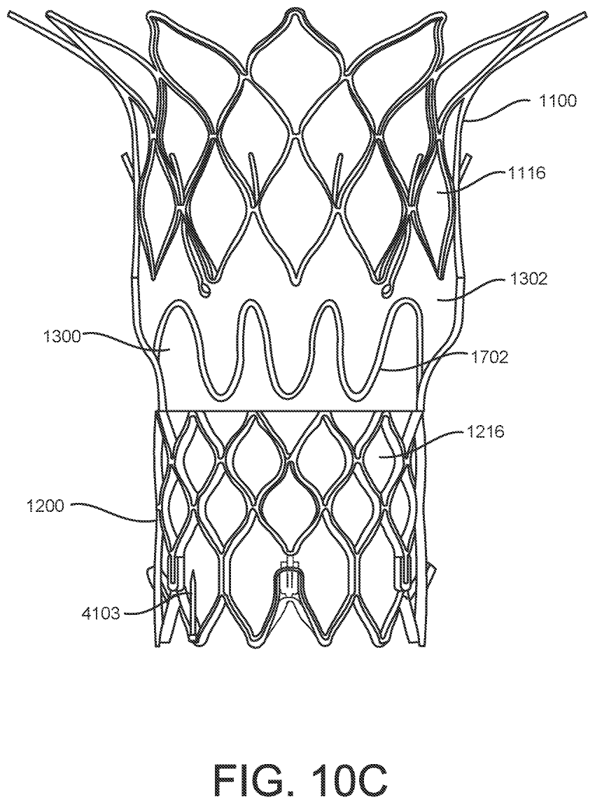

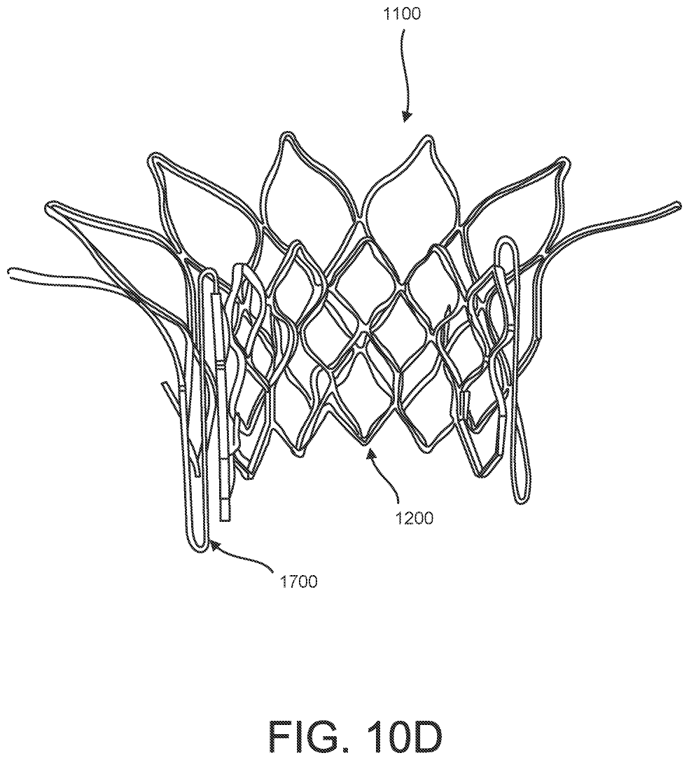

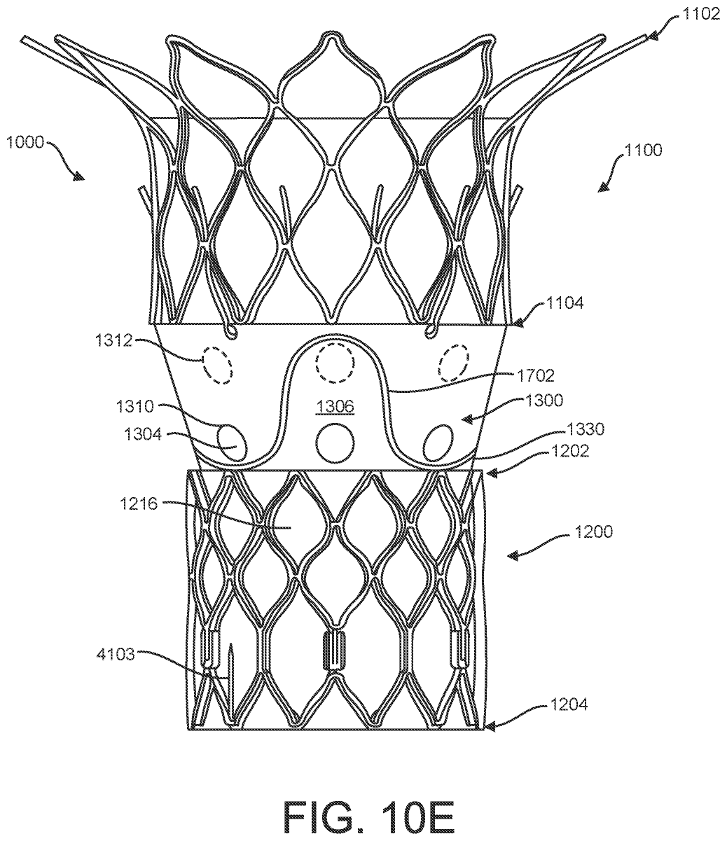

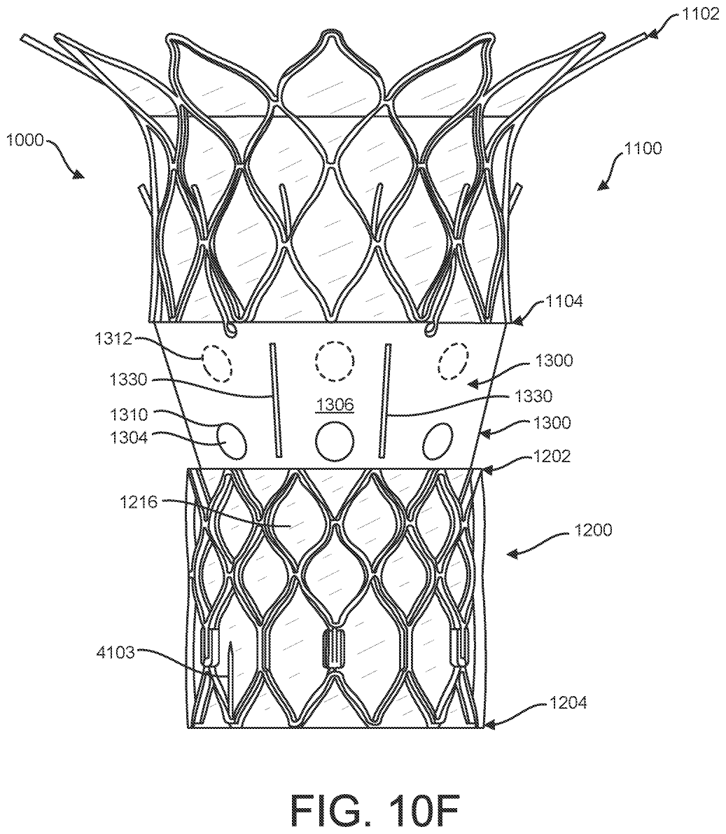

In various examples, the interstage 1300 further comprises one or more nesting retention elements 1330, such as shown in FIGS. 10E and 10F, that is operable to retain the position of the leaflet frame subcomponent 1200 as nested in the anchor frame subcomponent 1100. For example, the leaflet frame subcomponent 1200 may be nested in the anchor frame subcomponent 1100 in a substantially fixed relationship during operation, or may be allowed to flex or displace in operation (e.g., axially and/or transversely), but to generally return to a neutral, desired position.

In accordance with some examples, the nesting retention elements 1330 may be one or more elongated elements that bias the interstage 1300 in the nested position, particularly after the leaflet frame subcomponent 1200 is expanded. For example, in FIG. 10E, the nesting retention elements 1330 may have a serpentine or sinuous configuration, whereas in FIG. 10F, the nesting retention elements 1330 may be straight and generally aligned with the longitudinal axis of the prosthetic valve 1000. In some examples, the nesting retention elements 1330 can be positioned between the locations of two neighboring ones of the inner apertures 1312 and/or between two neighboring ones of the outer apertures 1310 of the interstage 1300. In accordance with an embodiment, the nesting retention elements 1330 are caused to evert during the deployment process of translating the leaflet frame subcomponent 1200 as compressed into the anchor frame subcomponent 1100. The nesting retention elements 1330 are provided with a predetermined stiffness or other property sufficient to permit eversion during deployment when the leaflet frame subcomponent 1200 is compressed but not under normal biological forces when the leaflet frame subcomponent 1200 is expanded. In accordance with another embodiment, the nesting retention elements 1330 are sized such that, when the anchor frame subcomponent 1100 is expanded and the leaflet frame subcomponent is compressed, the nesting retention elements 1330 are able to rotate lengthwise from a forward-facing orientation to a backward facing orientation. When the leaflet frame subcomponent 1200 is expanded, the nesting retention elements 1330 have a profile or length that prevents the nesting retention elements 1330 from rotating or flipping back to a forward-facing orientation. In other words, the gap between the anchor frame subcomponent 1100 and the leaflet frame subcomponent 1200 which is nested therein is too narrow to allow end over end rotation of the nesting retention elements 1330. The nesting retention elements 1330 are provided with a predetermined stiffness or other property sufficient to prevent eversion of the nesting retention elements 1330 within the gap between the anchor frame subcomponent 1100 and the leaflet frame subcomponent 1200 under expected biological forces.

FIG. 1C is a perspective view showing the leaflet frame subcomponent 1200 and an anchor frame subcomponent 1100 of a prosthetic valve 1000 in a nested configuration, also referred to as the deployed position, leaflets not shown for clarity. FIG. 1B is a front view of the leaflet frame subcomponent 1200 and the anchor frame subcomponent 1100 of the prosthetic valve 1000 of FIG. 1C. In both FIGS. 1B and 1C, the leaflets and any film, as will be discussed below, are not shown for clarity. FIG. 1D is an axial view of the leaflet frame subcomponent 1200 and the anchor frame subcomponent 1100 of the prosthetic valve 1000 of FIG. 1A, showing the leaflets 1210. In the axial view of FIG. 1D, three leaflets 1210 are shown coupled to the leaflet frame subcomponent 1200. It is in this deployed position that the prosthetic valve 1000 remains in the native valve orifice to function as a prosthetic valve. The anchor frame subcomponent 1100 and the leaflet frame subcomponent 1200 are longitudinally offset and generally coaxial relative to one another.

With continued reference to FIGS. 1A to 1D, the prosthetic valve 1000 includes the anchor frame subcomponent 1100, and the leaflet frame subcomponent 1200. In the deployed configuration, the leaflet frame subcomponent 1200, which includes the leaflets 1210, is positioned at least partially within the anchor frame lumen 1113 (FIG. 1D) of the anchor frame subcomponent 1100. The prosthetic valve 1000 has a prosthetic valve inlet end (e.g., corresponding to anchor frame subcomponent inlet end 1102 and/or leaflet frame subcomponent inlet end 1202) and a prosthetic valve outlet end (e.g., corresponding to anchor frame subcomponent outlet end 1104 and/or leaflet frame subcomponent outlet end 1204). In various examples, when deployed within the body, the prosthetic valve inlet end (e.g., corresponding to anchor frame subcomponent inlet end 1102 and/or leaflet frame subcomponent inlet end 1202) is positioned upstream relative to the prosthetic valve outlet end, which is positioned downstream relative to the prosthetic valve inlet end (e.g., corresponding to anchor frame subcomponent inlet end 1102 and/or leaflet frame subcomponent inlet end 1202).

In various embodiments, the anchor frame subcomponent 1100 and the leaflet frame subcomponent 1200 are coupled together by the interstage 1300. Referring to FIG. 4, showing a side view of the prosthetic valve 1000 in a pre-deployed configuration on delivery device 1500, in some examples, the interstage 1300 is disposed between the anchor frame subcomponent 1100 and the leaflet frame subcomponent 1200 coupling them together. In some examples, the interstage 1300 includes a portion of a contiguous film that extends over a portion of the anchor frame subcomponent 1100 and the leaflet frame subcomponent 1200 operable to couple them together. In some examples, the contiguous film extends not only between but also over or within either or both of the anchor frame subcomponent 1100 and the leaflet frame subcomponent 1200. In some examples, the interstage 1300 is formed from a generally tubular material. In some examples, the interstage 1300 is formed by wrapping a film over and around a cylindrical mandrel, with either or both of the anchor frame 1101 and the leaflet frame 1201 being slid over and bonded thereto to the inner surface of the frames, with the film becoming an element of the anchor frame subcomponent 1100, the leaflet frame subcomponent 1200, and the interstage 1300, respectively. In some examples, the interstage 1300 is formed by wrapping the film over and around either or both of the anchor frame 1101 and the leaflet frame 1201 and the gap therebetween and bonded thereto to the outer surface of the frames, with the film becoming an element of the anchor frame subcomponent 1100, the leaflet frame subcomponent 1200, and the interstage 1300, respectively.

In examples where the anchor frame 1101 and the leaflet frame 1201 are comprised of metal, there is a metal to polymer to metal interconnection by way of the film, wherein there is no metal to metal contact between the two frames.

The interstage 1300 is generally any material that is biologically compatible and configured to couple to the anchor frame subcomponent 1100 and the leaflet frame subcomponent 1200. In various examples, the biocompatible material is a film that is not of a biological source and that is sufficiently flexible and strong for the particular purpose, such as a biocompatible polymer. In other examples the interstage 1300 may be of biological tissue, e.g. a dehydrated bovine tissue. In an embodiment, the film comprises a biocompatible polymer (e.g., ePTFE). In some examples, the film is a composite of two or more materials. The film may comprise one or more of a membrane, composite material, or laminate.

In various examples, a prosthetic valve and its associated delivery system are configured to facilitate continued valve functionality during the deployment procedure. In various examples, during a prosthetic valve deployment procedure to replace a damaged valve, the valve and valve orifice are temporarily obstructed by the prosthetic valve and the delivery device. In some instances, such obstructions occur prior to the prosthetic valve being deployed and becoming operational (e.g., prior to nesting the anchor frame subcomponent and the leaflet frame subcomponent). Accordingly, in various examples, the prosthetic valves of the present disclosure may additionally include one or more features that are configured to permit fluid to flow through or around the prosthetic valve during the implantation procedure, prior to the prosthetic valve becoming fully operational (e.g., prior to nesting the anchor frame subcomponent and the leaflet frame subcomponent).

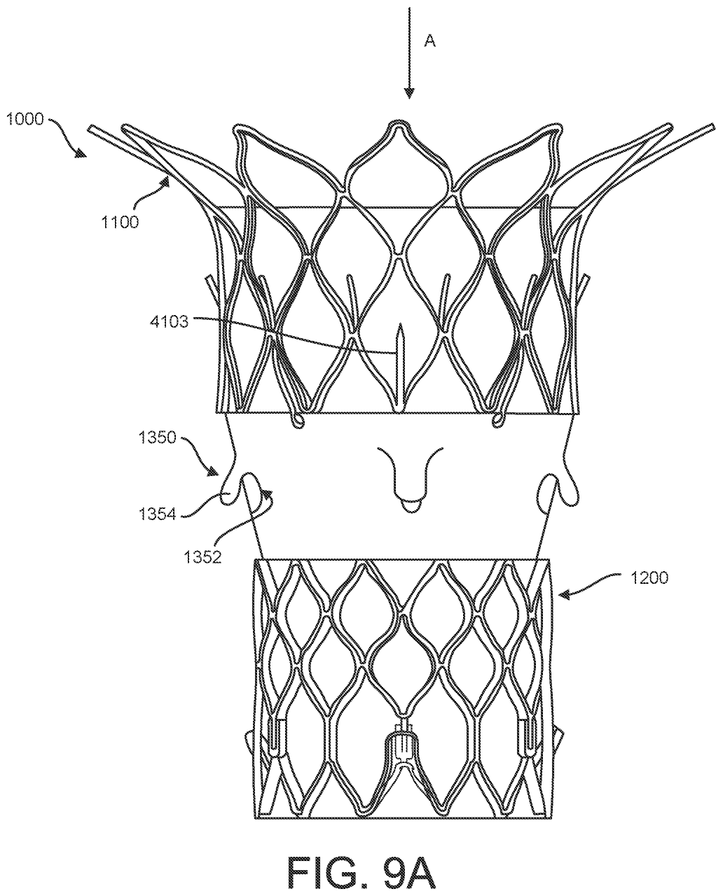

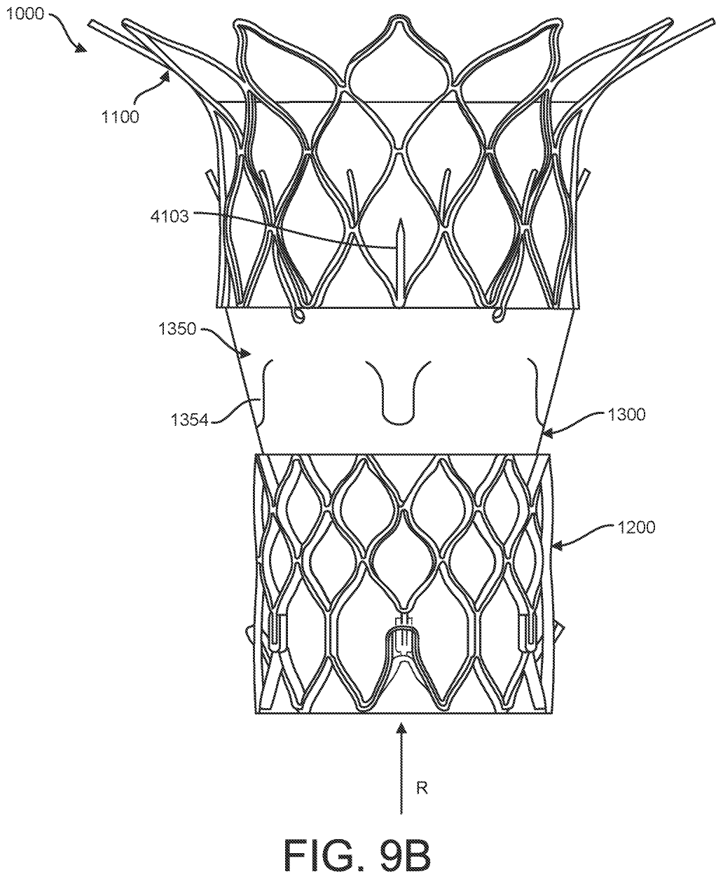

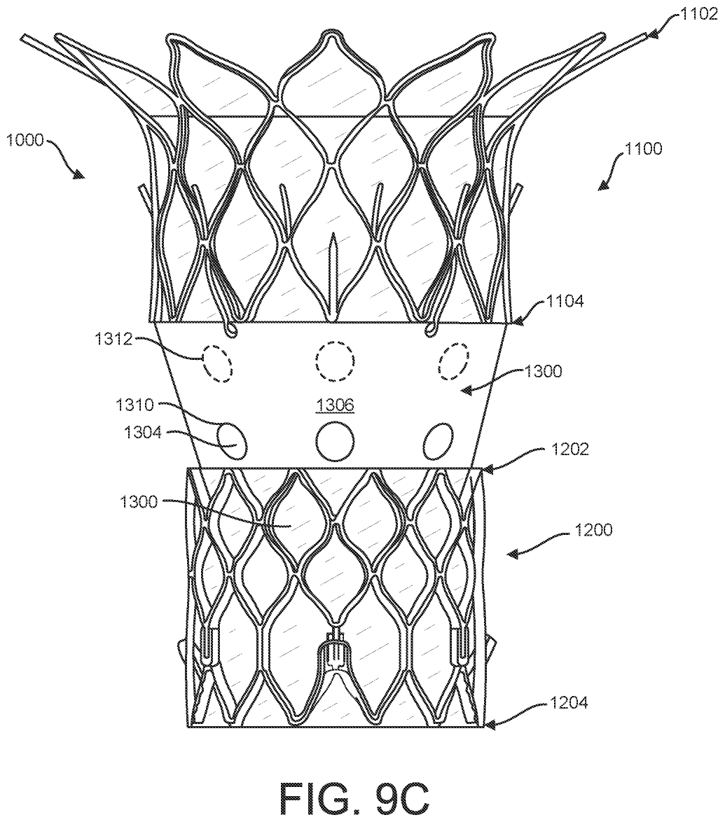

For example, and with reference to FIGS. 9A and 9B, the prosthetic valve 1000 includes one or more flow enabling features 1350 formed in the interstage 1300 extending between the anchor frame subcomponent 1100 and the leaflet frame subcomponent 1200. FIG. 9A is a side view of the prosthetic valve 2000 with the flow enabling features 1350 in an open configuration where antegrade flow (denoted by arrow “A”) is permitted. FIG. 9B is a side view of the prosthetic valve 2000 with the flow enabling features 1350 in a closed configuration where retrograde (denoted by arrow “R”) flow is obstructed. In some examples, the one or more flow enabling features 1350 include one or more perforations or apertures.

In some examples, the one or more flow enabling features 1350 additionally or alternatively include one or more mechanisms that facilitate unidirectional flow. For instance, in some examples, the flow enabling features are configured as one-way valves. In some examples, one-way valves include an aperture or perforation and a flap or element of material that overlays and is slightly larger than the aperture or perforation. In some examples, the one-way valve is oriented to permit antegrade flow through the prosthetic valve, while minimizing or preventing retrograde flow through the prosthetic valve.

As shown in FIGS. 9A and 9B, the flow enabling features 1350 include an aperture 1352 and a flap 1354 that operate to facilitate antegrade flow through the prosthetic valve 2000 prior to the anchor frame subcomponent 1100 and the leaflet frame subcomponent 1200 being nested together (i.e., while the anchor frame subcomponent 1100 and the leaflet frame subcomponent 1200 are longitudinally offset as illustrated and described herein). The flap 1354 is oversized relative to the aperture 1352 to restrict or minimize retrograde flow through the one or more flow enabling features 1350 while permitting antegrade flow

FIG. 9C is another embodiment of the interstage 1300 as shown coupled to the leaflet frame subcomponent 1200 and anchor frame subcomponent 1100. In accordance with this embodiment, the conduit 1302 of the interstage 1300 includes a double layer construct, including an inner layer 1304 that defines an inner surface of the interstage 1300 and an outer layer 1306 that defines an outer surface of the interstage 1300 as viewed in the partially deployed position. The inner layer 1304 and the outer layer 1306 are coupled together at least at the leaflet frame subcomponent inlet end 1202 of the leaflet frame subcomponent 1200 and the anchor frame subcomponent outlet end 1104 of the anchor frame subcomponent 1100. The inner layer 1304 defines one or more inner apertures 1312 therethrough adjacent the anchor frame subcomponent 1100 and the outer layer 1306 defines one or more outer apertures 1310 therethrough adjacent the leaflet frame subcomponent 1200. The inner layer 1304 and the outer layer 1306 are not coupled at least between one of the inner apertures 1312 and one of the outer apertures 1310 so as to define a flow space 1320 therebetween.

In some examples, the prosthetic valve 1000 additionally or alternatively includes one or more features that extend between the anchor frame subcomponent 1100 and the leaflet frame subcomponent 1200. For example, as shown in FIGS. 10A and 10B, the prosthetic valve 1000 includes a plurality of interconnecting struts 1700 that extend between the anchor frame subcomponent 1100 and the leaflet frame subcomponent 1200. FIG. 10A shows the prosthetic valve 1000 prior to telescoping or nesting of the anchor frame subcomponent 1100 and the leaflet frame subcomponent 1200. FIG. 10B shows the prosthetic valve 1000 with the anchor frame subcomponent 1100 and the leaflet frame subcomponent 1200 in a nested configuration.

As shown in FIGS. 10A and 10B, the interconnecting struts 1700 are configured to evert along with the interstage 1300 as the leaflet frame subcomponent 1200 is telescoped or nested with the anchor frame subcomponent 1100. In various examples, the interconnecting struts 1700 are elongate elements 1704 that are curved or s-shaped. It is appreciated that such a configuration provides that the interconnecting struts 1700 can be temporarily bent or folded upon themselves as the anchor frame subcomponent 1100 and the leaflet frame subcomponent 1200 are nested.

The interconnecting struts 1700 provide stiffening bias such that it takes a predetermined amount of force to nest the leaflet frame subcomponent 1200 into the anchor frame subcomponent 1100 and a corresponding predetermined amount of force to resist the movement of the leaflet frame subcomponent 1200 from the nested position, especially considering an interstage 1300 that does not provide sufficient resistance from movement of the leaflet frame subcomponent 1200 from the nested position. The interconnecting struts 1700 also provides a predetermined amount of lateral and radial stiffness to facilitate handling and deployment dynamics, especially considering an interstage 1300 that does not provide sufficient stiffness to facilitate from handling and deployment dynamics. In various examples, the interstage 1300 is very thin and thus provides little to no radial or lateral stiffness to resist the movement of the leaflet frame subcomponent 1200 from the nested position and/or to facilitate handling and deployment dynamics. In accordance with various examples, the interconnecting struts 1700 may be coupled to the interstage 1300, either on an inner surface, an outer surface or, in the examples having interstage 1300 that has a conduit 1302 including a double layer construct, contained between the inner layer 1304 and the outer layer 1306.

In various examples, the interconnecting struts 1700 as everted operate to maintain the nested configuration of the anchor frame subcomponent 1100 and the leaflet frame subcomponent 1200. In some examples, with the anchor frame subcomponent 1100 and the leaflet frame subcomponent 1200 in the nested configuration and the interconnecting struts 1700 everted, a column strength of the interconnecting struts 1700 operates to resist compressive loads that would otherwise cause the leaflet frame subcomponent 1200 to de-nest or telescope out of and away from the anchor frame subcomponent 1100.

In accordance with other examples, as shown in FIGS. 10C through 10E, the prosthetic valve 1000 includes one or more nesting retention elements 1330 in the form of a sinuous element 1702 (e.g., a continuously extending sinuous member) that extends between the anchor frame subcomponent 1100 and the leaflet frame subcomponent 1200 but does not couple directly therewith. The sinuous element 1702 provides stiffening bias to the interstage 1300. FIG. 10C shows the prosthetic valve 1000 prior to telescoping or nesting of the anchor frame subcomponent 1100 and the leaflet frame subcomponent 1200.

FIG. 10D shows the prosthetic valve 1000 with the anchor frame subcomponent 1100 and the leaflet frame subcomponent 1200 in a nested configuration. As shown in FIGS. 10C and 10D, the sinuous element 1702 is configured to evert along with the interstage 1300 as the leaflet frame subcomponent 1200 is telescoped or nested with the anchor frame subcomponent 1100. In various examples, the sinuous element 1702 is an elongate element that is curved or s-shaped. It is appreciated that such a configuration provides that the sinuous element 1702 can be temporarily elastically bent or folded upon itself as the anchor frame subcomponent 1100 and the leaflet frame subcomponent 1200 are nested. The sinuous element 1702 provides stiffening bias such that it takes a predetermined amount of force to nest the leaflet frame subcomponent 1200 into the anchor frame subcomponent 1100 and a corresponding predetermined amount of force to resist the movement of the leaflet frame subcomponent 1200 from the nested position, especially considering an interstage 1300 that does not provide sufficient resistance from movement of the leaflet frame subcomponent 1200 from the nested position.

The sinuous element 1702 also provides a predetermined amount of lateral and radial stiffness to facilitate handling and deployment dynamics, especially considering an interstage 1300 that does not provide sufficient stiffness to facilitate from handling and deployment dynamics. In various examples, the interstage 1300 is very thin and thus provides little to no radial or lateral stiffness to resist the movement of the leaflet frame subcomponent 1200 from the nested position and/or to facilitate handling and deployment dynamics. In accordance with various examples, the sinuous element 1702 may be coupled to the interstage 1300, either on an inner surface, an outer surface or, in the examples having an interstage 1300 with a conduit 1302 that is defined by a double layer (e.g., a double layer of film), the sinuous element 1702 may be contained between the inner layer 1304 and the outer layer 1306.

In various examples, the sinuous element 1702 as everted operates to maintain the nested configuration of the anchor frame subcomponent 1100 and the leaflet frame subcomponent 1200. In some examples, with the anchor frame subcomponent 1100 and the leaflet frame subcomponent 1200 in the nested configuration, a column strength of the sinuous element 1702 operates to resist compressive loads that would otherwise cause the leaflet frame subcomponent 1200 to de-nest or telescope out of and away from the anchor frame subcomponent 1100.

As explained, the nesting retention elements 1330 may be operable to retain the leaflet frame subcomponent 1200 as nested in the anchor frame subcomponent 1100. Examples of nesting retention elements 1330 are provided below. In accordance with some examples, the nesting retention elements 1330 may be elongated elements that bias the interstage 1300 in the nested position. In accordance with an embodiment, the nesting retention elements 1330 are caused to evert during the deployment process of translating the leaflet frame subcomponent 1200 into the anchor frame subcomponent 1100. The nesting retention elements 1330 are provided with a predetermined stiffness or other property sufficient to permit eversion during deployment but not under normal biological forces.

In accordance with another embodiment, the nesting retention elements 1330 are sized such that, when the anchor frame subcomponent 1100 is expanded and the leaflet frame subcomponent is compressed, the nesting retention elements 1330 are able to rotate lengthwise from a forward-facing orientation to a backward facing orientation. When the leaflet frame subcomponent 1200 is expanded, the nesting retention elements 1330 have a profile or length that prevents the nesting retention elements 1330 from rotating or flipping back to a forward-facing orientation. In other words, the gap between the anchor frame subcomponent 1100 and the leaflet frame subcomponent 1200 is too narrow to allow end over end rotation of the nesting retention elements 1330. The nesting retention elements 1330 are provided with a predetermined stiffness or other property sufficient to prevent eversion of the nesting retention elements 1330 within the gap between the anchor frame subcomponent 1100 and the leaflet frame subcomponent 1200 under normal biological forces. In various examples, the tissue cutting element 4103 can configured to be lodged between the anchor frame subcomponent 1100 and the leaflet frame subcomponent 1200 when the leaflet frame subcomponent 1200 as nested in the anchor frame subcomponent 1100 so as to prevent the tissue cutting element 4103 from engaging with the adjacent tissue surface while the prosthetic valve 1000 is in the nested configuration.

### Anchor Frame Subcomponent

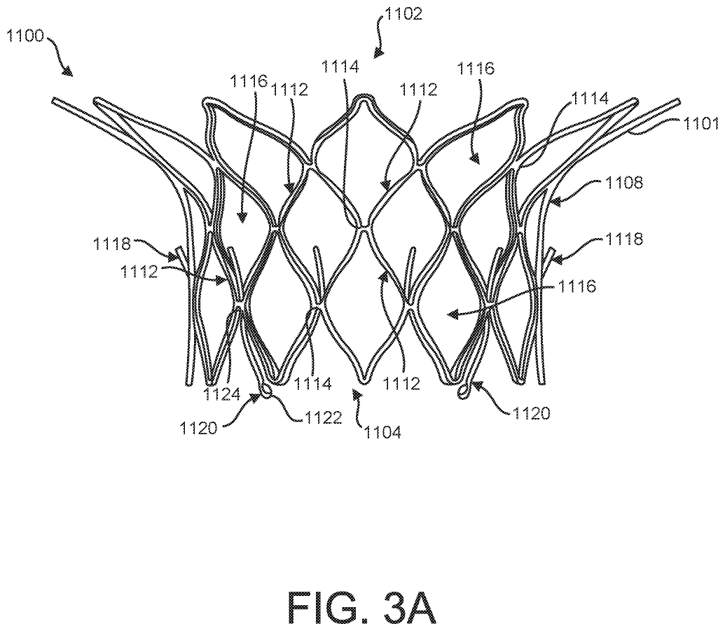

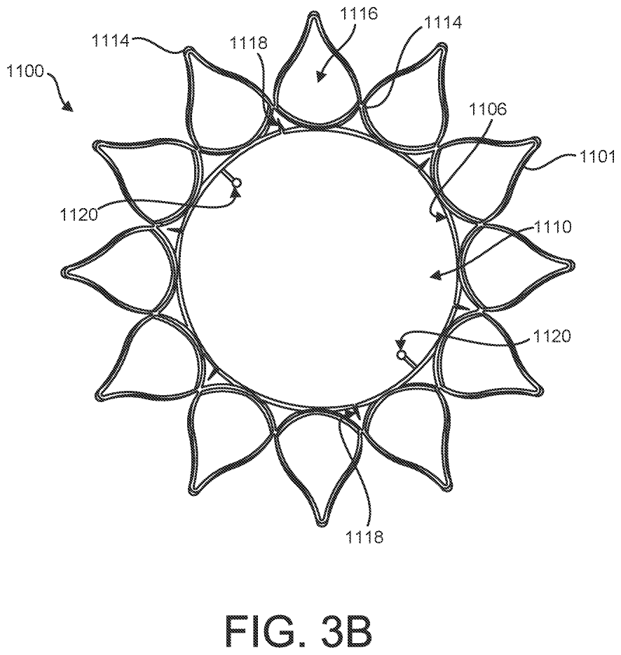

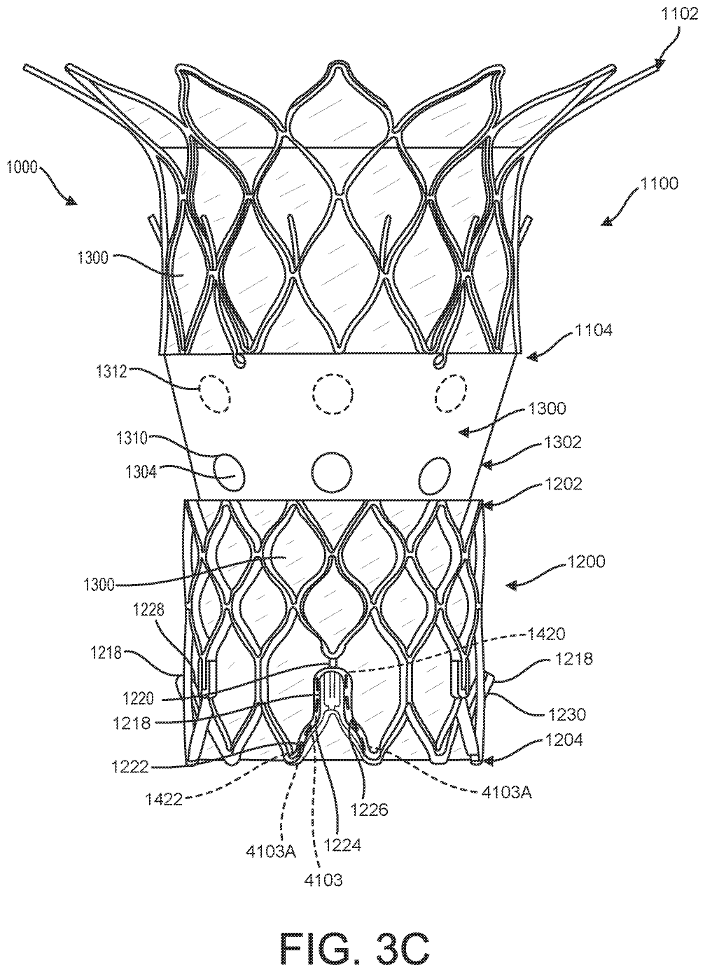

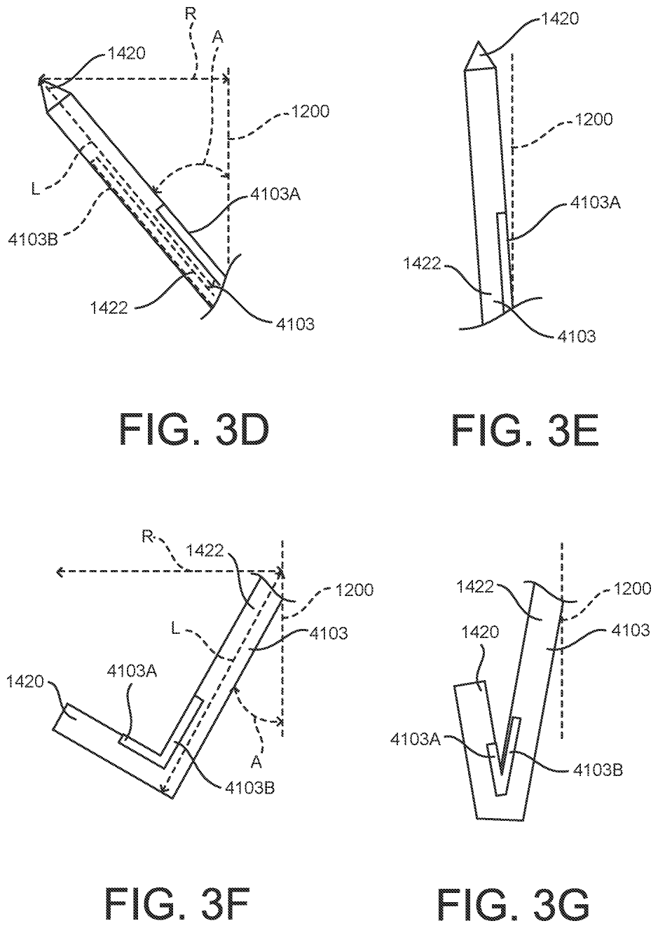

FIG. 3A is a side view of the anchor frame 1101. FIG. 3B is an axial view of the anchor frame subcomponent 1100. The anchor frame subcomponent 1100 includes an anchor frame 1101. The anchor frame 1101 is a tubular member defining an anchor frame lumen 1113 operable to receive the leaflet frame subcomponent 1200 therein. The side of the anchor frame 1101 may be at least partially covered, such as with a film or fabric, not shown for clarity, suitable for a particular purpose, such as, but not limited to, restrict fluid from passing through the anchor frame 1101, or encourage tissue ingrowth at the implant site. For illustrative purposes, the following examples are suitable especially for a transcatheter application, but are also suitable for a surgical application.

In various examples, the construction of and materials used in the film are such that the anchor frame subcomponent 1100 promotes cellular ingrowth, adhesion, and/or attachment. That is, in various examples, the anchor frame subcomponent 1100 is constructed in a manner that promotes the ingrowth of tissue into one or more portions of the film. It is appreciated that cellular ingrowth further increases sealing of the prosthetic valve with the native valve orifice and helps minimize para-valvular leakage, that is, leakage between the prosthetic valve and the tissue into which it is coupled.

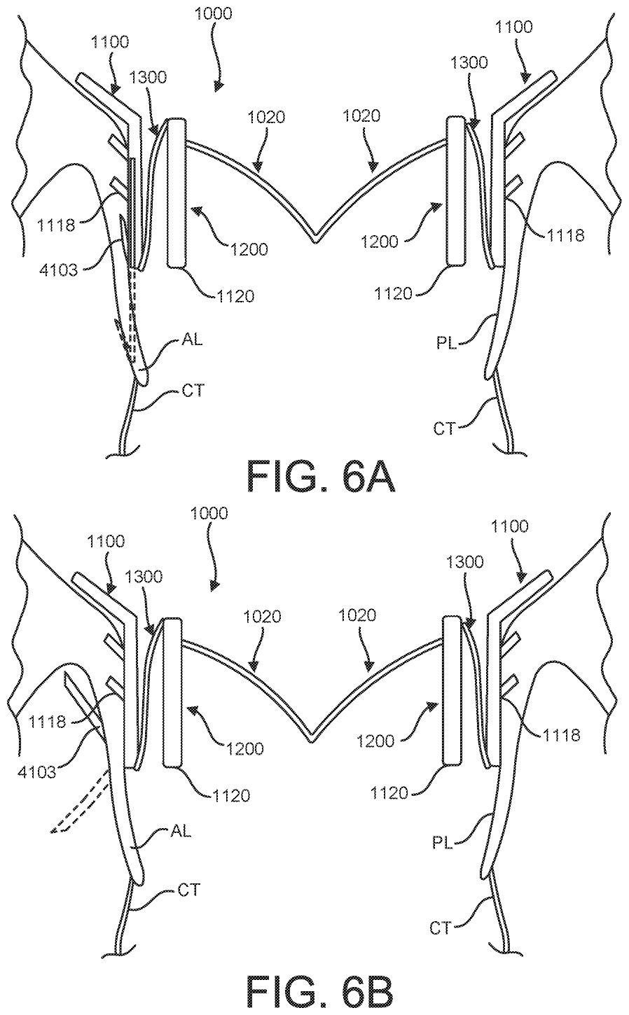

In accordance with some embodiments, the anchor frame subcomponent 1100 and/or the anchor frame 1101 comprise a shape memory material operable to flex under load and retain its original shape when the load is removed, thus allowing the anchor frame subcomponent 1100 to self-expand from a compressed shape to a predetermined larger shape. The anchor frame subcomponent 1100 may comprise the same or different materials as the leaflet frame subcomponent 1200. In accordance with an embodiment, the anchor frame subcomponent 1100 is plastically deformable to be expanded by a balloon. In another embodiment the anchor frame subcomponent 1100 is elastically deformable so as to be self-expanding. Furthermore, the anchor frame subcomponent 1100 may also be coupled to the tissue cutting element 4103, as described below with reference to FIGS. 6A and 6B as well as FIGS. 9A through 9C.

FIGS. 2A and 2B are side and axial views, respectively, of the anchor frame subcomponent 1100, in accordance with an embodiment. The anchor frame subcomponent 1100 is a generally tubular member having an anchor frame subcomponent inlet end 1102, an anchor frame subcomponent outlet end 1104, an anchor frame subcomponent interior surface 1106, and an anchor frame subcomponent exterior surface 1108. In various examples, the anchor frame subcomponent 1100 defines an anchor frame subcomponent interior region 1110 (e.g., an anchor frame subcomponent lumen). For example, the anchor frame subcomponent interior region 1110 is a generally cylindrical void defined between the anchor frame subcomponent inlet end 1102 and the anchor frame subcomponent outlet end 1104, and the anchor frame subcomponent interior surface 1106 of the anchor frame subcomponent 1100. However, in-situ, the anchor frame subcomponent interior region 1110 may adopt an irregular cross section, depending on the geometry of the native valve orifice. In various examples, the anchor frame subcomponent 1100 is configured to couple to a native valve orifice. Accordingly, in various examples, a diameter of the anchor frame subcomponent 1100 (e.g., a diameter of an interior or exterior surface of the anchor frame subcomponent 1100) is sized in accordance with patient anatomy. It will be appreciated that nonlimiting examples of anchor frame subcomponent 1100 can be provided with a diameter (e.g., a diameter of an interior or exterior surface of the anchor frame subcomponent 1100) in a range of between twenty-five (25) millimeters and fifty (50) millimeters, depending on a patient's anatomy. However, examples of the anchor frame subcomponent 1100 having diameters (e.g., a diameter of an interior or exterior surface of the anchor frame subcomponent 1100) in excess of fifty (50) millimeters are also envisioned and fall within the scope of the present disclosure, depending on patient anatomy.

In some embodiments, the anchor frame subcomponent 1100 defines a flange or a flared portion at the anchor frame subcomponent inlet end 1102 that flares or tapers radially outward when in the deployed configuration. For example, as shown in at least FIGS. 1B, 2A, 5B, 5C, and 5E, the anchor frame subcomponent inlet end 1102 is flared or otherwise tapered radially outward when in the deployed configuration. That is, as shown, the anchor frame subcomponent inlet end 1102 of the anchor frame subcomponent 1100 has a larger deployed (e.g. relaxed) diameter than does the anchor frame subcomponent outlet end 1104 of the anchor frame subcomponent 1100. In various examples, as discussed in greater detail below, such a configuration operates to minimize migration risks and helps facilitate abutment of the anchor frame subcomponent 1100 with tissue at the treatment site.

In some embodiments, the anchor frame subcomponent 1100 defines a cylindrical or tubular mesh or crossing-pattern forming a framework defining apertures. For example, as shown, the anchor frame subcomponent 1100 includes a plurality of frame members 1112 that are interconnected and arranged in one or more patterns. In some examples, these patterns repeat one or more times. In some such examples, the frame members 1112 are arranged and interconnected such that the anchor frame subcomponent 1100 includes a plurality of patterned rows. In various examples, the frame members 1112 are connected to one another at joints 1114. In some examples, these joints 1114 operate as flex points so as to provide a preferential flexing location for the anchor frame subcomponent 1100 to flex when compressed to a smaller delivery diameter and when forces from the surrounding anatomy act to compress the anchor frame subcomponent 1100 during normal operation after delivery and deployment of the prosthetic valve 1000. In some examples, the flex point or joints 1114 comprises a site on the anchor frame subcomponent 1100 that undergoes a high degree of bending. In some examples, the joints 1114 may comprise a geometry, structural modification or material modification, among others, that biases the anchor frame subcomponent 1100 to bend at the flex points or joints 1114 when compressed.

In some embodiments, one or more apertures 1116 are defined between the joints 1114 and the frame members 1112 that are interconnected of the anchor frame subcomponent 1100. In some examples, these apertures 1116 extend between the anchor frame subcomponent exterior surface 1108 and the anchor frame subcomponent interior surface 1106 of the anchor frame subcomponent 1100. As illustrated in the embodiments of FIGS. 2A and 2B, one or more of the apertures 1116 define a diamond shape when the anchor frame subcomponent 1100 is in a deployed configuration. Upon compression to a smaller diameter (e.g., a delivery diameter), one or more of the joints 1114 and the frame members 1112 deform such that the apertures 1116 generally define an elongated diamond shape (e.g., as shown generally in FIG. 4). Upon re-expanding the anchor frame subcomponent 1100 to a larger diameter during deployment at a treatment site, the apertures 1116 re-expand to define the generally wider diamond shape.

It should be appreciated that while the frame members 1112 illustrated and described herein are interconnected and define apertures 1116 having generally a diamond shape, the frame members 1112 that are interconnected with one another may be arranged in a number of alternative patterns. For example, a framework of the anchor frame subcomponent 1100 can define any number of features, repeatable or otherwise, such as geometric shapes and/or linear or meandering series of sinusoids. Geometric shapes can comprise any shape that facilitates circumferential compressibility and expandability of the anchor frame subcomponent 1100. That is, a number of alternative patterns are envisioned where the arrangement of frame members 1112 is configured in such a manner as to provide for an anchor frame subcomponent 1100 that can be compressed to a smaller diameter for transcatheter delivery and subsequently expanded (or allowed to expand) to a larger diameter at a treatment site during deployment of the prosthetic valve 1000. Accordingly, the disclosure should not be read as being limited to arrangements of the frame members 1112 that define apertures 1116 that are diamond-shaped.

In various embodiments, the anchor frame subcomponent 1100 may comprise or otherwise be formed from a cut tube, or any other element suitable for the particular purpose of the anchor frame subcomponent 1100 as described herein. In some examples, the anchor frame subcomponent 1100 may be etched, cut, laser cut, or stamped into a tube or a sheet of material, with the sheet then formed into a substantially cylindrical structure. Alternatively, an elongated material, such as a wire, bendable strip, or a series thereof, can be bent or braided and formed into a substantially cylindrical structure wherein the walls of the cylinder comprise an open framework that is compressible to a smaller diameter in a generally uniform and circumferential manner and expandable to a larger diameter as illustrated and described herein.

The anchor frame subcomponent 1100 can comprise any metallic or polymeric biocompatible material. For example, the anchor frame subcomponent 1100 can comprise a material, such as, but not limited to nitinol, cobalt-nickel alloy, stainless steel, or polypropylene, acetyl homopolymer, acetyl copolymer, ePTFE, other alloys or polymers, or any other biocompatible material having adequate physical and mechanical properties to function as described herein.

In various examples, the anchor frame subcomponent 1100 is elastically deformable so as to be self-expanding under spring loads, as those of skill will appreciate. In some examples, the anchor frame subcomponent 1100 is plastically deformable so as to be mechanically expanded such as with a balloon, as those of skill will appreciate. In yet some other examples, the anchor frame subcomponent 1100 is plastically deformable as well as elastically deformable. That is, in some examples, the anchor frame subcomponent 1100 includes one or more elastically deformable components or features and one or more plastically deformable components or features. Thus, it should be appreciated that the examples of the anchor frame subcomponent 1100 presented herein are not to be limited to a specific design or mode of expansion.

### Subcomponent Assembly

In various embodiments, the leaflet frame subcomponent 1200 is nestable within the anchor frame subcomponent interior region 1110 (e.g., the anchor frame lumen 1113) of the anchor frame subcomponent 1100. In particular, as shown, the anchor frame subcomponent 1100 and the leaflet frame subcomponent 1200 are sized and shaped in a manner that provides for the leaflet frame subcomponent 1200 being coaxially disposable or receivable at least partially within the anchor frame subcomponent 1100. Thus, in various examples, the anchor frame subcomponent 1100 is configured such that a portion of (or alternatively all of) the leaflet frame subcomponent 1200 can be received by or otherwise positioned within the anchor frame lumen 1113 defined by the anchor frame subcomponent 1100.

In some examples, the leaflet frame subcomponent 1200 is sized such that a diameter of the exterior surface of the leaflet frame subcomponent 1200 is less than a diameter of the interior surface of the anchor frame subcomponent 1100 that defines the anchor frame lumen 1113. In some examples, a diameter of the exterior surface of the leaflet frame subcomponent 1200 is in a range of between seventy five percent (75%) and ninety percent (90%) of a diameter of the interior surface of the anchor frame subcomponent 1100. In some examples, a diameter of the exterior surface of the leaflet frame subcomponent 1200 is seventy five percent (75%) or less than a diameter of the interior surface of the anchor frame subcomponent 1100. In various examples, such configurations also provide that the leaflet frame subcomponent 1200 can be received within the anchor frame subcomponent 1100. In various examples, such configurations provide that the anchor frame subcomponent 1100 can deform, such as, but not limited to being out of round or generally oval-shaped, to accommodate or otherwise conform to the native valve orifice without causing a deformation of the leaflet frame subcomponent 1200.

Thus, in some examples, the prosthetic valve 1000 provides a leaflet frame subcomponent 1200 that essentially floats within the anchor frame subcomponent 1100 by way of the interstage 1300 and does not directly couple with a native valve orifice. The anchor frame subcomponent 1100 may conform to the shape of the native valve orifice whereas the leaflet frame subcomponent 1200 does not conform to the shape of the native valve orifice. The leaflet frame subcomponent 1200 remains tubular or at a preferred geometrical configuration so as to present the leaflets 1210 with a geometrically stable platform ensuring proper leaflet function, including coaptation and opening dynamics. It is appreciated that these benefits associated with the leaflet frame subcomponent 1200 not needing to conform to the native valve orifice may be realized in either transcatheter or surgical placement of the prosthetic valve 1000.

In various embodiments, the prosthetic valve 1000 is configured such that the anchor frame subcomponent 1100 and the leaflet frame subcomponent 1200 can be nested in-situ after the anchor frame subcomponent 1100 and the leaflet frame subcomponent 1200 are deployed at a treatment site in a patient's anatomy. That is, in various embodiments, the prosthetic valve 1000 can be delivered to a treatment region within a patient's anatomy with the anchor frame subcomponent 1100 and the leaflet frame subcomponent 1200 longitudinally offset relative to one another and subsequently nested with one another at the treatment site. In various embodiments, the prosthetic valve 1000 is loaded onto a delivery device with the anchor frame subcomponent 1100 and the leaflet frame subcomponent 1200 longitudinally offset relative to one another which presents a lower profile or diameter than if the prosthetic valve 1000 were to be loaded onto the delivery device in the nested configuration. A lower delivery profile of a transcatheter delivered prosthetic valve has well recognized advantages, including easier advancement though vessels.

It is appreciated that these benefits associated with the leaflet frame subcomponent 1200 not being nested into the anchor frame subcomponent 1100 during implantation may also be realized in surgical placement of the prosthetic valve 1000. By way of example, but not limited thereto, the anchor frame subcomponent 1100 may be more easily sutured into the native valve orifice without the leaflet frame subcomponent 1200 being within the anchor frame subcomponent 1100 and in close proximity to the suturing procedure lessening the chance of needle damage to the leaflets.

In some embodiments, the anchor frame subcomponent 1100 and the leaflet frame subcomponent 1200 are operable to nest with one another by telescoping the anchor frame subcomponent 1100 and the leaflet frame subcomponent 1200 relative to one another in-situ. Thus, in various examples, the leaflet frame subcomponent 1200 and the anchor frame subcomponent 1100 are sized such that the leaflet frame subcomponent 1200 can be receive within an anchor frame subcomponent interior region 1110.

In various embodiments, in addition to or alternative to telescoping relative to one another, the anchor frame subcomponent 1100, the leaflet frame subcomponent 1200, and the interstage 1300 are each configured to be compressed or collapsed to a delivery profile and then re-expanded in-situ to provide for transcatheter delivery of the prosthetic valve 1000, as discussed in greater detail below.

Tissue Engagement Features

In various embodiments, the anchor frame subcomponent 1100 is configured to provide positive engagement with an implant site to firmly anchor the prosthetic valve 1000 to the treatment site. For instance, in various examples, the anchor frame subcomponent 1100 includes one or more tissue engagement features 1118 that are configured to engage one or more regions of tissue at the native valve orifice adjacent the prosthetic valve 1000. In various examples, the tissue engagement features 1118 comprise one or more tissue anchors (e.g., barbs). In various examples, the length of the tissue engagement features 1118 is shorter than the length of the tissue cutting element 4103.

In various examples, the one or more tissue engagement features 1118 project away from the anchor frame subcomponent interior and/or exterior surfaces 1106 and 1108 of the anchor frame subcomponent 1100, radially outward from a longitudinal axis of the anchor frame subcomponent 1100, and toward the tissue adjacent to the prosthetic valve 1000. Generally, the tissue engagement features 1118 may be operable to project away from the anchor frame subcomponent 1100 when the anchor frame subcomponent 1100 is deployed (e.g., when a constraining member is withdrawn or otherwise removed). Although, in some examples, the tissue engagement features 1118 may be independently deployable. In some examples, with the anchor frame subcomponent 1100 in the deployed configuration, the tissue engagement features 1118 are operable to engage the tissue proximate the anchor frame subcomponent 1100 such that the tissue engagement features 1118 secure the anchor frame subcomponent 1100 to the adjacent tissue, as will be discussed in greater detail below.

In some examples, in a deployed configuration, the tissue engagement features project away from an exterior surface of the anchor frame subcomponent in a range of between thirty (30) and sixty (60) degrees. In some such examples, the tissue engagement features project away from an exterior surface, or offset from the exterior surface, of the anchor frame subcomponent at an angle of forty-five (45) degrees, though other configurations are contemplated and fall within the scope of the present application, including any approximate value of the foregoing values. Generally, any angle of projection is suitable provided that the tissue engagement features operate for their intended purpose of engaging the tissue adjacent to the anchor frame subcomponent and causing the anchor frame subcomponent to be secured to the tissue. Though the tissue engagement features may include a variety of different lengths (depending on the angle from which they project from the anchor frame subcomponent), it will be appreciated that the tissue engagement features are of a length suitable for engaging tissue and securing the anchor frame subcomponent to the adjacent tissue, but not so long as to risk detrimental damage to the native valve orifice. One nonlimiting example configuration includes tissue engagement features projecting from the anchor frame subcomponent in a range of between thirty (30) and sixty (60) degrees and having a length of between fifty (50) micron and two hundred (200) micron.

Generally, the tissue engagement features 1118 are positioned along the anchor frame subcomponent such that they are operable to engage tissue proximate the anchor frame subcomponent 1100 when the anchor frame subcomponent 1100 is expanded in-situ. The tissue engagement features 1118 may be arranged in one or more rows along a longitudinal axis of the anchor frame subcomponent 1100. That is, in various examples, anchor frame subcomponent may include a first set (or row) of anchors and a second set (or row) of anchors longitudinally offset relative to the first set of anchors. In one such example, the first set of anchors is more proximate the anchor frame subcomponent outlet end 1104 of the anchor frame subcomponent 1100 than is the second set of anchors.

In various embodiments, the one or more tissue engagement features 1118 are circumferentially arranged about the anchor frame subcomponent 1100. In some examples, the one or more tissue engagement features 1118 are evenly dispersed about the circumference of the anchor frame subcomponent. For example, the tissue engagement features 1118 are dispersed about the frame and are offset from one another by ninety (90) degrees depending on the number of anchors. Alternatively, the tissue engagement features 1118 may be dispersed about the frame and offset from one another by sixty (60) degrees depending on the number of anchors. Generally, the angular offset between the anchors is a function of the number of anchors dispersed about the anchor frame subcomponent 1100, as those of skill will appreciate. In some examples, the angular offset between the anchors is additionally or alternatively based on an arrangement or pattern of the frame members 1112.

In various examples, while the tissue engagement features 1118 project away from the anchor frame subcomponent 1100 when the anchor frame subcomponent 1100 is in the deployed configuration, the tissue engagement features 1118 are stowed or do not otherwise project away from the anchor frame subcomponent 1100 when the anchor frame subcomponent 1100 is compressed in the delivery configuration. Thus, in various examples, the tissue engagement features 1118 are stowable during delivery and are configured to transition to a deployed configuration where they project away from the anchor frame subcomponent 1100. In some examples, a constraining member disposed about the anchor frame subcomponent 1100 during delivery facilitates stowing of the tissue engagement features 1118. In some examples, the tissue engagement features 1118 are stowed in one or more of the apertures 1116 of the anchor frame subcomponent 1100.

In various embodiments, the tissue engagement features 1118 are integral to the anchor frame subcomponent 1100. For example, one or more of the tissue engagement features 1118 are formed in conjunction with and from the same material as the frame members 1112. In other examples, one or more of the tissue engagement features 1118 are separate components additionally or alternatively coupled or attached to the anchor frame subcomponent 1100. For instance, some non-limiting examples include crimping and/or welding one or more tissue engagement features to the anchor frame subcomponent 1100.

Likewise, while the anchor frame subcomponent inlet end 1102 of the anchor frame subcomponent 1100 tapers or flares radially outward in a deployed configuration in certain examples, the flared or tapered portion of the anchor frame subcomponent 1100 is configured to deflect when the anchor frame subcomponent 1100 is in the delivery configuration. For example, as shown in FIG. 4, the anchor frame subcomponent inlet end 1102 of the anchor frame subcomponent 1100 is deflected such that the anchor frame subcomponent 1100 has a substantially uniform delivery profile along its longitudinal axis.

In various examples, one or more constraining members (not shown) are formed as a filament or other material that can be looped or extended around various features and placed under tension to maintain those features in a compact, or delivery configuration. In some example, the one or more constraining members generally define a loop and are disposed about the anchor frame subcomponent 1100 in the delivery configuration. For example, a first constraining member is disposed near the anchor frame subcomponent inlet end 1102 of the anchor frame subcomponent 1100 and a second constraining member is disposed near the anchor frame subcomponent outlet end 1104 of the anchor frame subcomponent 1100. Each constraining member may extend about an anchor frame subcomponent exterior surface 1108 of the anchor frame subcomponent 1100, or one or more of the constraining members may be woven through one or more portions of the film disposed about the anchor frame subcomponent 1100. That is, in some examples, one or more of the constraining members extending adjacent the anchor frame subcomponent exterior surface 1108 (e.g., exterior of the anchor frame subcomponent exterior surface) may extend through a portion of the film, and extend along a portion of the anchor frame subcomponent interior surface 1106 or adjacent to the anchor frame subcomponent interior surface (e.g., interior of the anchor frame subcomponent interior surface) of the anchor frame subcomponent 1100, and then extend back through the film to a location exterior or against the anchor frame subcomponent exterior surface 1108 and extend therearound. In some examples, the one or more constraining members individually or collectively operate to constrain the anchor frame subcomponent 1100 in a delivery configuration. In various examples, this includes one or more constraining members individually or collectively constrains the flange or flared portion of the anchor frame subcomponent 1100 in a delivery configuration. Additionally or alternatively, in some examples, a removable constraining sheath is disposed about the flange or flared portion of the anchor frame subcomponent 1100 in a delivery configuration. In some examples, one or more constraining members individually or collectively constrain the tissue engagement features to a delivery (undeployed) configuration. Additionally or alternatively, in some examples, a removable constraining sheath is disposed about the tissue engagement features of the anchor frame subcomponent 1100. In some examples, the one or more constraining members are removed from the anchor frame subcomponent 1100 during deployment of the anchor frame subcomponent 1100. In some examples, the constraining members includes a fiber. In some examples, the constraining members includes a wire. In some examples, one or more lockwires engage a first end of the one or more constraining members at or proximate the anchor frame subcomponent 1100 such that tension can be applied to an opposing second end of the one or more constraining members. In various examples, tensioning the one or more constraining members operates to maintain the anchor frame subcomponent 1100 in the delivery configuration.

In various examples, one or more constraining members are disposed about the leaflet frame subcomponent 1200 in the delivery configuration. For example, a third constraining member is disposed about the leaflet frame subcomponent inlet end 1202 of the leaflet frame subcomponent 1200 and a fourth constraining member is disposed about the leaflet frame subcomponent outlet end 1204 of the leaflet frame subcomponent 1200. Each constraining member may extend about a leaflet frame subcomponent exterior surface 1208 of the leaflet frame subcomponent 1200. In some such examples, one or more of the constraining members may be woven through one or more portions of the film disposed about the leaflet frame subcomponent 1200. In some examples, one or more of the constraining members that extend circumferentially about the leaflet frame subcomponent exterior surface 1208, or otherwise extend through a portion of the film, and extend along a portion of the leaflet frame subcomponent interior surface 1206 of the leaflet frame subcomponent 1200, and then extend back through the film to the leaflet frame subcomponent exterior surface 1208 and extend therearound. In some examples, the one or more constraining members individually or collectively operate to constrain the leaflet frame subcomponent 1200 in a delivery configuration. In various examples, one or more constraining members individually or collectively constrain the tissue cutting element to a delivery (undeployed) configuration. Additionally or alternatively, in some examples, a removable constraining sheath is disposed about the tissue engagement features of the leaflet frame subcomponent 1200. It is appreciated that the removable constraining sheath may be disposed about both the leaflet frame subcomponent 1200 and the anchor frame subcomponent 1100 (see discussion above). In some examples, the one or more constraining members are removed from the leaflet frame subcomponent 1200 during deployment of the leaflet frame subcomponent 1200. In some examples, the constraining members includes a fiber. In some examples, the constraining members includes a wire. In some examples, one or more lockwires engage a first end of the one or more constraining members at or proximate the leaflet frame subcomponent 1200 such that tension can be applied to an opposing second end of the one or more constraining members. In various examples, tensioning the one or more constraining members operates to maintain the leaflet frame subcomponent 1200 in the delivery configuration.

### Interlock Features

In various embodiments, in addition to facilitating a positive engagement with an implant site to anchor the prosthetic valve 1000 to the surrounding tissue, the anchor frame subcomponent 1100 additionally or alternatively includes one or more mechanisms that facilitate a positive engagement with the leaflet frame subcomponent 1200 upon nesting the anchor frame subcomponent 1100 and the leaflet frame subcomponent 1200. Specifically, in various examples, the anchor frame subcomponent 1100 includes one or more interlock features 1120 that project into the anchor frame subcomponent interior region 1110 of the anchor frame subcomponent 1100. These interlock features 1120 are configured to engage the leaflet frame subcomponent 1200 as nested and maintain a relative axial position (or at least minimize relative axial movement) between the anchor frame subcomponent 1100 and the leaflet frame subcomponent 1200. Thus, according to various examples, one or more interlock features 1120 are incorporated with the prosthetic valve 1000 and operate to help maintain a coupling between the leaflet frame subcomponent 1200 and the anchor frame subcomponent 1100.

In various examples, the interlock features 1120 are structures that project or otherwise extend away from the anchor frame subcomponent interior and/or exterior surfaces 1106 and 1108, respectively, of the anchor frame subcomponent 1100 and toward the anchor frame subcomponent interior region 1110 defined by the anchor frame subcomponent 1100. In some examples, the one or more interlock features 1120 are in the form of one or more tabs.

In some examples, the one or more interlock features 1120 have a free end 1122 and a base 1124. In some examples, the free end 1122 is an end that is not otherwise coupled to or mated with the anchor frame subcomponent 1100. The base 1124 is generally the portion of the interlock feature that couples to or is otherwise integral with the anchor frame subcomponent 1100. Generally, the free end 1122 is operable to move relative to the anchor frame subcomponent 1100, while the base 1124 is coupled to the anchor frame subcomponent 1100.

Though a variety of geometries are envisioned, the non-limiting examples of the interlock features 1120 illustrated in FIGS. 2A and 2B are each elongate elements. In addition, the free end 1122 is illustrated as being a generally blunt or round end, though the free end 1122 of the one or more interlock features 1120, generally, may alternatively be pointed or possess other suitable geometry such as a curved shape (e.g., an s-shape). In other words, other geometries suitable for engaging the leaflet frame subcomponent 1200 when it is nested with the anchor frame subcomponent 1100 in the manner illustrated and described herein are envisioned and may be utilized without departing from the spirit or scope of the disclosure. In some examples, the free end 1122 of the one or more interlock features 1120 is shaped such that it is operable to slide along the exterior of the leaflet frame subcomponent 1200. As mentioned above, in some examples, a film or other covering material covers one or more portions of the leaflet frame subcomponent 1200. Thus, in some examples, the free end 1122 of the one or more interlock features 1120 is shaped and sized in a manner that allows the one or more interlock features 1120 to slide along the exterior of the leaflet frame subcomponent 1200 without binding. In one nonlimiting example, the one or more interlock features 1120 are from five hundred (500) microns in length to two (2) millimeters in length (e.g., six-hundred (600) microns) and are angled from fifteen (15) degrees to seventy-five degrees (e.g., forty-five (45) degrees) relative to longitudinal axis of the anchor frame subcomponent 1100 and/or the interior of the anchor frame subcomponent 1100. It will be appreciated, however, that a number of angle and length configurations are contemplated and fall within the scope of the present application, including approximate values of any of the foregoing.

Similar to the tissue engagement features 1118, the interlock features 1120 may be arranged in one or more rows along a longitudinal axis of the anchor frame subcomponent 1100. That is, in various examples, anchor frame subcomponent 1100 may include a first set (e.g., a row) of interlock features and a second set (e.g., a row) of interlock features longitudinally offset relative to the first set of interlock features. In one such example, the first set of interlock features is more proximate the anchor frame subcomponent outlet end 1104 of the anchor frame subcomponent 1100 than is the second set of interlock features. In various examples, while the interlock features 1120 are configured to project away from the anchor frame subcomponent 1100 when the anchor frame subcomponent 1100 is in the deployed configuration, the interlock features 1120 are stowed or do not otherwise project away from the anchor frame subcomponent 1100 when the anchor frame subcomponent 1100 is compressed in the delivery configuration. Thus, in various examples, the interlock features 1120 are configured to transition between a stowed or delivery configuration and a projecting or deployed configuration. Thus, in various examples, the interlock features 1120 are resilient members that are configured to deflect under certain conditions.

In various examples, as mentioned above, the interlock features 1120 are configured to engage the leaflet frame subcomponent 1200 as it is nested with the anchor frame subcomponent 1100 in-situ. In some examples, as discussed further below, the interlock features 1120 temporarily deflect from an engaged position to facilitate nesting of the leaflet frame subcomponent 1200 with the anchor frame subcomponent 1100, and subsequently return to the engaged position after the leaflet frame subcomponent 1200 is nested with the anchor frame subcomponent 1100. In various examples, the interlock features 1120 return to the engaged position upon the leaflet frame subcomponent 1200 being proximally advanced a suitable amount relative to the anchor frame subcomponent 1100. Put differently, in some examples, the interlock features 1120 of the anchor frame subcomponent 1100 are operable to adopt an engaged position where they engage the leaflet frame subcomponent 1200 and minimize relative axial translation between the leaflet frame subcomponent 1200 and the anchor frame subcomponent 1100 upon proximally advancing the leaflet frame subcomponent 1200 a designated amount relative to the anchor frame subcomponent 1100.

In some examples, a delivery device upon which the anchor frame subcomponent 1100 is loaded during delivery causes stowing of the interlock features 1120.

In various examples, the interlock features 1120 are integral to the anchor frame subcomponent 1100. For example, one or more of the interlock features 1120 are formed in conjunction with and from the same material as the frame members 1112. In other examples, one or more of the interlock features 1120 are additionally or alternatively coupled to the anchor frame subcomponent 1100. That is, in some examples, one or more interlock features 1120 are additionally or alternatively attached to the anchor frame subcomponent 1100. In various examples, the one or more interlock features 1120 are circumferentially arranged about the anchor frame subcomponent 1100. In some examples, the one or more interlock features 1120 are evenly dispersed about the circumference of the anchor frame subcomponent. In a manner similar to that discussed above with respect to the tissue engagement features 1118, the angular offset between the anchors is generally a function of one or more of the arrangement of the frame members 1112 and the number of anchors dispersed about the anchor frame subcomponent 1100, as those of skill will appreciate.

It should be appreciated that while the interlock features are illustrated and described herein as extending from the anchor frame subcomponent 1100, in various examples, one or more interlock features additionally or alternatively extend from the leaflet frame subcomponent 1200. For instance, in some examples, the leaflet frame subcomponent includes one or more interlock features (not shown) that extend from the leaflet frame subcomponent exterior surface 1208 away from the leaflet frame subcomponent interior surface 1206 and that are operable to engage the anchor frame subcomponent 1100 upon nesting of the anchor frame subcomponent 1100 and the leaflet frame subcomponent 1200. In various examples, the interlock features of the leaflet frame subcomponent 1200 are positionable at a leaflet frame subcomponent inlet end 1202, a leaflet frame subcomponent outlet end 1204, or some position between the leaflet frame subcomponent inlet and outlet ends 1202 and 1204 provided that the interlock features of the leaflet frame subcomponent 1200 are operable to engage the anchor frame subcomponent 1100 upon nesting of the anchor frame subcomponent 1100 and the leaflet frame subcomponent 1200. In various examples, the interlock features of the leaflet frame subcomponent are deflectable and stowable in a manner similar to the interlock features 1120 of the anchor frame subcomponent 1100, as previously described.

FIGS. 3A and 3B are side and axial views, respectively, of the leaflet frame subcomponent 1200, in accordance with an embodiment. The leaflet frame subcomponent 1200 is generally cylindrical or tubular member having a leaflet frame subcomponent inlet end 1202, a leaflet frame subcomponent outlet end 1204, a leaflet frame subcomponent interior surface 1206, and a leaflet frame subcomponent exterior surface 1208. In various examples, the leaflet frame subcomponent 1200 defines a leaflet frame subcomponent interior region 1209. For example, leaflet frame subcomponent interior region 1209 is a generally cylindrical void defined between the leaflet frame subcomponent inlet and outlet ends 1202 and 1204, and the leaflet frame subcomponent interior surface 1206 of the leaflet frame subcomponent 1200. Generally, the leaflet frame subcomponent 1200 is configured to be received within at least a portion of the anchor frame subcomponent 1100, as mentioned above. It will be appreciated that nonlimiting examples of the leaflet frame subcomponent 1200 can be provided with a diameter (e.g., a diameter of an interior or exterior surface of the leaflet frame subcomponent 1200) in a range of between twenty (20) millimeters and thirty (30) millimeters, depending on a patient's anatomy.

Tissue Retention Features

As shown in FIG. 3C, in various examples, the prosthetic valve 1000 (e.g., the leaflet frame subcomponent 1200) optionally includes one or more features that operate to grab or otherwise interface with native valve tissue (e.g., native leaflet tissue) or tissue surrounding the native valve being replaced. For example, the leaflet frame subcomponent 1200 (or another portion of the prosthetic valve 1000, such as the anchor frame subcomponent 1100) optionally includes one or more tissue retention features 1218 (also referred to herein as tissue graspers). The one or more tissue retention features 1218 may be formed as projections (e.g., projections of the leaflet frame subcomponent 1200) that are configured to interface with the patient's native tissue associated with the native valve.

In some examples, the one or more tissue retention features 1218 are configured to engage the native tissue. In some examples, the tissue retention features 1218 are configured to cause native tissue to be secured between the anchor frame subcomponent 1100 and the leaflet frame subcomponent 1200 as the anchor frame subcomponent 1100 and the leaflet frame subcomponent are nested together in-situ.

In some examples, the tissue retention features 1218 are configured to retain or secure native tissue without securing the native tissue between the leaflet frame subcomponent 1200 and the anchor frame subcomponent 1100. For example, the tissue retention features 1218 may project radially outward beyond the anchor frame subcomponent 1100. In such instances, the tissue retention features 1218 may be passed beyond the free edge of the tissue to be manipulated (e.g., the anterior leaflet (AL)), and then retracted to retain or secure the tissue. In still further examples, the anchor frame subcomponent 1100 may not be present, and in such instances the tissue retention features 1218 engage and retain or secure native tissue without regard to the anchor frame subcomponent 1100. Such configurations can help avoid the native tissue interfering with or otherwise obstructing the flow of fluid (e.g., blood) downstream or antegrade to the prosthetic valve 1000 after the prosthetic valve 1000 has been deployed.

In mitral valve repair/augmentation procedures for example, the capture and securement of at least the native anterior leaflet of the native mitral valve minimized that potential for the native anterior leaflet to deflect into the left ventricle and create a left ventricle outflow tract obstruction. Thus, in various embodiments, the one or more tissue retention features 1218 are configured to interface with one or more of the native leaflets associated with the native valve. Though mitral valve repair/augmentation procedures are discussed herein, it will be appreciated that the scope of the disclosure applies to repair/augmentation of the atrioventricular (AV) valves and the semilunar (SL) valves. The disclosure should therefore not be interpreted as being limited to mitral valve repair/augmentation.

In various examples, the tissue retention features 1218 are structures that project or otherwise extend away from the leaflet frame subcomponent 1200 and toward the tissue surrounding the prosthetic valve 1000 (e.g., the native valve orifice). In some examples, the one or more tissue retention features 1218 are in the form of one or more tabs. In some examples, the one or more tissue retention features 1218 are looped features having an apex and two ends, wherein the two ends are coupled to, integral with, extend from, or otherwise terminate into one or more portions of the leaflet frame subcomponent 1200. In some such examples, the apex is a free end that is operable to deflect and project away from the leaflet frame subcomponent 1200, as mentioned below.

In some examples, the one or more tissue retention features 1218 have a free end 1220 and a base 1222. In some examples, the free end 1220 is an end that is not otherwise coupled to or mated with the leaflet frame subcomponent 1200. The base 1222 includes one or more portions of the one or more tissue retention features 1218 that couple to or are otherwise integral with the leaflet frame subcomponent 1200. Generally, the free end 1220 is operable to move relative to the leaflet frame subcomponent 1200, while the base 1222 is coupled to the leaflet frame subcomponent 1200.

In some examples, the tissue retention features are simple barbs or hook features formed similarly to anchoring barbs, but in an inverted configuration. Though a variety of geometries are envisioned, another non-limiting exemplary design for the tissue retention features 1218 is illustrated in FIG. 3C as a generally triangularly shaped feature with a blunt end (though sharp ends and other features are contemplated for the tissue retention features 1218, as described below). As shown, each of the tissue retention features 1218 include a free end 1220 and a base 1222. Each tissue retention feature includes a first leg 1224 and a second leg 1226 that are each coupled to, integral with, extend from, or otherwise terminate into the leaflet frame subcomponent 1200.

As shown, the first leg 1224 and the second leg 1226 converge to form the free end 1220. In addition, while the free end 1220 is illustrated as being a generally blunt or round end, the free end 1220 may alternatively be pointed or possess other suitable geometry. In other words, other geometries suitable for engaging surrounding tissue in the manner illustrated and described herein are envisioned and may be utilized without departing from the spirit or scope of the disclosure. For instance, another non-limiting exemplary tissue retention feature includes an end coupled to or otherwise integral with the leaflet frame subcomponent 1200 and a plurality of free ends extending from the end coupled to the leaflet frame subcomponent 1200. Another non-limiting exemplary tissue retention feature includes a barb or similar feature having opposed single ends coupled to or otherwise integral with the leaflet frame subcomponent 1200. The profile of the free end 1220 of the tissue retention feature 1218 may be one well suited for penetrating tissue (e.g., piercing) or penetrating between tissue (e.g., hooking) of the surrounding anatomy.

In various examples, the tissue retention features 1218 have a first side 1228 and a second side 1230. As shown, the first side 1228 faces the leaflet frame subcomponent exterior surface 1208 of the leaflet frame subcomponent 1200, and the second side 1230 faces away from the leaflet frame subcomponent exterior surface 1208 of the leaflet frame subcomponent. In some examples, a void or open space region is defined between the first side 1228 and the leaflet frame subcomponent exterior surface 1208 of the leaflet frame subcomponent 1200. In various examples, this open space region between the first side 1228 and the leaflet frame subcomponent exterior surface 1208 of the leaflet frame subcomponent 1200 is configured to accommodate a portion of native tissue (e.g., valve leaflets) from anatomy surrounding the prosthetic valve 1000. As previously referenced, the tissue retention features 1218 may also be configured to retain or secure native tissue without securing the native tissue between the leaflet frame subcomponent 1200 and the anchor frame subcomponent 1100. Again, the tissue retention features 1218 may project radially outward beyond the anchor frame subcomponent 1100 or, the anchor frame subcomponent 1100 may not be present according to various examples, and in such instances the tissue retention features 1218 engage and retain or secure native tissue without regard to the anchor frame subcomponent 1100.

Generally, the one or more tissue retention features 1218 of the leaflet frame subcomponent 1200 are situated along the leaflet frame subcomponent 1200 proximate the leaflet frame subcomponent outlet end 1204. In some examples, the base 1222 of the one or more tissue retention features 1218 forms part of the distal end of the leaflet frame subcomponent 1200. In other examples, the base 1222 of the one or more tissue retention features 1218 is situated proximal to the leaflet frame subcomponent outlet end 1204 of the leaflet frame subcomponent 1200. Thus, the one or more tissue retention features 1218 can be generally located at any position along the longitudinal axis of the leaflet frame subcomponent 1200 provided that the tissue retention features 1218 are appropriately sized and shaped for causing native tissue to be captured (e.g., between the anchor frame subcomponent 1100 and the leaflet frame subcomponent 1200), such as upon nesting of the anchor frame subcomponent 1100 where present and the leaflet frame subcomponent 1200, or as otherwise described.

In various examples, the one or more tissue retention features 1218 are circumferentially arranged about the leaflet frame subcomponent 1200. In some examples, the one or more tissue retention features 1218 are evenly dispersed about the circumference of the anchor frame subcomponent. For example, the tissue retention features 1218 are dispersed about the frame and are offset from one another by ninety (90) degrees depending on the number of tissue retention features. Alternatively, the tissue retention features 1218 may be dispersed about the frame and offset from one another by sixty (60) degrees, or some other angular offset, depending on the number of tissue retention features. Generally, the angular offset between the anchors is a function of the number of tissue retention features dispersed about the leaflet frame subcomponent 1200, as those of skill will appreciate. In some examples, the angular offset between the tissue retention features is additionally or alternatively based on an arrangement or pattern of the frame members 1212. Such configurations provide for a prosthetic valve that is deployable in virtually any angular orientation about the longitudinal axis of the prosthetic valve 1000. That is, such configurations minimize the need for physicians to orient the prosthetic valve 1000 about a longitudinal axis of the prosthetic valve 1000 relative to the surrounding native tissue.

In some examples, the tissue retention features 1218 are dispersed about the leaflet frame subcomponent 1200 based on the anatomy of the native tissue surrounding the natural valve to be replaced by the prosthetic valve. For example, the mitral valve is comprised of two native leaflets. In exemplary embodiments including a prosthetic valve configured for implantation to repair or augment a damaged or faulty native mitral valve, the tissue retention features of the leaflet frame subcomponent may be more heavily distributed within certain angular regions to increase the number of tissue retention features in proximity to the native leaflets to capture the native leaflets.

In various examples, as mentioned above, the one or more tissue retention features 1218 project away from the leaflet frame subcomponent 1200 toward the surrounding tissue when the leaflet frame subcomponent 1200 is in the deployed configuration. In some examples, the one or more tissue retention features 1218 project away from the leaflet frame subcomponent 1200 such that the free end 1220 of the tissue retention feature 1218 is more radially offset from an axis of the leaflet frame subcomponent 1200 (e.g., extends more radially outwardly) than is the base 1222 of the tissue retention feature 1218. In other words, in various examples, one or more of the tissue retention feature 1218 are angled relative to a longitudinal axis of the leaflet frame subcomponent 1200 and/or the leaflet frame subcomponent exterior surface 1208 of the leaflet frame subcomponent 1200 when the leaflet frame subcomponent 1200 is in the deployed configuration. Such a configuration provides that the open space region defined between the first side 1228 and the leaflet frame subcomponent exterior surface 1208 of the leaflet frame subcomponent 1200 is tapered. In some examples, the open space region is wedge-shaped.

In various examples, a length and angle configuration of the tissue retention features 1218 is based on the relative sizes of the anchor frame subcomponent 1100 and the leaflet frame subcomponent 1200. For example, the length and angle configuration of the tissue retention features 1218 is such that the tissue retention features 1218 do not prevent or otherwise obstruct the leaflet frame subcomponent 1200 from telescoping or otherwise being nested with the anchor frame subcomponent 1100. Additionally, however, the length and angle configuration of the tissue retention features 1218 is one that provides for the tissue engagement features engaging one or more of the native leaflets of the patient's anatomy, as discussed herein. In some nonlimiting examples, the tissue retention features 1218 have a length of between five-hundred (500) micrometers and 20 mm and project away from the leaflet frame subcomponent 1200 at an angle in a range of between thirty (30) and sixty (60) degrees. Accordingly, though a variety of other configurations are contemplated, one nonlimiting example configuration includes tissue engagement features having a length of approximately five (5) to ten (10) millimeters that project away from the leaflet frame subcomponent 1200 in the deployed configuration at an angle of approximately forty-five (45) degrees.

In various examples, the tissue retention feature 1218 is angled between fifteen (15) and forty five (45) degrees relative to the longitudinal axis of the leaflet frame subcomponent 1200. For instance, in some examples, when deployed, the tissue retention feature 1218 of the leaflet frame subcomponent 1200 is angled at approximately thirty (30) degrees relative to a longitudinal axis of the leaflet frame subcomponent 1200. Generally the tissue retention feature 1218 may be angled less than fifteen (15) or alternatively more than forty five (45) degrees relative to the longitudinal axis of the leaflet frame subcomponent 1200, though as the angle approaches zero (0) degrees and ninety (90) degrees, the ability of the tissue retention feature 1218 to engage and capture tissue diminishes.

In various examples, the tissue retention features 1218 of the leaflet frame subcomponent 1200 are generally oriented such that the free ends 1220 are situated proximal to the bases 1222 of the tissue retention features 1218. As discussed in greater detail below, such a configuration provides for a tissue retention feature that is operable to engage and capture native tissue (e.g., as the leaflet frame subcomponent 1200 and the anchor frame subcomponent 1100 are nested in-situ) and may cause the native tissue to be captured between the leaflet frame subcomponent 1200 and anchor frame subcomponent 1100.

In various examples, while the tissue retention features 1218 are configured to project away from the leaflet frame subcomponent 1200 when the leaflet frame subcomponent 1200 is in the deployed configuration, the tissue retention features 1218 are stowed or do not otherwise project away from the leaflet frame subcomponent 1200 when the leaflet frame subcomponent 1200 is compressed or collapsed in the delivery configuration. In some examples, a constraining member disposed about the leaflet frame subcomponent 1200 during delivery cases stowing of the tissue retention features 1218. In some examples, the tissue retention features 1218 are stowed in apertures 1216 of the leaflet frame subcomponent 1200. Thus, in various examples, the tissue retention features 1218 are configured to transition between a stowed or delivery configuration and a projecting or deployed configuration.

In some examples, the tissue retention features 1218 are resilient structures. In some examples, the tissue retention features 1218 are biased to project away from the leaflet frame subcomponent 1200. In other words, in various examples the tissue retention features 1218 naturally project away from the leaflet frame subcomponent 1200 upon the leaflet frame subcomponent 1200 expanding to the deployed configuration (or the constraining member otherwise being removed).

In various examples, the tissue retention features 1218 are integral to the leaflet frame subcomponent 1200. For example, one or more of the tissue retention features 1218 are formed in conjunction with and from the same material as the frame members 1212. In other examples, one or more of the tissue retention features 1218 are additionally or alternatively coupled to the anchor frame subcomponent 1100.

### Tissue Cutting Elements

In various examples, the prosthetic valve 1000 includes one or more tissue cutting elements 4103. As shown in FIGS. 1A, 2A, and 3C to 3G, a variety of different tissue cutting element configurations are contemplated. In various examples, the tissue cutting element 4103 extends from a base 1422 to a free end 1420 (e.g., in a manner of a cantilever) that is operable to deflect and project away from the prosthetic valve 1000 (e.g., the leaflet frame subcomponent 1200). In some examples, the tissue cutting element 4103 is an elongated element extending in a single direction between the free end 1420 and the base 1422 (e.g., as shown in FIGS. 1A, 3D, and 3E). In other examples, the tissue cutting element includes one or more bends, curves, or angled sections to define more complex shapes (e.g., as shown in FIGS. 3C, 3F, and 3G).

In various examples, the tissue cutting element 4103 is a structure that is capable of moving to an active position or active state (e.g., FIGS. 1A, 3C, 3D, and 3F) in which the tissue cutting element 4103 projects or otherwise extends away from the leaflet frame subcomponent exterior surface 1208 of the leaflet frame subcomponent 1200 and toward tissue adjacent to the prosthetic valve 1000 (e.g., the valve orifice).

In various examples, the base 1422 of each tissue cutting element 1403 includes a portion of the tissue cutting element 4103 that is coupled to or is integral with the prosthetic valve 1000 (e.g., as an integral part of the leaflet frame subcomponent 1200, and more specifically, with the leaflet frame 1201). Generally, the free end 1420 is operable to move relative to the prosthetic valve 1000 (e.g., the leaflet frame subcomponent 1200), while the base 1422 is coupled to the prosthetic valve 1000 (e.g., the leaflet frame subcomponent 1200). In some examples, the one or more tissue cutting elements 4103 may be coupled to the leaflet frame subcomponent 1200 and/or other portions of the prosthetic valve 1000 (e.g., the anchor frame subcomponent 1100) as desired. The tissue cutting elements 4103 may be configured to engage tissue, such as valve tissue (e.g., native leaflet) or tissue surrounding the valve being replaced, and to cut the tissue (e.g., immediately or over time). In various examples, the one or more tissue cutting elements 4103 are activatable (e.g., from a retracted or inactive state to an activated or active state) and are operative to cut or bisect the tissue (e.g., the anterior leaflet of a mitral valve). In various examples, each tissue cutting element 4103 may transition from an inactive position where the tissue cutting element 4103 is inoperable to engage and cut tissue to an active position where the tissue cutting element 4103 is operable to engage tissue and cut tissue.

The tissue cutting element 4103 generally has one or more cutting edges, such as one or more first cutting edges 4103A (e.g., an inner cutting edge) facing in a first direction relative to the prosthetic valve 1000 (e.g., facing radially inward relative to the leaflet frame subcomponent 1200). The tissue cutting element 4103 may additionally, or alternatively, have one or more second cutting edges 4103B (e.g., FIGS. 3D to 3G) facing a second direction relative to the prosthetic valve 1000 (e.g., facing radially outward relative to the leaflet frame subcomponent 1200), where the one or more first cutting edges 4103A and/or the one or more second cutting edges 4103B may be relatively sharp or otherwise be operable for cutting tissue. In some examples, the tissue cutting element 4103 is configured as a double-edged blade with at least one first cutting edge 4103A (e.g., facing radially inward) and at least one second cutting edge 4103B (e.g., facing radially outward). Alternatively, the tissue cutting element 4103 may include a single cutting edge, or be single-edged.

The first cutting edges 4103A and/or the second cutting edges 4103B may extend an entire length of the tissue cutting element 4103, or for less than an entire length of the tissue cutting element 4103. For example, where there is a desire to incorporate both tissue grasping, or controlling features (e.g., similar to the tissue retention features 1218) and cutting features, a portion of the tissue cutting element 4103 may be operable for cutting (e.g., the portion of the tissue cutting element 4103 including a cutting edge) while another portion may be operable to grasp or retain tissue (e.g., a portion of the tissue cutting element 4103 toward the free end 1420 that is free from any cutting edge). As shown in FIG. 1A, the first cutting edge 4103A extends an entire length of the tissue cutting element 4103, from the base 1422 to the free end 1420. As shown in the examples of FIGS. 3C to 3G, the first cutting edges 4103A and/or the second cutting edges 4103B may extend for less than an entire length of the tissue cutting element 4103. Additionally, the prosthetic valve 1000 optionally includes both one or more tissue cutting elements 4103 as well as one or more tissue retention features 1218. In such examples, the one or more tissue cutting elements 4103 may be operable to cut tissue, while the one or more tissue retention features 1218 may be operable to retain or secure tissue, such as the remaining portions of tissue that has been cut with the tissue cutting elements 4103.

In some examples, the tissue cutting element 4103 is operable as an electrosurgical cutting element. The first cutting edge 4103A and/or the second cutting edge 4103B may be configured to cut tissue via electrosurgical techniques. For example, the tissue cutting element 4103 may be configured to direct electrosurgical energy to one or more of the first cutting edges 4103A and/or the second cutting edges 4103B. When the tissue cutting element 4103 is configured as an electrosurgical cutting element, the prosthetic valve 1000 may also include a current source (not shown) electrically coupled with the tissue cutting element 4103 such that the electrical energy can be applied to the tissue cutting element 4103. In some examples, the current source is connected to the tissue cutting element 4103 using one or more wires.

Generally, the tissue cutting element 4103 can be generally located at any position along the longitudinal axis of the leaflet frame subcomponent 1200 provided that the tissue cutting element 4103 is appropriately sized and shaped operable to cut adjacent tissue either before, during or upon nesting of the leaflet frame subcomponent into the anchor frame subcomponent 1100. Notably, in embodiments where the anchor frame subcomponent 1100 is not present (e.g., where the leaflet frame subcomponent 1200 directly engages the native valve orifice), the tissue cutting element 4103 may cut tissue by longitudinally translating the prosthetic valve 1000 (e.g., the leaflet frame subcomponent 1200) and/or activating the tissue cutting element 4103 to an active state.

As indicated in FIGS. 1A, 1B, 2A, and FIGS. 3C to 3G, the tissue cutting element 4103 of the leaflet frame subcomponent 1200 may be situated along the leaflet frame subcomponent 1200 proximate the leaflet frame subcomponent outlet end 1204. In some examples, the base 1422 of the tissue cutting element 4103 forms part of the outlet end of the leaflet frame subcomponent 1200. In other examples, the base 1422 of the tissue cutting element 4103 is situated proximal to the leaflet frame subcomponent outlet end 1204. In other examples, the one or more tissue cutting elements 4103 are located at an intermediate position along the leaflet frame subcomponent 1200, at a position proximate the leaflet frame subcomponent inlet end 1202, or proximal the leaflet frame subcomponent inlet end 1202.

FIGS. 1A, 3D, and 3E are indicative of tissue cutting element configurations that extend as a single, elongate member generally in a single direction. As indicated in FIG. 1A, the tissue cutting element 4103 may be arcuate, or curved to a desired extent. As indicated in FIG. 3D (active state) and FIG. 3E (inactive state), the tissue cutting element 4103 may have various cutting edge configurations. FIG. 3D (active state) and FIG. 3E (inactive state) are illustrative of cutting element configurations including a hooked, angled, or recurved design extending in a first direction, and then back in a second direction at a desired angle.

In various examples, as mentioned above, the one or more tissue cutting elements 4103 project away from the leaflet frame subcomponent 1200 toward the adjacent tissue when the leaflet frame subcomponent 1200 is in the deployed configuration. Each of the one or more tissue cutting elements 4103 may be deployed or transitioned to the active state at a desired stage in a delivery sequence. For example, each or some of the one or more tissue cutting elements 4103 may be transitioned to the active state prior to expansion of the leaflet frame subcomponent 1200, during expansion of the leaflet frame subcomponent 1200 (e.g., when the leaflet frame subcomponent 1200 is partially expanded), following expansion of the leaflet frame subcomponent 1200, prior to expansion of the anchor frame subcomponent 1100, during expansion of the anchor frame subcomponent 1100 (e.g., when the anchor frame subcomponent 1100 is partially expanded), following expansion of the anchor frame subcomponent 1100, prior to nesting the leaflet frame subcomponent 1200 with the anchor frame subcomponent 1100, during nesting of the leaflet frame subcomponent 1200 and the anchor frame subcomponent 1100 (e.g., when the two are partially nested), following nesting of the leaflet frame subcomponent 1200 and the anchor frame subcomponent 1100, or at any delivery stage as appropriate. Notably, in delivery sequences where the prosthetic valve 1000 does not include an anchor frame subcomponent, the foregoing options regarding relative nesting and expansion of the anchor frame subcomponent 1100 are inapplicable.

As indicated by broken lines in FIG. 3C, the tissue retention features 1218 as previously described may be configured as tissue cutting elements 4103 as desired. For example, and as previously referenced, the tissue cutting element 4103 shown in FIG. 3C has additional functionality beyond cutting, similar to those of the tissue retention features 1218. For example, one of the tissue retention features 1218 may be modified to include sharpened points, edges or other features along a portion thereof to define the tissue cutting element 4103. As shown, the tissue cutting element 4103 in FIG. 3C has a looped configuration, in a similar manner to the tissue retention features 1218, having an apex and two ends, wherein the two ends are coupled to, integral with, extend from, or otherwise terminate into one or more portions of the leaflet frame subcomponent 1200. In further examples, the tissue cutting element 4103 is alternatively looped substantially radially, extending radially outward from the leaflet frame subcomponent 1200 (e.g., in a tear-drop or other shape).

As shown in broken lines, the tissue cutting element 4103 includes a plurality of first cutting edges 4103A that extend radially inward toward the leaflet frame subcomponent 1200. In different terms, the bases 1222 of one or more of tissue retention features 1218 may have one or more cutting edges to form one or more tissue cutting elements 4103. For example, the first side 1228 of one or both of the first leg 1224 and the second leg 1226 of the tissue retention features 1218 may be relatively sharp such that one or both define first cutting edges 4103A toward the base 1222. Thus, as indicated, the free end 1420 of the tissue cutting element 4103 may correspond to the free end 1220 of the tissue retention feature 1218 and the base 1422 of the tissue cutting element 4103 may correspond to the base 1222 of the tissue retention feature 1218.

In some examples, where the tissue cutting element 4103 includes both retention and cutting features, the tissue cutting element 4103 engages with the tissue to help prevent the tissue from interfering with or otherwise obstructing the flow of fluid (e.g., blood) downstream to the prosthetic valve 1000 after the prosthetic valve 1000 has been deployed. The tissue cutting element 4103 assists both with controlling the valve tissue (e.g., hooking or gasping the tissue) while also cutting the tissue in a controlled manner. For example, in mitral valve repair/augmentation procedures, the bisection or other cutting of the anterior leaflet of the mitral valve and control of the remaining one or more cut portions may help minimize the potential for the anterior leaflet to extend or deflect into the left ventricle and create a left ventricle outflow tract obstruction.

In some examples, the tissue cutting element 4103 has a sufficiently sharp edge to cut through the adjacent tissue. In some examples, the tissue cutting element 4103 is relatively dull so as to prevent the tissue from being cut immediately, but rather the tissue cutting element 4103 can be placed against the tissue such that the tissue cutting element 4103 can cut or erode through the tissue over time. Stated in different terms, the tissue cutting element 4103 may be configured to apply friction to slowly cut (e.g., tear, erode, wear) through the tissue to allow the tissue cutting element 4103 to pass through the tissue over a prolonged period of time. The prolonged period of time as described may be predetermined, such as between one (1) week and one (1) month after the prosthetic valve 1000 is implanted. In some examples, the tissue cutting element 4103 may have a shape that assists in cutting the tissue, such as a serrated edge. In some examples, the tissue cutting element 4103 may be activated after a desired time has passed to cause the tissue to be cut. For example, the tissue cutting element 4103 may be an electrosurgical blade which, when electrically activated after the prosthetic valve 1000 is implanted, cuts through the tissue in a short time, such as within seconds. In some examples, whether sharp or dull, the tissue cutting element 4103 may be formed using a bioresorbable material such that the tissue cutting element 4103 may gradually be dissolved into the body of the patient when no longer needed.

In some examples, the definition of a “dull” cutting element may be defined as a cutting element with an edge having a radius of curvature greater than 10 μm, for example from 10 μm to 20 μm, 20 μm to 30 μm, 30 μm to 40 μm, 40 μm to 50 μm, 50 μm to 60 μm, 60 μm to 70 μm, 70 μm to 80 μm, 80 μm to 90 μm, 90 μm to 100 μm, greater than 100 μm, or any value or range included in the foregoing values or any approximate value or range included in the foregoing values. In some examples, the definition of a “sharp” cutting element may be defined as a cutting element with an edge having a radius of curvature less than or equal to 10 μm, for example from 0.1 μm to 1 μm, 1 μm to 2 μm, 2 μm to 3 μm, 3 μm to 4 μm, 4 μm to 5 μm, 5 μm to 6 μm, 6 μm to 7 μm, 7 μm to 8 μm, 8 μm to 9 μm, 9 μm to 10 μm, or any value or range included in the foregoing values or any approximate value or range included in the foregoing values.

In some examples, the prosthetic valve 1000 is configured for implantation to replace a damaged or faulty mitral valve and the tissue cutting element 4103 of the leaflet frame subcomponent 1200 is positionable to be in contact with, and to control or cut the mitral valve anterior leaflet, that is the leaflet of the mitral valve that is closest to the left ventricular outflow tract (“LVOT”).

Regardless, in some examples, the tissue cutting element 4103 projects away from the leaflet frame subcomponent 1200 such that the free end 1420 of the tissue cutting element 4103 is more radially offset from an axis of the leaflet frame subcomponent 1200 (e.g., extends more radially outwardly) than is the base 1422 of the tissue cutting element 4103. In other words, in various examples, the tissue cutting element 4103 is angled relative to a longitudinal axis of the leaflet frame subcomponent 1200 and/or the leaflet frame subcomponent exterior surface 1208 of the leaflet frame subcomponent 1200 when the leaflet frame subcomponent 1200 is in the deployed configuration.

In various examples, a length and angle configuration of the one or more tissue cutting elements 4103 is based on the relative sizes of the anchor frame subcomponent 1100 and the leaflet frame subcomponent 1200. For example, the length and angle configuration of the tissue cutting element 4103 is such that the tissue cutting element 4103 does not prevent or otherwise obstruct the leaflet frame subcomponent 1200 from telescoping or otherwise being nested with the anchor frame subcomponent 1100. Additionally, however, the length and angle configuration of the tissue cutting element 4103 is one that provides for the tissue cutting element 4103 engaging the native leaflet closest to the LVOT of the patient's anatomy, as discussed herein.

In some examples, the tissue cutting element 4103 defines a length of projection L, an angle of project A, and a radius of projection R. As indicated on FIGS. 3D-3G, the length of projection L is overall “height” or profile of the tissue cutting element 4103 in a longitudinal direction. In turn, the angle of projection A is the angle at which the tissue cutting element 4103 extends relative to the longitudinal axis of the prosthetic valve 1000 (or other types of implant). Finally, the radius of projection R is the total distance the tissue cutting element 4103 extends transversely relative to the longitudinal axis.

In some examples, the tissue cutting element 4103 has a length of projection L from one (1) to twenty (20) millimeters in the active configuration, although a variety of values are contemplated. In some examples, the tissue cutting element 4103 has a radius of projection R from one (1) to twenty (20) millimeters in an active configuration, although a variety of values are contemplated. In some examples, the tissue cutting element 4103 has an angle of projection A between 0 and 90 degrees in the active configuration, though as the angle of project A approaches zero (0) degrees and ninety (90) degrees, the ability of the tissue cutting element 4103 to engage and cut tissue may diminish depending upon the particular configuration employed.

In some examples, the tissue cutting element 4103 projects away from the longitudinal axis of the prosthetic valve 1000 (e.g., the longitudinal axis of the leaflet frame subcomponent 1200) with the projection angle A being in a range from thirty (30) to sixty (60) degrees in the active configuration, although a variety of lengths and angular projections are contemplated, including approximate values of any of the foregoing. In some examples, the tissue cutting element 4103 has a length of projection between five (5) and ten (10) millimeters with the angle of projection A in a range from forty (40) to fifty (50) degrees from the longitudinal axis of the prosthetic valve 1000 in the active configuration, although a variety of lengths and angular projections are contemplated, including approximate values of any of the foregoing. In some examples, the tissue cutting element 4103 defines an angle of projection A between fifteen (15) and forty-five (45) degrees relative to a longitudinal axis of the prosthetic valve 1000 (e.g., the longitudinal axis of the leaflet frame subcomponent 1200) in the active configuration, although a variety of lengths and angular projections are contemplated, including approximate values of any of the foregoing. For instance, in some examples, when deployed, the tissue cutting element 4103 of the leaflet frame subcomponent 1200 defines an angle of projection A of approximately thirty (30) degrees relative to a longitudinal axis of the prosthetic valve 1000. Generally, the tissue cutting element 4103 may be angled less than fifteen (15) or alternatively more than forty-five (45) degrees relative to the longitudinal axis, though as the angle approaches zero (0) degrees and ninety (90) degrees, the ability of the tissue cutting element 4103 to engage and cut tissue diminishes. Again, although some specific examples of angle and length values have been provided, a variety of lengths and angular projections are contemplated, including approximate values of any of the foregoing examples.

In various examples, the tissue cutting element 4103 is generally oriented such that the free end 1420 of the tissue cutting element 4103 is situated proximal to the base 1422 of the tissue cutting element 4103, or extends away from an inflow side or end of the prosthetic valve 1000. In other examples, the tissue cutting element 4103, or a portion thereof extends toward the inflow side or end of the prosthetic valve 1000. Regardless, the one or more tissue cutting elements are operable to engage tissue at a desired time in the delivery sequence (e.g., as the leaflet frame subcomponent 1200 and the anchor frame subcomponent 1100 are nested in-situ) to cause tissue to be cut during and/or after the prosthetic valve deployment.

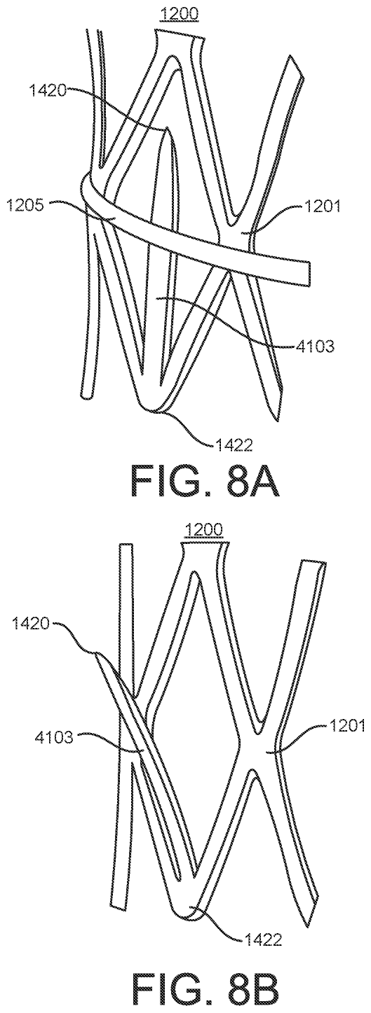

In various examples (e.g., as shown in FIGS. 8A and 8B), while the tissue cutting element 4103 is configured to project away from the portion of the prosthetic valve 1000 to which the tissue cutting element 4103 is coupled (e.g., the leaflet frame subcomponent 1200) when that portion is in the deployed configuration, the tissue cutting element 4103 is stowed or does not otherwise project away from the portion of the prosthetic valve 1000 to which the tissue cutting element 4103 is coupled (e.g., the leaflet frame subcomponent 1200) when that portion is compressed or collapsed in the delivery configuration. In some examples, a constraining member disposed about that portion of the prosthetic valve 1000 (e.g., the leaflet frame subcomponent 1200) during delivery cases stowing of the tissue cutting element 4103 upon deployment of that portion of the prosthetic valve 1000. In some examples, the tissue cutting element 4103 is stowed in associated apertures of the prosthetic valve 1000 (e.g., one or more of the apertures 1216 of the leaflet frame subcomponent 1200). As shown in FIG. 8A, one or more of the frame members 1212 may engage the tissue cutting element 4103 to assist with maintaining the tissue cutting element 4103 in the stowed configuration, or inactive state. Thus, in various examples, the tissue cutting element 4103 is configured to be retained in the inactive state in a stowed or delivery configuration and transitioned to the active state in a projecting or deployed configuration.

From the foregoing, it should be appreciated that the tissue cutting element 4103 may be a resilient structure (e.g., capable of elastic deformation). In some examples, the tissue cutting element 4103 is biased to project away from the leaflet frame subcomponent 1200 (e.g., elastically deformable so as to be self-expanding under spring bias forces) and may be elastically deformed to the inactive state. In other words, in various examples the tissue cutting element 4103 naturally projects away from the leaflet frame subcomponent 1200 and may be transitioned to such a configuration (e.g., upon the leaflet frame subcomponent 1200 expanding to the deployed configuration and/or upon an associated constraining member being removed).

In various examples, the tissue cutting element 4103 is integral to the leaflet frame subcomponent 1200. For example, the tissue cutting element 4103 is formed in conjunction with and from the same material as the frame members 1212. In other examples, the tissue cutting element 4103 is additionally or alternatively coupled to the leaflet frame subcomponent 1200. That is, in some examples, the tissue cutting element 4103 is additionally or alternatively attached to the leaflet frame subcomponent 1200.

FIG. 4 is a side view of the prosthetic valve 1000 in a predeployed configuration, and FIG. 1A is a side view of the prosthetic valve 1000 in a partially deployed configuration (e.g., prior to nesting the anchor frame subcomponent 1100 and the leaflet frame subcomponent 1200) with the interstage 1300 therebetween. FIG. 4 illustrates the prosthetic valve 1000 loaded on a delivery device or delivery device 1500 (e.g., a catheter) in a predeployed configuration with the anchor frame subcomponent 1100 and the leaflet frame subcomponent 1200 being longitudinally offset from one another (also referred to as being delivered in series) and coupled together with the interstage 1300 therebetween. FIG. 1A illustrates the prosthetic valve 1000 in a partially deployed configuration prior to nesting the anchor frame subcomponent 1100 and the leaflet frame subcomponent 1200 with the interstage 1300 everted therebetween. As shown, in both the predeployed and partially deployed configurations, the anchor frame subcomponent 1100 and the leaflet frame subcomponent 1200 are longitudinally offset relative to one another. In some examples, prior to nesting the anchor frame subcomponent 1100 and the leaflet frame subcomponent 1200, the leaflet frame subcomponent inlet end 1202 of the leaflet frame subcomponent 1200 is positioned distal to the anchor frame subcomponent outlet end 1104 of the anchor frame subcomponent 1100 with the interstage 1300 coupled thereto and positioned therebetween coupling them together.

With continued reference to the non-limiting illustrated example of FIG. 4, in the predeployed configuration, the prosthetic valve 1000 is loaded on a delivery device 1500 such that the anchor frame subcomponent 1100 and the leaflet frame subcomponent 1200 are longitudinally offset from one another. Specifically, as shown, a leaflet frame subcomponent inlet end 1202 of the leaflet frame subcomponent 1200 is positioned distal to the anchor frame subcomponent outlet end 1104 of the anchor frame subcomponent 1100. Generally, a removable constraining member (not shown), such as a constraining sheath or a constraining tube is disposed about the prosthetic valve 1000 when the prosthetic valve 1000 is in the predeployed configuration, as those of skill in the art should appreciate. The constraining member has been removed in this illustrated example such that the underlying components of the prosthetic valve 1000 that would otherwise be masked or concealed by the constraining member are viewable.

In various examples, the longitudinal separation or offset of the anchor frame subcomponent 1100 and the leaflet frame subcomponent 1200 provides for a low-profile delivery configuration that can be easily tracked through the vasculature of the patient. For instance, by longitudinally offsetting the anchor frame subcomponent 1100 and the leaflet frame subcomponent 1200, a profile of the delivery system can be minimized because, unlike conventional designs, the anchor frame subcomponent 1100, the leaflet frame subcomponent 1200, and the interstage 1300 do not overlap one another during delivery. In some examples, a maximum profile of the delivery device 1500 including the prosthetic valve 1000 and the constraining member (no shown) can be twenty-four French (24F) or less, although a variety of profiles are contemplated.

Additionally, a region 1502 of the delivery device 1500 positioned between the anchor frame subcomponent 1100 and the leaflet frame subcomponent 1200 and adjacent to the interstage 1300 is operable to bend such that the anchor frame subcomponent 1100 and the leaflet frame subcomponent 1200 are temporarily misaligned with one another. In some examples, such a configuration is akin to rail cars navigating a curve. Such a configuration is beneficial in procedures where the prosthetic valve 1000 is delivered to a treatment region transseptally, which may require a delivery device to bend ninety (90) degrees or more within the left atrium of the heart.

In various examples, upon removing a constraining member (not shown) in-situ, the prosthetic valve 1000 is operable to adopt a partially deployed configuration. In some examples, when in the partially deployed configuration, despite having expanded relative to the predeployed delivery profile, the anchor frame subcomponent 1100 and the leaflet frame subcomponent 1200 remain longitudinally offset relative to one another. For example, as shown in FIG. 1A, the anchor frame subcomponent 1100 and the leaflet frame subcomponent 1200 are longitudinally offset from one another such that the leaflet frame subcomponent inlet end 1202 of the leaflet frame subcomponent 1200 is positioned distal to the anchor frame subcomponent outlet end 1104 of the anchor frame subcomponent 1100 with the interstage 1300 therebetween.

In various examples, after deploying the prosthetic valve 1000 to the predeployed configuration, the anchor frame subcomponent 1100 and the leaflet frame subcomponent 1200 can be nested with one another, with the interstage 1300 being everted therebetween, in-situ. That is, in various examples, the prosthetic valve 1000 can be percutaneously delivered to a treatment region of a patient's anatomy with the anchor frame subcomponent 1100 and the leaflet frame subcomponent 1200 longitudinally offset relative to one another (e.g., an inlet end of the leaflet frame subcomponent 1200 being positioned distal to an outlet end of the anchor frame subcomponent 1100), and subsequently nested with one another (e.g., an inlet end of the leaflet frame subcomponent 1200 being repositioned to a position proximal to an outlet end of the anchor frame subcomponent 1100) in-situ.

### Deployment and Nesting Sequence

FIGS. 5A through 5F illustrate a non-limiting exemplary deployment sequence and nesting configuration of the prosthetic valve 1000 in-situ during a mitral valve (“MV”) replacement procedure, with a cross-section of a portion of the heart for illustrative purposes. In FIG. 5A, the left atrium (“LA”) is accessed transseptally by a delivery device 1500. In various examples, the delivery device 1500 delivered percutaneously and is coupled to a control system 1600 outside of the body. Accessing the left atrium transseptally can be done in accordance with techniques as known those of skill in the art. Upon gaining access to the left atrium transseptally, the delivery device 1500 is positioned for deployment of the prosthetic valve 1000. For example, as shown in FIG. 5B, the delivery device 1500 is advanced through the mitral valve and into the left ventricle (“LV”). In some examples, advancement of the delivery device 1500 through the mitral valve causes the anterior leaflet (“AL”) positioned opposite to the posterior leaflet (“PL”) of the mitral valve to deflect toward the LVOT in the left ventricle.

In various examples, the delivery device 1500 is positioned such that the prosthetic valve 1000 is properly oriented relative to the mitral valve. As shown in FIG. 5B, the delivery device 1500 is positioned such that the anchor frame subcomponent 1100 is adjacent a mitral valve orifice and the anterior leaflet. In various examples, once properly positioned, a constraining sheath 1504 of the delivery device 1500 is retracted relative to the prosthetic valve 1000, thereby exposing the prosthetic valve 1000. In various examples, the prosthetic valve is disposed about a shaft 1506 of the delivery device 1500.

In various examples, with the prosthetic valve 1000 exposed, the prosthetic valve 1000 expands or is otherwise expanded via the use of one or more expansion aids, including but not limited to one or more inflatable balloons. In some examples, expansion of the prosthetic valve 1000 includes the anchor frame subcomponent 1100 expanding relative to the tissue of the mitral valve. In some examples, such expansion causes the anterior leaflet of the mitral valve to deflect and may obstruct the left ventricular outflow tract (“LVOT”) that leads to the aortic valve (“AV”). In various examples, as the anchor frame subcomponent 1100 expands or is expanded, the one or more tissue engagement features 1118 of the anchor frame subcomponent 1100 engage the tissue surrounding the anchor frame subcomponent 1100 (e.g., the mitral valve orifice) and secure the anchor frame subcomponent 1100 against dislodgement from the surrounding tissue and migration of the anchor frame subcomponent 1100.

In various examples, after the anchor frame subcomponent 1100 is expanded and secured relative to the native valve orifice, the anchor frame subcomponent 1100 and the leaflet frame subcomponent 1200 are nested together. In various examples, nesting of the anchor frame subcomponent 1100 and the leaflet frame subcomponent 1200 in-situ involves proximally advancing the leaflet frame subcomponent 1200 relative to the anchor frame subcomponent 1100. In various examples, the leaflet frame subcomponent is in a collapsed, or intermediate expanded configuration when nested into the anchor frame subcomponent 1100. For example, FIG. 5D illustrates the leaflet frame subcomponent 1200 shown in FIG. 5C from a different side and in a collapsed, or unexpanded configuration (whereas FIG. 5C shows the leaflet frame subcomponent in an expanded configuration engaging the anterior leaflet).

In various examples, the leaflet frame subcomponent 1200 is proximally advanced relative to the anchor frame subcomponent 1100 by way of proximally withdrawing the delivery device 1500. For instance, in some examples, the delivery device 1500 includes one or more of the constraining members referred to above. In various examples, the constraining members releasably couple the delivery device 1500 to the leaflet frame subcomponent 1200 such that the one or more of the constraining members are operable to transfer a proximal translation of the delivery device 1500 into a proximal translation of the leaflet frame subcomponent 1200.

The constraining members may be configured to maintain functional engagement or coupling between the delivery device 1500 and the leaflet frame subcomponent 1200 after deployment to facilitate in-situ nesting of the anchor frame subcomponent 1100 and the leaflet frame subcomponent 1200. In some such examples, the constraining members include loops of material (e.g., sutures, filaments, tethers or the like) having one or more portions that pass between the leaflet frame subcomponent interior surface 1206 and the leaflet frame subcomponent exterior surface 1208 by extending through the film disposed about the leaflet frame subcomponent 1200, as discussed above. Regardless, in various examples, withdrawing the delivery device 1500 proximally causes the leaflet frame subcomponent 1200 to translate proximally relative to the anchor frame subcomponent 1100 and to be nested within the anchor frame subcomponent 1100 (whether partially nested and only partially overlapping or completely nested and fully overlapping).

In some examples, the delivery device 1500 includes a plurality of independently movable components (e.g., a plurality of catheters) that can be longitudinally advanced and retracted relative to one another. For instance, in some examples, a first moveable component (e.g., a first catheter) can be proximally withdrawn relative to the anchor frame subcomponent 1100 while maintaining a position of a second movable component (e.g., a second catheter) relative to the anchor frame subcomponent 1100. In some such examples, the first moveable component (e.g., the first catheter) may be coupled to the leaflet frame subcomponent 1200 by way of one or more constraining members (as discussed herein) such that proximally withdrawing the first movable component relative to the anchor frame subcomponent 1100 and the second movable component (e.g., the second catheter) causes the leaflet frame subcomponent 1200 to be withdrawn into the anchor frame subcomponent 1100 such that the leaflet frame subcomponent 1200 can be nested with the anchor frame subcomponent 1100.

In some examples, the second moveable component (e.g., the second catheter) may be coupled to the anchor frame subcomponent 1100 by way of one or more constraining members (as discussed herein) that maintaining a position of the second movable component relative to the anchor frame subcomponent 1100 as the first movable component (e.g., the first catheter) is proximally withdrawn relative to the second movable component the second movable component operates to maintain a position of anchor frame subcomponent 1100 such that the leaflet frame subcomponent 1200 can be nested therewith.

In some examples, one or more constraining members (e.g., tethers) extend between the leaflet frame subcomponent 1200 and the delivery device 1500. In some examples, the one or more tethers are coupled to the leaflet frame subcomponent 1200 such that as the delivery device 1500 is withdrawn, the leaflet frame subcomponent 1200 is proximally advanced relative to the anchor frame subcomponent 1100. In some examples, the one or more constraining members are woven through or otherwise disposed about one or more portions of the leaflet frame subcomponent 1200. For instance, in some examples, each of the constraining members forms a loop or similar feature extends about a portion of the leaflet frame subcomponent 1200. In some examples, one or more lock wires releasably secure the one or more tethers to the leaflet frame subcomponent 1200.

In some examples, in addition to proximally withdrawing or advancing the leaflet frame subcomponent 1200, the anchor frame subcomponent 1100 is secured against longitudinal translation during the nesting procedure. In some examples, longitudinal movement of the anchor frame subcomponent 1100 is arrested by the tissue engagement features 1118 of the anchor frame subcomponent 1100 engaging the tissue surrounding the prosthetic valve 1000. In some examples, the delivery device 1500 includes one or more arresting mechanisms that operate to minimize longitudinal movement of the anchor frame subcomponent 1100 during the nesting procedure. In some examples, the delivery device 1500 includes a pushing element that abuts one or more portions of the anchor frame subcomponent 1100 while the leaflet frame subcomponent is proximally advanced.

In various examples, as the leaflet frame subcomponent 1200 is proximally advanced relative to the anchor frame subcomponent 1100, the tissue cutting element 4103 of the leaflet frame subcomponent 1200 is advanced toward the anterior leaflet of the mitral valve and is configured to engage and cut the anterior leaflet of the mitral valve. As discussed above, the tissue cutting element 4103 of the leaflet frame subcomponent 1200 is configured to engage, and in some examples, cut the anterior leaflet of the mitral valve between the anchor frame subcomponent 1100 and the leaflet frame subcomponent 1200 prior to or during the anchor frame subcomponent 1100 and the leaflet frame subcomponent 1200 being configured in a nested configuration.

The leaflet frame subcomponent 1200 may be expanded as shown in FIG. 5C to engage the tissue to be cut (e.g., the anterior leaflet) and then collapsed to be nested within the anchor frame subcomponent 1100 (e.g., as shown in FIG. 5D). In such instances the tissue may be cut as part of that engagement, or “captured” and cut following initial engagement (e.g., during or following component nesting). Or, the leaflet frame subcomponent 1200 may be in the collapsed configuration (e.g., as shown in FIG. 5D) when the tissue to be cut is engaged. In some other examples, the leaflet frame subcomponent 1200 may be additional or alternatively angulated or otherwise translated toward the tissue to be cut (e.g., using a steerable delivery device configuration) to engage the tissue to be cut. Again here, the tissue may be cut as part of that engagement, or “captured” by the tissue cutting element 4103 and cut following initial engagement (e.g., during or following component nesting).

In terms of nesting during deployment, in various examples, the leaflet frame subcomponent 1200 is proximally advanced relative to the anchor frame subcomponent 1100 until the leaflet frame subcomponent 1200 becomes nested within the anchor frame subcomponent 1100. In various examples, unlike the predeployed and partially deployed configurations, in a nested configuration, the leaflet frame subcomponent inlet end 1202 of the leaflet frame subcomponent 1200 is positioned proximal to the anchor frame subcomponent outlet end 1104 of the anchor frame subcomponent 1100.

FIG. 5E illustrates the leaflet frame subcomponent 1200 nested within the anchor frame subcomponent 1100 such the leaflet frame subcomponent inlet end 1202 of the leaflet frame subcomponent 1200 is positioned proximal to the anchor frame subcomponent outlet end 1104 of the anchor frame subcomponent 1100.

FIG. 5F illustrates the leaflet frame subcomponent 1200 as shown in FIG. 5E from a different side, showing the anterior leaflet after being cut by the tissue cutting element 4103 (or in other examples, not immediately cut but translated or positioned by a tissue cutting element) to form two smaller native leaflet portions (both labeled “AL” in the figure) with a gap (labeled “G” in the figure) therebetween.

In various examples, with one or more of the anterior leaflet of the mitral valve engaged and/or cut by the tissue cutting element 4103 of the leaflet frame subcomponent 1200, the native leaflet is proximally advanced away from the left ventricle (and the left ventricle outflow tract in particular) and toward the left atrium as the leaflet frame subcomponent 1200 is proximally advanced relative to the anchor frame subcomponent 1100. In various examples, this action of cutting at least a portion of the anterior leaflet of the mitral valve helps prevent the anterior leaflet from obstructing or otherwise interfering with the left ventricular outflow tract. For example, as illustrated in FIGS. 5C and 5D, when the prosthetic valve 1000 is deployed, the anterior leaflet of the mitral valve is deflected toward the left ventricular outflow tract (LVOT).

In various examples, if not cut or translated or positioned by a tissue cutting element as illustrated and described herein, the deflected anterior leaflet of the mitral valve extends into the left ventricle and causes a narrowing of, a restriction of, and/or an obstruction of the left ventricular outflow tract. This narrowing, restriction, and/or obstruction of the left ventricular outflow tract can lead to a number of health risks and complications as those of skill in the art will appreciate. By providing a prosthetic valve and method of implanting the same that operates to cut at least a portion of the anterior leaflet of the mitral valve to prevent the anterior leaflet from obstructing or otherwise interfering with the left ventricular outflow tract, the prosthetic valve 1000 of the present application operates to minimize or eliminate the risks associated with a narrowing, restriction, and/or obstruction of the left ventricular outflow tract.

FIG. 5E is an illustration of the prosthetic valve 1000 in a fully deployed configuration wherein the anchor frame subcomponent 1100 and the leaflet frame subcomponent 1200 are nested and the anterior leaflet of the mitral valve is at least partially cut by the tissue cutting element 4103. In some examples, the prosthetic valve 1000 is fully deployed and operational upon the interlock features 1120 coupling together the anchor frame subcomponent 1100 and the leaflet frame subcomponent 1200. As discussed above, the interlock features 1120 are operable to adopt an engaged configuration wherein they engage the leaflet frame subcomponent 1200 and minimize relative axial translation between the anchor frame subcomponent 1100 and the leaflet frame subcomponent 1200 upon the leaflet frame subcomponent 1200 being proximally advanced a designated amount relative to the anchor frame subcomponent 1100.

The posterior and anterior leaflets of the valve are coupled to papillary muscles (“PM”) within the left ventricle via the chordae tendineae (“CT”). Generally, the chordae tendineae are inelastic tendons attached at one end to papillary muscles in the left ventricle, and at the other to the valve cusps of the posterior and anterior leaflets. As mentioned above, the tissue cutting element 4103 generally include free end 1420 that extends from base 1422 and the leaflet frame subcomponent 1200. The free end 1420 may be configured to penetrate between the chordae tendineae to engage with the anterior leaflet. In some instances, the free end 1420 may be distal of the base 1422 and the free end 1420 may have a shape that can engage tissue, for example, the free end 1420 may have a bend or hook (not shown) and a portion that extends proximal.

FIGS. 6A and 6B are cross-sectional views of a prosthetic valve 1000 according some embodiments after the prosthetic valve 1000 has been deployed. As shown, the tissue cutting element 4103 is coupled to and extends from the anchor frame subcomponent 1100 instead of from the leaflet frame subcomponent 1200. FIG. 6A shows an example in which the tissue cutting element 4103 is longitudinally translatable from a first position (shown in broken lines) to a second position (shown in solid lines) to engage and cut tissue, such as the anterior leaflet (AL). As shown in FIG. 6A, the anchor frame subcomponent 1100 and the leaflet frame subcomponent 1200 are nested together such that the leaflet frame subcomponent 1200 is coaxially received within the anchor frame subcomponent interior region 1110 (FIG. 3B) of the anchor frame subcomponent 1100. The extended position may be achieved via a slide, or track arrangement permitting the cutting element to retract longitudinally after initial positioning. Sutures, or similar tensioning elements, may be secured to a portion of the tissue cutting element 4103 to pull, or retract the tissue cutting element 4103. During such longitudinal sliding, the anterior leaflet (AL) may be cut. As shown, the tissue cutting element 4103 is positioned against a surface of the anterior leaflet (AL) of the mitral valve. As in examples previously referenced, the tissue cutting element 4103 may be configured to be sufficiently dull such that, as time passes and with the tissue cutting element 4103 positioned against the anterior leaflet of the mitral valve and exerting a predetermined amount of force against the anterior leaflet, the tissue cutting element 4103 slowly cuts into the surface of the anterior leaflet and thereafter cuts through the anterior leaflet. For example, the tissue cutting element 4103 may be biased in the longitudinal direction toward its final position, but with insufficient force to immediately cut the anterior leaflet (AL). In such an instance, the tissue cutting element 4103 will move longitudinally over time as the anterior leaflet (AL) is cut over time.

As shown in FIG. 6B, the tissue cutting element 4103 may also be biased to swing, or arc from a first position (shown in broken lines) to a second position (shown in solid lines) to achieve a cutting operation. The tissue cutting element 4103 may be elastically biased (e.g., via shape memory) to return to the second position. The cutting operation may occur immediately, with the bias force being sufficient to cut tissue when the tissue cutting element 4103 is released. The cutting operation may also occur over time, via erosion, with the tissue cutting element 4103 slowly returning to the second position. Thus, the tissue cutting element 4103 may be configured to cut tissue over time (e.g., via an erosion type of interaction) or the tissue cutting element 4103 may be configured to more quickly (e.g., immediately, or nearly immediately) cut tissue.

In various examples, the interstage 1300 extends between the anchor frame subcomponent 1100 and the leaflet frame subcomponent 1200 in the nested configuration (e.g., as shown in FIGS. 6A and 6B). In various examples, in addition to coupling the anchor frame subcomponent 1100 with the leaflet frame subcomponent 1200, the interstage 1300 operates to obstruct undesirable retrograde flow through the prosthetic valve 1000. In particular, the film extending between the anchor frame subcomponent 1100 and the leaflet frame subcomponent 1200 in the nested configuration operates to prevent retrograde flow through the annular region defined between the anchor frame subcomponent 1100 and the leaflet frame subcomponent 1200. Thus, while the leaflets of the prosthetic valve 1000 are configured to close and prevent retrograde flow through the prosthetic valve 1000 (and an interior region of the leaflet frame subcomponent in particular), the interstage 1300 extending between the anchor frame subcomponent 1100 and the leaflet frame subcomponent 1200 also operates to minimize or prevent unintended retrograde flow through the prosthetic valve 1000.

Additionally, as shown in FIGS. 6A and 6B, the interlock features 1120 of the anchor frame subcomponent 1100 engage the leaflet frame subcomponent 1200 and operate to maintain a relative position of the leaflet frame subcomponent 1200 with the anchor frame subcomponent 1100. In various examples, the interlock features 1120 of the anchor frame subcomponent 1100 operated to minimize the potential for the leaflet frame subcomponent 1200 to dislodge distally from its nested position within the anchor frame subcomponent 1100. In various examples, the interlock features 1120 extend from the anchor frame subcomponent 1100 to a position distal to one or more of the leaflet frame subcomponent outlet end 1204 of the leaflet frame subcomponent 1200 and the leaflet frame subcomponent inlet end 1202 of the leaflet frame subcomponent 1200. That is, in some examples, the interlock features 1120 extend to and engage a portion of the leaflet frame subcomponent 1200 between the leaflet frame subcomponent inlet and outlet ends 1202 and 1204 thereof. In other examples, in the nested configuration, the interlock features 1120 extend to a position distal to the leaflet frame subcomponent outlet end 1204 of the leaflet frame subcomponent 1200.

Additionally, as shown in FIGS. 6A and 6B, the tissue engagement features 1118 of the anchor frame subcomponent 1100 extend away from the anchor frame subcomponent 1100 and engage the tissue of the valve orifice surrounding the prosthetic valve 1000. In some examples, the tissue engagement features 1118 are configured to penetrate the tissue or otherwise embed within the tissue. In various examples, this interaction of the tissue engagement features 1118 of the anchor frame subcomponent 1100 with the tissue surrounding the prosthetic valve 1000 operates to secure the anchor frame subcomponent 1100 (and thus the leaflet frame subcomponent 1200) to the tissue (e.g., the valve orifice).

The anchor frame subcomponent inlet end 1102 of the anchor frame subcomponent 1100 illustrated in FIGS. 6A and 6B is flared radially outward and is situated adjacent to and in abutment with the valve orifice, as shown. In some examples, such a configuration provides that the anchor frame subcomponent inlet end 1102 of the anchor frame subcomponent 1100 obstructs or otherwise limits the extent to which the anchor frame subcomponent 1100 is operable to extend through the valve. For instance, in the case of a mitral valve replacement, such a flared end at the anchor frame subcomponent inlet end 1102 limits the extent to which the anchor frame subcomponent 1100 can be advanced through the natural mitral valve orifice and into the left ventricle. In some examples, a flared configuration for the anchor frame subcomponent inlet end 1102 additionally operates to minimize the potential for the anchor frame subcomponent 1100 to migrate distally.

While the embodiments and examples illustrated and described above pertain to trans-septal delivery, it should be appreciated that a variety of additional well-known delivery procedures can be utilized without departing from the spirit or scope of the present application. Additional non-limiting delivery procedures include trans-apical, left atriotomy, and trans-aortic. Generally, regardless of the particular delivery procedure, those of skill should appreciate that after deploying the prosthetic valve 1000, the leaflet frame subcomponent 1200 and the anchor frame subcomponent 1100 are nested by proximally advancing the leaflet frame subcomponent 1200 relative to the anchor frame subcomponent 1100.

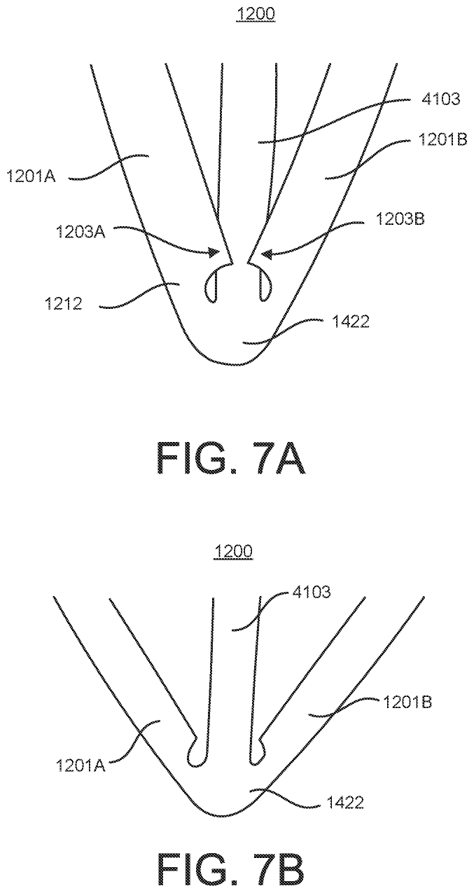

FIGS. 7A and 7B show a method of activating the tissue cutting element 4103 to transition from the inactive position (FIG. 7A) to the active position (FIG. 7B) according to an embodiment. In FIG. 7A, the tissue cutting element 4103 is maintained in the inactive position by two neighboring frame elements or frame members 1201A and 1201B of the leaflet frame 1201. Specifically, in this position, each of the frame members 1201A and 1201B has overlapping portions 1203A or 1203B proximate the base 1422 of the tissue cutting element 4103, and these overlapping portions 1203A and 1203B apply a force against the tissue cutting element 4103 to help prevent the tissue cutting element 4103 from transitioning to the active position.

In FIG. 7B, the leaflet frame 1201 is expanded to an intermediate configuration between the compressed configuration and the fully expanded configuration. In the intermediate configuration, the leaflet frame subcomponent 1200 is still compressed to an extent such that the diameter of the leaflet frame subcomponent 1200 is smaller than in the fully expanded configuration, but the leaflet frame subcomponent 1200 is expanded enough such that the distance between the overlapping portions 1203A and 1203B increases to release the tissue cutting element 4103, transitioning it from the inactive position to the active position. Allowing for the leaflet frame subcomponent 1200 to remain in the intermediate configuration may better allow or otherwise facilitate the leaflet frame subcomponent 1200 being drawn into the anchor frame subcomponent 1100.

FIGS. 8A and 8B shows another method of activating the tissue cutting element 4103 to transition from the inactive position (FIG. 8A) where the tissue cutting element 4103 may not engage tissue to the active position (FIG. 8B) where the tissue cutting element 4103 may engage tissue, according to some embodiments. In FIG. 8A, the tissue cutting element 4103 is maintained in the inactive position by a constraining member 1205 (e.g., a tensioned fiber or band of material associated with a delivery system) positioned around the outer periphery of the leaflet frame 1201 so as to impart a force against the tissue cutting element 4103 to maintain the tissue cutting element 4103 in the inactive position. In some examples, the constraining member 1205 is positioned away from the base 1422 of the tissue cutting element 4103 such that less force is required by the constraining member 1205 to maintain the tissue cutting element 4103 in the inactive position. For example, the tissue cutting element 4103 may be biased toward the active state, or position and then released to the active state, or position.

In some examples, the constraining member 1205 is positioned closer to the free end 1420 of the tissue cutting element 4103 than the base 1422 or at a midpoint between the free end 1420 and the base 1422. The constraining member 1205 may be formed of any of the biocompatible materials described herein, according to the particular function and implementation of the constraining member 1205, whether those described in association with the leaflet frame subcomponent 1200, anchor frame subcomponent 1100, interstage 1300 or other feature of the prosthetic valve 1000. For example, the constraining member 1205 may be formed of a high-tensile strength non-bioresorbable material. Alternatively, the constraining member 1205 may be formed of a bioresorbable or biocorrodible material such that the constraining member 1205 gradually wears away or otherwise degrades over time to eventually release the tissue cutting element 4103.

In some examples, the constraining member 1205 may be tethered or looped through a feature, such as an aperture, (not shown) in the tissue cutting element 4103. This can be advantageous in being able to maintain engagement of the constraining member 1205 and the tissue cutting element 4103 and to control amount of projection of the tissue cutting element 4103.

In some examples, the constraining member includes one or more apertures. For example, the constraining member could be an elongate, elastic member with the ends pinched together such that the constraining member is shorter in one configuration. One or more of the ends, or pinched portions could have a hole or an aperture to receive a tether that extends back toward a user. Upon releasing the tether from the apertures (e.g., by tensioning the tether), the pinched portions are released and the constraining member releases to permit the engaging portion to activate.

It should be readily understood that any of the foregoing methods, including those described in association with FIGS. 7A and 7B as well as of FIGS. 8A and 8B, can be implemented where the tissue cutting element 4103 is coupled to and/or extends from another portion of the prosthetic valve 1000, such as the anchor frame subcomponent 1100 as described above. For reference, FIGS. 9A to 9C illustrate the tissue cutting element 4103 coupled to the anchor frame subcomponent 1100 and extending therefrom.

### Delivery and Deployment Method Including Tissue Cutting

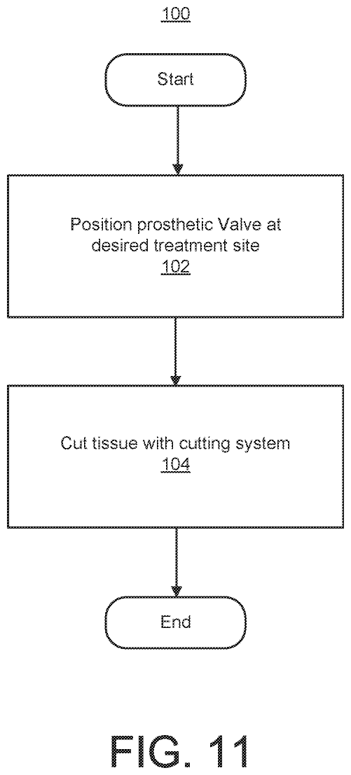

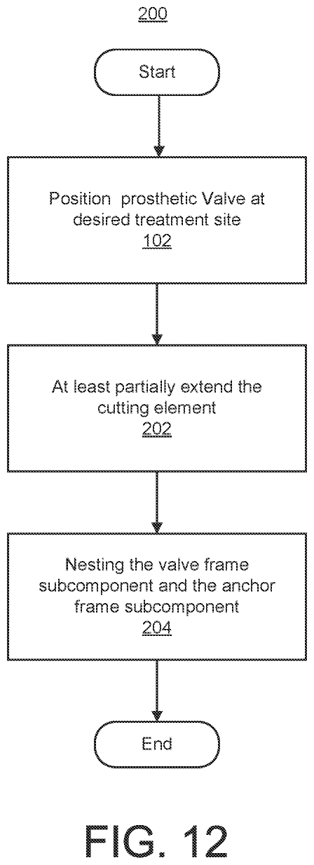

FIGS. 10 and 11 show delivery and deployment methods for prosthetic valves disclosed herein according to some embodiments. In some examples, a method 100 of operation involves positioning a prosthetic valve (for example, the prosthetic valve 1000) at a desired treatment site or location within a body; 102. In some examples, the treatment site is a chamber inside the heart. Target tissue, such as a native leaflet is engaged, so as to cut (e.g., whether immediately or over a desired time period), with one or more tissue cutting elements 4103 included in the prosthetic valve 1000 so as to form two or more smaller native leaflet portions to reduce or prevent obstruction of the LVOT; 104. In some examples, the one or more tissue cutting elements 4103 are configured to additionally retain or grasp the native leaflet (e.g., prior to, during, and/or after the two or more portions are formed).

Some delivery and deployment methods 200 involve the aforementioned step 102 with the prosthetic valve in an unnested, or extended state followed by at least partially extending the one or more tissue cutting elements 4103 to the active state such that the one or more tissue cutting elements 4103 project radially outward relative to at least one of the anchor frame subcomponent 1100 and/or the leaflet frame subcomponent 1200; 202. In some examples, the one or more tissue cutting elements 4103 are positioned past a free edge of the tissue that is to be cut (e.g., beyond the free edge of the anterior leaflet). In some examples, the one or more tissue cutting elements 4103 are configured to additionally retain or grasp the native leaflet (e.g., prior to, curing, and/or after the two or more portions are formed).

Thereafter, the tissue is cut before, during, or after the leaflet frame subcomponent 1200 of the prosthetic valve 1000 is telescopically received, or nested into the anchor frame subcomponent 1100; 204. For example, during the nesting process, the tissue cutting element 4103 may cut the tissue as the tissue cutting element 4103 is moved longitudinally.

In some examples, cutting the tissue is achieved by disposing the one or more tissue cutting elements 4103 of the prosthetic valve 1000 against tissue for a prolonged period of time for the tissue cutting element to gradually cut the tissue (e.g., where the one or more tissue cutting elements 4103 is relatively dull so as to prevent the tissue from being cut immediately, as previously explained). The prolonged period may last between one (1) hour to one (1) month or more after the prosthetic valve is implanted. In some examples, the prolonged period is between one (1) to three (3) weeks, although a variety of periods of time are contemplated. In some examples, the tissue cutting element 4103 may comprise a bioresorbable and/or biocorrodible material such that the tissue cutting element may gradually dissolve in the body of the patient after successfully cutting the tissue.

### Leaflets and Leaflet Constructs

As presented herein, examples of prosthetic valves include flexible leaflets that move in response to changing fluid pressure. The leaflets may be provided as individual components, referred to as leaflets, or may be part of a larger multi-leaflet component referred to as a leaflet construct. It is appreciated that leaflets and leaflet constructs may be constructed in many ways. By way of example, a leaflet may be cut out of a flat sheet or tubular member. By way of another example, a leaflet construct may be cut out of a tubular member or a flat sheet that has been subsequently rolled into a tubular shape. These are but a few examples of forming leaflets or leaflet constructs, which also include, but not limited to, compression and injection molding processes.

For simplicity of discussion, when referring to a material from which a leaflet is made, it is appreciated that the same material may also be used to make a leaflet construct. Therefore, in this context, the term “leaflet” will refer to both leaflet and leaflet construct. It is understood that in embodiments presented herein, the leaflet is flexible and is comprised of a flexible material.

In some examples, each of the one or more leaflets 1210, such as a plurality of the leaflets 1210 forming the leaflet construct 1211, may be formed of a natural material, such as repurposed tissue, including bovine tissue, porcine tissue, or the like.

Examples of suitable biocompatible polymers for use in making a synthetic flexible leaflet include, but are not limited to, the groups including urethanes, silicones (e.g., organopolysiloxanes), copolymers of silicon-urethane, styrene/isobutylene copolymers, polyisobutylene, polyethylene, polyethylene-co-poly(vinyl acetate), polyester copolymers, nylon copolymers, fluorinated hydrocarbon polymers and copolymers and/or mixtures of each of the foregoing, and composite materials made therewith. Examples of a suitable fluoroelastomers include, but are not limited to, copolymers of tetrafluoroethylene and perfluoromethyl vinyl ether (TFE/PMVE copolymer) and (per)fluoroalkylvinylethers (PAVE). Such polymers may exhibit the physical properties of an elastomer, elastomeric, or non-elastomeric material.

Examples of processes to form a synthetic leaflet include, but are not limited to, casting, injection molding, extrusion, and imbibing. In accordance with some embodiments, the leaflet is a composite material that includes at least one membrane as defined above and discussed below combined with a polymer. The polymer may be a coating or layer on the membrane and/or may be imbibed into a microporous structure of the membrane. Examples of a membrane include, but not limited to, microporous polyethylene and expanded fluoropolymer membrane such as expanded polytetrafluoroethylene (ePTFE). The expanded fluoropolymer membrane used to form some of the composite materials described can comprise PTFE homopolymer, blends of PTFE, expandable modified PTFE and/or expanded copolymers of PTFE. As an example of a microporous membrane, ePTFE comprises a matrix of fibrils defining a plurality of spaces within the matrix. The polymer may be imbibed or otherwise incorporated into the plurality of spaces to form the composite material.

It is appreciated that multiple types of membranes and multiple types of polymer can be combined to form a composite material while remaining within the spirit and scope of the present disclosure. Additional materials may be incorporated into the polymer, such as, but not limited to, inorganic fillers, therapeutic agents, radiopaque markers, and the like while remaining within the spirit and scope of the present disclosure. In accordance with some examples, the composite material comprises porous synthetic polymer membrane by weight in a range of about 10% to about 90%.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

By way of example of an elastomer, TFE/PMVE copolymer is an elastomer when comprising essentially of between 60 and 20 weight percent tetrafluoroethylene and respectively between 40 and 80 weight percent perfluoromethyl vinyl ether. By way of example of an elastomeric material TFE/PMVE copolymer is an elastomeric material when comprising essentially of between 67 and 61 weight percent tetrafluoroethylene and respectively between 33 and 39 weight percent perfluoromethyl vinyl ether. By way of example of a non-elastomeric material, TFE/PMVE copolymer is a non-elastomeric material when comprising essentially of between 73 and 68 weight percent tetrafluoroethylene and respectively between 27 and 32 weight percent perfluoromethyl vinyl ether. The TFE and PMVE components of the TFE-PMVE copolymer are presented in wt %. For reference, the wt % of PMVE of 40, 33-39, and 27-32 corresponds to a mol % of 29, 23-28, and 18-22, respectively. The TFE-PMVE copolymer exhibits elastomer, elastomeric, and/or non-elastomeric material properties based on the wt % or mol % of the respective polymers.

In accordance with some embodiments herein, the leaflet comprises a composite material having at least one porous synthetic polymer membrane layer having a plurality of pores and/or spaces and a polymer that is an elastomer and/or an elastomeric material filling the pores and/or spaces of the at least one synthetic polymer membrane layer. In accordance with other examples, the leaflet further comprises a layer or coating of an elastomer and/or an elastomeric material and/or a non-elastomeric material on one or both sides of the composite material. In some examples the leaflet is an expanded polytetrafluoroethylene (ePTFE) membrane having been imbibed with TFE-PMVE copolymer comprising from about 60 to about 20 weight percent tetrafluoroethylene and respectively from about 40 to about 80 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces. In other examples the leaflet is an ePTFE membrane having been imbibed with TFE-PMVE copolymer comprising from about 70 to about 61 weight percent tetrafluoroethylene and respectively from about 33 to about 39 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces.

In addition to expanded fluoropolymer, other biocompatible synthetic polymers may be suitable for use as a porous membrane. As provided below, embodiments comprising microporous polyethylene are provided as a biocompatible polymer suitable for the particular purpose.

### Tissue Ingrowth

In various embodiments, one or more portions of the prosthetic valve 1000 are configured to promote tissue ingrowth. Any portion of the anchor frame subcomponent 1100, leaflet frame subcomponent 1200, interstage 1300, the leaflets 1210 of the leaflet construct 1211, or any other feature or portion of the prosthetic valve 1000 may be constructed in a manner that promotes tissue ingrowth, whether over the entire surface(s) or portion(s) thereof. Moreover, such ingrowth may be selective in depth in that growth may by promoted only into the surface of materials, without growing entirely through the material forming the above noted-features. A variety of membranes, films, coatings, fabrics, or other material configurations may be implemented. Some nonlimiting examples of materials that can be applied to portions of the prosthetic valve 1000 to promote tissue ingrowth include properly configured expanded polytetrafluoroethylene (ePTFE), such as an ePTFE membrane and/or polyethylene terephthalate fabric (e.g., Dacron fabric).

In various embodiments, one or more portions of the leaflet frame subcomponent 1200 may be suitable for promoting tissue ingrowth. For example, the leaflet frame 1201 can be wrapped, covered, or otherwise coupled to a cover material suitable for promoting tissue ingrowth. In various examples, such tissue ingrowth promoting materials can be applied to leaflet frame entirely, or to less than all of the leaflet frame as desired. Similarly, one or more portions of the anchor frame subcomponent 1100 may be suitable for promoting tissue ingrowth. For example, the anchor frame 1101 can be wrapped, covered, or otherwise coupled to a cover material suitable for promoting tissue ingrowth. In various examples, such tissue ingrowth promoting materials can be applied to leaflet frame entirely, or to less than all of the leaflet frame 1201 and/or the anchor frame 1101 as desired. For example, suitable materials for promoting tissue ingrowth could be coupled to the inner and/or outer surfaces of the leaflet frame 1201 and/or the anchor frame 1101.

In some embodiments, one or more of the leaflets 1210 may be constructed to encourage tissue ingrowth and proliferation across one or more discrete regions, portions, or sections of one or more of the materials forming the one or more leaflets 1210, or alternatively across an entirety of one or more of the materials forming each of the one or more leaflets 1210. Tissue ingrowth and proliferation may be promoted on an outflow side or surface of one or more of the leaflets 1210, and/or on an inflow side or surface of the leaflets 1210, and/or within one or more materials forming the leaflets.

According to some examples, tissue ingrowth is facilitated by incorporating one or more tissue ingrowth layers into the leaflets 1210 to permit selective growth into one or more surfaces of the one or more leaflets 1210. In some examples, one or more non-ingrowth layers are also incorporated to prevent ingrowth through an entire thickness of the leaflet material, or through unwanted portions of the leaflet. In various examples, material underlying the tissue ingrowth material (e.g., one or more layers) may inhibit tissue ingrowth to limit depth of tissue ingrowth into the leaflet material. Similar concepts may be applied to promote selective tissue ingrowth into the other portions of the prosthetic valve 1000 as referenced above. In other words, any portion of the prosthetic valve 1000 may benefit from incorporation of tissue ingrowth layer(s), as well as one or more non-ingrowth layers as desired.

In some examples, selective promotion of tissue ingrowth into tissue ingrowth layers is facilitated by selectively imbibing, such as with one or more fluoroelastomers, one or more portions of the materials forming the one or more leaflets 1210. Reference to “selectively imbibing” is referring to the act of imbibing a porous material with a filling material at selected portions of the porous material or to a lesser degree leaving a degree of porosity of the porous material. Similar concepts may be applied to promote selective tissue ingrowth into the other portions of the prosthetic valve 1000 as referenced above.

In various embodiments, tissue ingrowth layer materials include expanded fluoropolymer membranes, which may comprise a plurality of spaces within a matrix of fibrils, and are suitable for promoting and supporting the ingrowth of tissue. Other nonlimiting example materials include other biocompatible porous materials such as knit PTFE. However, rather than including a membrane or carrier, in some examples tissue ingrowth layers may be realized in the form of one or more coatings applied to an underlying material.

Though in some examples tissue ingrowth layers include expanded fluoropolymers made from porous ePTFE membranes, it will be appreciated that tissue ingrowth layers may be formed from a number of different types of membranes, including other fluoropolymer membranes, and other biocompatible porous materials such as porous polyethylene membrane and knit PTFE. For instance, suitable expandable fluoropolymers can comprise PTFE homopolymer. In some examples, tissue ingrowth layers can be formed from copolymers of hexafluoropropylene and tetrafluoroethylene, such as fluorinated ethylene propylene (FEP). In some examples, blends of PTFE, expandable modified PTFE and/or expanded copolymers of PTFE can be used. It will thus be appreciated that tissue ingrowth layers may be formed from a variety of different polymeric materials, provided they are biocompatible and possess or are modified to include a suitable microstructure suitable for promoting or supporting tissue ingrowth. In various non-limiting examples, the tissue ingrowth layers may range in thickness from between one micron and four hundred microns, for example, depending on the selected material.

In some examples, polymeric materials for tissue ingrowth layers may include one or more naturally occurring and/or one or more artificially created pores, reliefs, channels, and/or predetermined surface topology, suitable for supporting tissue ingrowth. Other porous materials which can be suitable for use in forming tissue ingrowth layers include but are not limited to groups of urethanes, fluoropolymers, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

While the above-discussed tissue ingrowth layers may generally include membranes, films, knits, or other structures that are bonded, applied, or otherwise attached to underlying layers, in some examples the tissue ingrowth layers may be applied to underlying layers in the form of one or more coatings. In some such examples, coherent irregular networks are distributed or deposited onto one or more portions, regions, sections, areas, or zones of underlying layers. In some examples, such coherent irregular networks are applied to one or more portions of one or more underlying layers to create a surface texture suitable for supporting the ingrowth and proliferation of tissue, as those of skill will appreciate.

In some examples, coherent irregular networks may be selectively applied to one or more discrete or designated sections, portions, or regions of underlying material. In some such examples, the coherent irregular network is applied to the designated areas by masking or otherwise covering those portions of the underlying material where ingrowth of tissue is undesirable.

In various examples, to achieve layers that promotes or otherwise accommodates ingrowth and proliferation of tissue, expanded fluoropolymer membranes may be selectively imbibed, such as with one or more fluoroelastomers, such that the expanded fluoropolymer membrane includes one or more discrete portions, regions, sections, zones, or areas that are free of or are not otherwise imbibed with the elastomeric filler material (or at least are not filled to the extent that the elastomeric filler material operates to prevent tissue ingrowth).

While the above discussed embodiments and examples include applying tissue ingrowth layers to one or more portions of one or more surfaces of an underlying material, or selectively imbibing one or more portions of one or more sides of a membrane of an underlying material with a filler material, it will be appreciated that, in various examples, tissue ingrowth layers may be constructed by both imbibing one or more portions of a membrane forming an underlying material and applying a tissue ingrowth layers to the selectively imbibed underlying material. In various examples, the imbibing material, or filler material, may be varied within tissue ingrowth layers. That is, in some examples, a first portion, area, region, section, or zone of membrane may be imbibed with a first filler material while a second portion, area, region, section, or zone of the membrane may be imbibed with a second filler material.

### Delivery Device

As discussed above, in various examples, the prosthetic valve 1000 is loaded on a delivery device 1500 in a pre-deployed configuration with the anchor frame subcomponent 1100 and the leaflet frame subcomponent 1200 being longitudinally offset from one another (e.g., arranged in series). In various examples, as mentioned above, one or more constraining members releasably and independently couple the leaflet frame subcomponent 1200 and the anchor frame subcomponent 1100 to the delivery device 1500. In various examples, as discussed in greater detail below, the one or more constraining members can be selectively released from the leaflet frame subcomponent 1200 and the anchor frame subcomponent 1100 to facilitate in-situ nesting of the anchor frame subcomponent 1100 and the leaflet frame subcomponent 1200. In some examples, one or more of the constraining members include one or more portions that may be woven through the film(s) disposed about the leaflet frame subcomponent 1200 and the anchor frame subcomponent 1100, such that a longitudinal actuation of the delivery device 1500 is transferrable to one or more of the leaflet frame subcomponent 1200 and the anchor frame subcomponent 1100 via the one or more constraining members.

### Bio-Active Agents

In some embodiment, all or a part of the prosthetic valve, including the leaflets, may be provided with a biologically active (bio-active) agent. Bio-active agents can be coated onto a portion or the entirety of the prosthetic valve, including the leaflet and/or leaflet construct, for controlled release of the agents once the prosthetic valve is implanted.

Such bio-active agents can include, but are not limited to, anti-thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, aminoglutethimide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolatemofetil); angiogenic agents (e.g., vascular endothelial growth factor (VEGF)), fibroblast growth factor (FGF); angiotensin receptor blockers; nitric oxide donors; anti-sense oligonucleotides and combinations thereof; cell cycle inhibitors, mTOR inhibitors, growth factor receptor signal transduction kinase inhibitors; retinoids; cyclin/CDK inhibitors; HMG co-enzyme reductase inhibitors (statins); and protease inhibitors.

Although the embodiments herein may be described in connection with various principles and beliefs, the described embodiments should not be bound by theory. For example, embodiments are described herein in connection with prosthetic valves, more specifically cardiac prosthetic valves. However, embodiments within the scope of this disclosure can be applied toward any valve or mechanism of similar structure and/or function. Furthermore, embodiments within the scope of this disclosure can be applied in non-cardiac applications. The scope of the concepts addressed in this disclosure has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the scop

1. A prosthetic valve comprising:
a support structure;
one or more leaflets coupled to the support structure; and
a tissue cutting element coupled to the support structure.

2. The prosthetic valve of claim 1, wherein the tissue cutting element is integral to the support structure.

3. The prosthetic valve of claim 1, wherein the tissue cutting element is configured to transitioned from an inactive state where the tissue cutting element is inoperable to cut tissue to an active state in which the tissue cutting element is operable to cut tissue.

4. The prosthetic valve of claim 3, wherein the tissue cutting element is configured to be held proximate the support structure in the inactive state when the tissue cutting element is inoperable to cut tissue and to project away from the support structure in the active state when the tissue cutting element is operable to cut tissue.

5. The prosthetic valve of claim 1, wherein the tissue cutting element has one or more cutting edges that are relatively sharp.

6. The prosthetic valve of claim 1, wherein the tissue cutting element has one or more cutting edges that are relatively dull such that the one or more cutting edges are operable to cut through tissue by eroding through the tissue over an extended period of time.

7. The prosthetic valve of claim 1, wherein the tissue cutting element extends a length from a base coupled to a portion of the prosthetic valve to a free end, and further wherein the tissue cutting element includes one or more cutting edges that extend for less than the length of the tissue cutting element.

8. The prosthetic valve of claim 1, wherein the tissue cutting element is configured as an electrosurgical cutting element.

9. The prosthetic valve of claim 1, wherein the tissue cutting element has a first cutting edge facing a first direction and a second cutting edge facing a second direction different than the first direction.

10. The prosthetic valve of claim 1, wherein the support structure includes a leaflet frame subcomponent to which the one or more leaflets and the tissue cutting element are coupled, and, optionally, wherein the support structure further includes an anchor frame subcomponent into which the leaflet frame subcomponent is configured to be at least partially, telescopically received, and, optionally wherein an inner diameter of the anchor frame subcomponent is greater than an outer diameter of the leaflet frame subcomponent.

11. The prosthetic valve of claim 1, wherein the support structure includes an interstage coupling the leaflet frame subcomponent and the anchor frame subcomponent.

12. The prosthetic valve of claim 1, wherein the support structure is configured to retain the tissue cutting element in an inactive state prior to deployment of the support structure.

13. The prosthetic valve of claim 1, wherein the tissue cutting element is configured to grasp and retain tissue in addition to cutting tissue.

14. The prosthetic valve of claim 1, wherein the tissue cutting element is formed of material that is at least one of bioresorbable and biocorrodible.

15. The prosthetic valve of claim 1, wherein the tissue cutting element is elastically deformable so as to be self-expanding under spring bias forces to project away from the support structure.

16. The prosthetic valve of claim 1, wherein the interstage comprises a polymer membrane.

17. The prosthetic valve of claim 1, wherein the prosthetic valve is configured to replace a native mitral valve and the tissue to be cut is that of an anterior leaflet of the native mitral valve.

18. The prosthetic valve of claim 1, wherein the one or more leaflets are flexible leaflets and, optionally, formed of synthetic material.

19. The prosthetic valve of claim 1, wherein the tissue cutting element is a single-edged cutting element.

20. The prosthetic valve of claim 1, wherein the tissue cutting element is a double-edged cutting element.

21. The prosthetic valve of claim 1, wherein the tissue cutting element has a cutting edge with a radius of curvature of between 0.1 μm and 5 μm.

22. The prosthetic valve of claim 1, wherein the tissue cutting element has a cutting edge with a radius of curvature of between 10 μm and 100 μm.

23. A method of delivering the prosthetic valve of claim 1, to a treatment site, the method comprising:
positioning the prosthetic valve at the treatment site; and
engaging the cutting element with native leaflet tissue at the treatment site to cut the native leaflet tissue, and, optionally, to grasp the native leaflet tissue.

24. The method of claim 23, further comprising nesting a portion of the support structure, optionally a leaflet frame subcomponent, that is coupled to the tissue cutting element with another portion of the prosthetic valve, optionally an anchor frame subcomponent.

25. The method of claim 23, wherein the native leaflet tissue is not cut at a time the tissue cutting element is first engaged with the native leaflet tissue.

26. The method of claim 23, wherein the native leaflet tissue is cut at a time the tissue cutting element is first engaged with the native leaflet tissue.
